(19) United States
(12) Patent Application Publication
(10) Pub. No.: US 2025/0283813 A1
(43) Pub. Date: Sep. 11, 2025

(54) ARTICLES AND METHODS FOR ANALYTE CONCENTRATION MEASUREMENTS

(71) Applicant: Sartorius BioAnalytical Instruments, Inc., Bohemia, NY (US)

(72) Inventors: Andrey Klishin, Foster City, CA (US); Yuri Krasov, Castro Valley, CA (US); Vasiliy Popov, Hayward, CA (US); Yuri Osipchuk, Foster City, CA (US); Thomas Kruse, Göttingen (DE); Markus Kampmann, Dortmund (DE); Jens-Christoph Matuszczyk, Goettingen (DE); Jonas Austerjost, Rietberg (DE); Johannes Lemke, Gottingen (DE)

(73) Assignee: Sartorius BioAnalytical Instruments, Inc., Bohemia, NY (US)

(21) Appl. No.: 18/859,788

(22) PCT Filed: Apr. 24, 2023
(86) PCT No.: PCT/US2023/019626

§ 371 Date: Oct. 24, 2024

Related U.S. Application Data
(60) Provisional application No. 63/334,452, filed on Apr. 25, 2022.
(60) Provisional application No. 63/408,279, filed on Sep. 20, 2022.

(51) Int. Cl. G01N 21/55; G01N 30/20; G01N 33/543

(52) U.S. Cl. CPC G01N 21/55; G01N 30/20; G01N 33/543

(57) ABSTRACT

Instruments and associated methods are generally provided. Advantageously, some instruments described herein may be capable of and/or configured to detect a concentration of an analyte in a fluid that is flowing, in a fluid that includes a high concentration of an analyte, and/or in multiple fluids and/or samples of a fluid in rapid succession. Some methods may comprise detecting a concentration of an analyte that is advantageous for one or more of the aforementioned reasons.

### RELATED APPLICATIONS

This application claims priority under 35 U.S.C. § 119(e) to U.S. Provisional Application No. 63/334,452, filed Apr. 25, 2022, and entitled “Articles and Methods for Analyte Concentration Measurements” and U.S. Provisional Application No. 63/408,279, filed Sep. 20, 2022, and entitled “Advanced Control Strategies for Continuous Capture of Monoclonal Antibodies Based on Biolayer Interferometry,” each of which is incorporated herein by reference in its entirety for all purposes.

### FIELD

Instruments for assessing analyte concentrations, and associated methods, are generally described.

### BACKGROUND

Scientific instruments may be used to determine the concentration of analytes in various fluids. However, such instruments may have trouble measuring analytes with high specificity, at high concentrations, with low lag, or with rapid speed.

Accordingly, new instruments and methods are needed.

### SUMMARY

The present disclosure generally describes instruments and methods. The subject matter described herein involves, in some cases, interrelated products, alternative solutions to a particular problem, and/or a plurality of different uses of one or more systems and/or articles.

In some embodiments, a method is provided. The method comprises contacting a probe with a fluid over a first period of time, detecting a variation of a signal over a first period of time, determining the first concentration based on the variation of the signal over the first period of time, and based on the determination of the first concentration, sending instructions to a bioprocessing system. The fluid is flowing over the probe. An analyte is present in the fluid at a first concentration. At least a portion of the analyte becomes immobilized on the probe.

In some embodiments, a method comprises contacting a probe with a fluid over a first period of time. The fluid is flowing over the probe. An analyte is present in the fluid at a first concentration. At least a portion of the analyte becomes immobilized on the probe. The method further comprises detecting a variation of an optical signal over the first period of time. The method further comprises determining the first concentration based on the variation of the optical signal over the first period of time. The optical signal comprises light reflected from an interface internal to the probe and light reflected from the end of the probe.

In some embodiments, a system is provided. The system comprises a first instrument comprising a probe and a detector configured to detect a variation of a signal over a first period of time and a bioprocessing system. The system is configured to supply a fluid from the bioprocessing system to the first instrument. The first instrument is configured to determine a first concentration of an analyte in the fluid while the fluid contacts and flows over the probe based on the variation of the signal over the first period of time. The system is configured to send instructions to the bioprocessing system based on the determination of the first concentration.

In some embodiments, a first instrument is provided. The first instrument comprises a probe and an optical detector configured to detect a variation of an optical signal over a first period of time. The first instrument is configured to determine a first concentration of an analyte in a fluid contacting and flowing over the probe based on the variation of the optical signal over the first period of time. The optical signal comprises light reflected from an interface internal to the probe and light reflected from the end of the probe.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 depicts an instrument comprising a probe and an optical detector, in accordance with some embodiments;

FIG. 2 depicts an instrument in which a probe is optically coupled to an optical detector by an optical cable, in accordance with some embodiments;

FIG. 3 depicts an instrument comprising a probe, an optical detector, and a light source, in accordance with some embodiments;

FIG. 4 depicts an instrument in which a light source is optically coupled to a probe, in accordance with some embodiments;

FIG. 5 depicts an instrument comprising a housing in which a probe is positioned, in accordance with some embodiments;

FIG. 6 depicts an instrument comprising an inlet and an outlet, in accordance with some embodiments;

FIG. 7 depicts an instrument comprising a probe and a microfluidic channel, in accordance with some embodiments;

FIG. 8 depicts an instrument comprising a source of fluid, in accordance with some embodiments;

FIG. 9 depicts of an instrument comprising a valve, in accordance with some embodiments;

FIG. 10 depicts an instrument comprising a plurality of valves, in accordance with some embodiments;

FIGS. 11-12 depict a probe, in accordance with some embodiments;

FIG. 13 depicts an instrument enclosed by an enclosure, in accordance with some embodiments;

FIG. 14 depicts an instrument that comprises two or more parts that are enclosed by separate enclosures, in accordance with some embodiments;

FIG. 15 depicts a process by which the amount of analyte immobilized on a probe can be detected, in accordance with some embodiments;

FIG. 16 depicts a cross-section of a probe comprising an optical fiber, in accordance with some embodiments;

FIGS. 17-18 depict a probe comprising a plastic hub, in accordance with some embodiments;

FIGS. 19-21 show simulated optical signals for equilibrium immobilization of an analyte on a probe, in accordance with some embodiments;

FIGS. 22-24 depict a method in which a probe is contacted with a fluid possibly comprising an analyte and subsequently regenerated and neutralized, in accordance with some embodiments;

FIG. 25 is a graph showing the optical signal (which is indicative of analyte binding), first derivative of the optical signal, and method step in a method comprising regenerating a probe, in accordance with some embodiments;

FIG. 26 depicts an instrument comprising a microfluidic channel, in accordance with some embodiments;

FIGS. 27-30 depict exemplary instruments, in accordance with some embodiments;

FIG. 31 shows the first derivative of the optical signal as a function of time, in accordance with some embodiments;

FIG. 32 shows the measurement times to column breakthrough for a chromatography system, in accordance with some embodiments;

FIG. 33 schematically depicts a biolayer interferometry process, in accordance with some embodiments;

FIG. 34 schematically illustrates a process control strategy, in accordance with some embodiments;

FIG. 35 is a photograph showing a prototype of an instrument, in accordance with some embodiments;

FIG. 36 is a schematic depiction of an instrument, in accordance with some embodiments;

FIG. 37 shows data obtained during a measurement cycle, in accordance with some embodiments;

FIG. 38 shows a control strategy, in accordance with some embodiments;

FIG. 39 shows data obtained during perfusion cell cultivation, in accordance with some embodiments;

FIG. 40 is a schematic depiction of an instrument for performing dynamic flow control and dynamic loading, in accordance with some embodiments; and

FIGS. 41-43 show data obtained during a continuous capture process, in accordance with some embodiments.

### DETAILED DESCRIPTION

Instruments, systems, and associated methods are generally provided. Advantageously, some instruments and systems described herein may be capable of and/or configured to detect a concentration of an analyte in a fluid that is flowing, in a fluid that includes a concentration of an analyte above that which would saturate the immobilization capacity of a probe (e.g., the immobilization capacity of a probe in equilibrium), and/or in multiple fluids and/or samples of a fluid in rapid succession. Some methods may comprise detecting a concentration of an analyte that is advantageous for one or more of the aforementioned reasons. It is also possible for some instruments, systems, and methods described herein to lack one of the aforementioned advantages and/or be performed in conditions other than those mentioned above (e.g., on stationary fluids, on fluids comprising low concentrations of analytes, and/or on a single fluid).

Some methods relate to employing a probe to measure one or more properties of a fluid supplied by a bioprocessing system and then supplying instructions to the bioprocessing system based on such measured property or properties and some systems described herein may be capable of and/or configured to perform such methods. Such methods may be performed for upstream bioprocessing operations and/or downstream bioprocessing operations. Such instructions may include instructions to modify one or more properties of the fluid in the bioprocessing system (e.g., to return them to a particular range), to supply the fluid to a different location (e.g., when the location to which the fluid is supplied can no longer receive any more such fluid or for which it would not be beneficial to supply any more such fluid), to pause (e.g., to allow an operator to troubleshoot, to stop supplying fluid until an operator takes an action), and/or to take no action (e.g., if the measurement indicates that the conditions in the bioprocessing system are acceptable, if it is desirable to continue to supply the fluid to the same location).

Advantageously, methods relating to measuring one or more properties of a fluid supplied by a bioprocessing system and then supplying instructions to the bioprocessing system based on such measured property or properties may allow for one or more features of a bioprocessing system to be monitored during operation thereof and/or for one or more features of a bioprocessing system to maintained in a particular range during bioprocessing system operation. These actions may be performed in an automated manner (e.g., without the need for the attention and/or action of an operator). Allowing bioprocessing system features to be monitored and/or controlled in an automated manner may allow for troubleshooting, logging conditions present during any particular bioprocessing run, and/or quality control. They may also simplify laboratory operations by removing the need for operators to perform functions performed automatically.

Some methods relate to measuring concentrations of analytes in multiple fluid samples using a single probe. Some instruments described herein are capable of and/or configured to perform such measurements. The ability to perform such measurements may beneficially allow for multiple measurements to be performed without the need to change probes, which may enhance the speed at which measurements are performed and/or reduce the expense of performing multiple measurements by reducing the number of probes needed. It may be particularly beneficial to perform such measurements when many measurements need to be performed relatively quickly, such as when the variation over time of the concentration of an analyte in a fluid is being determined and/or when the concentration of an analyte in fluids supplied from various instruments is being determined.

In some embodiments, a method and/or an instrument may make use of a component that facilitates rapid analysis of multiple fluid samples. As one example, in some embodiments, an instrument comprises one or more valves that can be reversibly switched between a plurality of positions. Each valve may be capable of and/or configured to reversibly place one or more sources of fluids in fluidic communication with the probe. Some instruments may comprise a valve that can reversibly switch between placing two or more sources of fluids in fluidic communication with the probe (e.g., in which at least two of the positions between which the valve can be reversibly switched is in fluidic communication with a different source of fluid). Some instruments may comprise one or more valves that directly supply a fluid to the probe. Switchable valves and valves that supply fluids directly to a probe may increase the rate and/or concentration at which a fluid can be provided to a probe in comparison to other methods by which a fluid can be provided to a probe, which may increase the speed at which a signal may be obtained from an interaction between the fluid and the probe. For instance, such valves may supply fluids directly to the probe without further dilution by other sources of fluid supplying different (e.g., diluting) fluids to the probe.

Some methods relate to employing multiple probes. Some instruments described herein are capable of and/or configured to employ multiple probes. Employing multiple probes may advantageously allow for measurements to be made on samples serially obtained from a continuous source of fluid. For instance, measurements may be made on a continuous stream of fluid in which a concentration of an analyte is expected to vary. The continuous stream of fluid may be divided into multiple samples, and the samples may be supplied to the probes in a manner such that a measurement is made on each sample. The presence of multiple probes may allow for one (or more) probe(s) to be employed to contact a sample and immobilize any analyte present therein while one (or more) probe(s) undergo a regeneration process so that it or they can be employed to contact a different sample and immobilize any analyte present therein. Then, the one (or more) probe(s) that have been regenerated can be employed to contact a sample and immobilize any analyte present while the probe(s) previously employed to contact the sample(s) are regenerated. In some such embodiments, any sample being generated can be supplied to a regenerated probe and any probe that has been exposed to a sample can undergo the regeneration process while another probe is being exposed to a different sample, which may allow for measurement of any sample generated upon generation.

Some methods relate to measuring the concentration of an analyte based on the rate at which it becomes immobilized on a probe. Some instruments described herein are configured to and/or are capable of performing such measurements. Measuring the rate at which an analyte becomes immobilized on a probe may advantageously allow for the measurement of higher analyte concentrations with relatively high precision, such as analyte concentrations that would saturate the capacity of the probe to immobilize the analyte, that are close to such concentrations, or that are in excess of such concentrations. Analytes at concentrations that would result in identical or similar amounts of equilibrium immobilization on a probe may become immobilized on the probe at different rates and/or at rates that are easier to distinguish from each other. Accordingly, measuring the rate at which an analyte becomes immobilized on a probe may offer a way to measure analyte concentrations at precisions that may not be possible with other techniques.

Additionally, measuring the rate at which an analyte becomes immobilized on a probe may be a relatively rapid way to measure analyte concentration in a fluid and/or in a sample of a fluid. In some embodiments, a measurement of the rate at which an analyte becomes immobilized on a probe is performed before the analyte reaches its steady-state level of immobilization on the probe and/or on data that is obtained before the analyte reaches its steady-state level of immobilization on the probe. In some embodiments, this period of time is relatively short, which may allow for the concentration of an analyte in a fluid and/or in a sample of a fluid to be determined relatively quickly.

Some methods relate to performing measurements while a fluid and/or a sample of a fluid is flowing across the probe. Some instruments described herein are capable of and/or configured to perform measurements on fluid and/or a sample of a fluid that is flowing across a probe. Making measurements on a fluid and/or a sample of a fluid flowing across a probe may be desirable for applications in which measurements are made on fluid and/or a sample of a fluid being supplied from a source, such as an instrument and/or a component of an instrument. Fluids and/or samples of a fluid being supplied may be flowed across the probe and then into another or the same instrument, another or the same instrument component, or a waste receptacle. This may allow for the probe to be integrated into a flow-through system in which measurements are being made and/or as part of a fluid flow loop. Additionally, it may allow for the probe to be employed to perform multiple measurements and/or undergo multiple processes (e.g., measurement processes, regeneration processes) without being translated and/or with the use of relatively few moving parts.

It should also be understood that the methods described herein may comprise performing measurements while a fluid and/or a sample of a fluid is stationary. As an example, a method may comprise flowing a fluid and/or a sample of a fluid to a location such that it contacts the probe and then performing a measurement while the fluid and/or the sample of the fluid is stationary. Afterwards, the fluid and/or the sample of the fluid may be flowed so that it no longer contacts the probe. It is also possible for one or more of the methods described herein to lack any fluid flow.

As described above, in some embodiments, an instrument is provided. In some embodiments, a system is provided that comprises an instrument. An overview of some exemplary instruments is provided below.

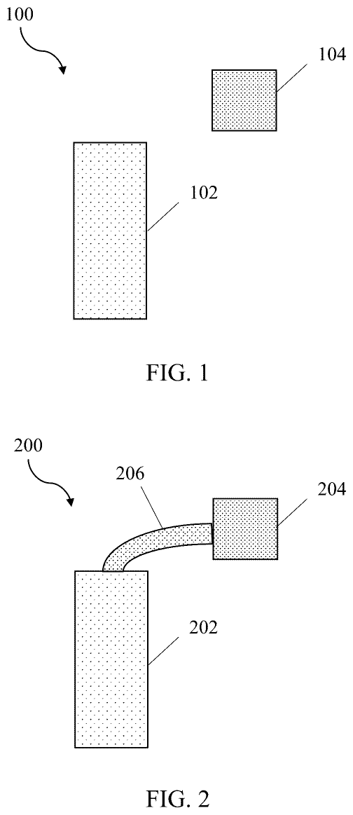

FIG. 1 depicts one example of an instrument. In FIG. 1, the instrument 100 comprises a probe 102 and a detector 104, such as an optical detector. As will be described in further detail below, the probe may have one or more characteristics that allow and/or promote the immobilization of one or more analytes thereon. The detector may be configured to detect a signal. When the detector is an optical detector, it may be configured to detect an optical signal, such as interference. In some embodiments, an instrument comprises a detector that is coupled to a probe, such as an optical detector that is optically coupled to a probe. One such embodiment in shown in FIG. 2, which depicts an instrument 200 in which a probe 202 is optically coupled to an optical detector 204 by an optical cable 206. The optical cable may transmit light from the probe to the optical detector. In some embodiments, an instrument comprises a probe that is optically transparent, and an optical signal to be detected travels through the probe, through an optical cable positioned on a side of the probe opposite the side on which the optical signal is generated, and into an optical detector.

In some embodiments, an instrument comprises multiple probes and/or detectors (e.g., multiple optical detectors) (not shown). In such embodiments, it is possible for each detector (e.g., optical detector) to be associated with (e.g., optically coupled to) a different probe or for two or more probes to be coupled (e.g., optically coupled) to a common detector (e.g., a common optical detector). It is also possible for an instrument to be configured such that one or more probes may reversibly be associated with (e.g., optically coupled to) a detector (e.g., an optical detector) (also not shown). In such embodiments, a detector (e.g., a single optical detector) may be capable of and/or configured to being reversibly associated with (e.g., optically coupled to) two or more probes.

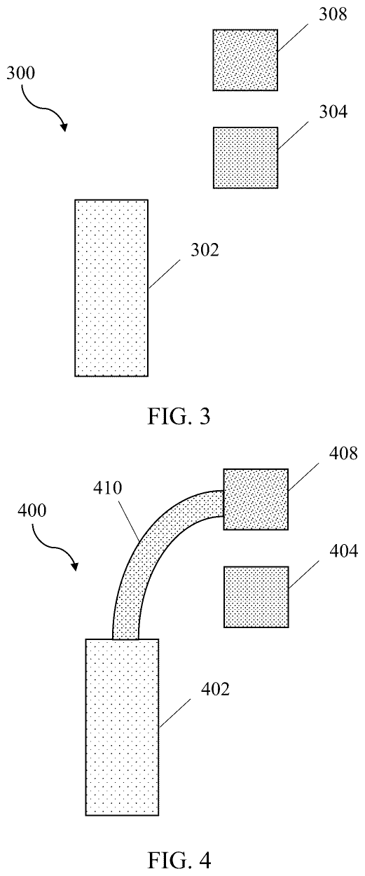

In some embodiments, an instrument comprises one or more components in addition to a probe and a detector (e.g., an optical detector) and/or instead of a probe or a detector (e.g., an optical detector). As an example, in some embodiments, an instrument comprises a light source (e.g., in addition to a probe, in addition to a probe and an optical detector). FIG. 3 shows one example of an instrument comprising a light source. In FIG. 3, the instrument 300 comprises a light source 308. The light source may be capable of and/or configured to illuminate one or more portions of the instrument, such as a probe. In some embodiments, a light source is capable of and/or configured to supply light to a probe that is transmitted through the probe. Light sources may supply light to probes via optical cables. For instance, in some embodiments, an optical cable couples a light source to a probe (e.g., it may transmit light from a light source to a probe). FIG. 4 shows one non-limiting example of such an instrument. In FIG. 4, the light source 408 is optically coupled to the probe 402 by the optical cable 410.

In some embodiments, an instrument comprises a plurality of light sources (not shown). In such embodiments, it is possible for each light source to be associated with (e.g., optically coupled to) a different probe or for two or more probes to be associated with (e.g., optically coupled to) a common light source. It is also possible for an instrument to be configured such that one or more probes may reversibly be optically coupled to a light source (also not shown). In such embodiments, a single light source may be capable of and/or configured to being reversibly optically coupled to two or more probes. This may be accomplished by, for example, an optical switch configured to switch which probe a light source is associated with.

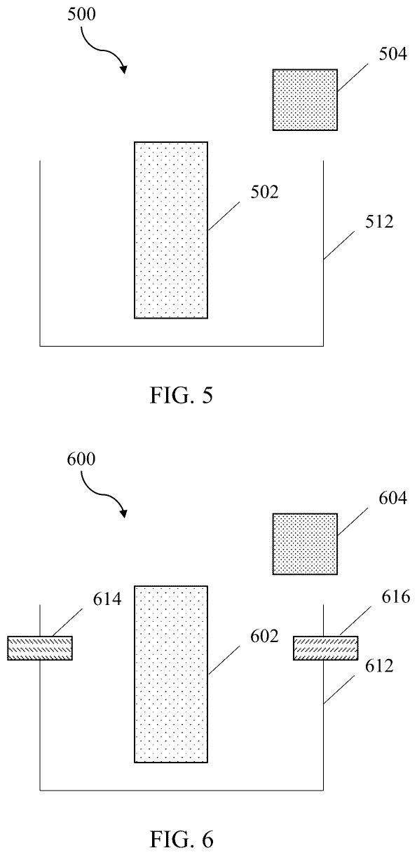

As another example, in some embodiments, an instrument comprises a housing in which the probe and/or the detector (e.g., the optical detector) are positioned. FIG. 5 shows an example of an instrument 500 comprising a housing 512 in which the probe, but not the detector (e.g., the optical detector), is positioned. The housing may be capable of and/or configured to contain one or more fluids and/or one or more samples of a fluid, such as one or more fluids contacted by the probe and/or one or more samples of a fluid contacted by the probe. In some embodiments, a probe contacts, is capable of contacting, and/or is configured to contact a fluid and/or a sample of a fluid positioned in the housing. Fluid may flow into the housing through an inlet and/or out of the housing through an outlet. FIG. 6 depicts an instrument 600 comprising an inlet 614 and an outlet 616. In some embodiments, fluid flows into the inlet (and, in some embodiments, into the housing), across the probe, and then out through the outlet (and, in some embodiments, out of the housing).

Some housings may mechanically support probes and/or position probes in locations at which they may contact fluids and/or samples of a fluid. For instance, in some embodiments, a housing comprises epoxy that provides rigid support the probe and/or protects the probe from damage. In some embodiments, a housing comprises an opening into which a probe can be inserted. Additionally or alternatively, it is also possible for a probe to be mechanically coupled to a housing (e.g., by use of clamps).

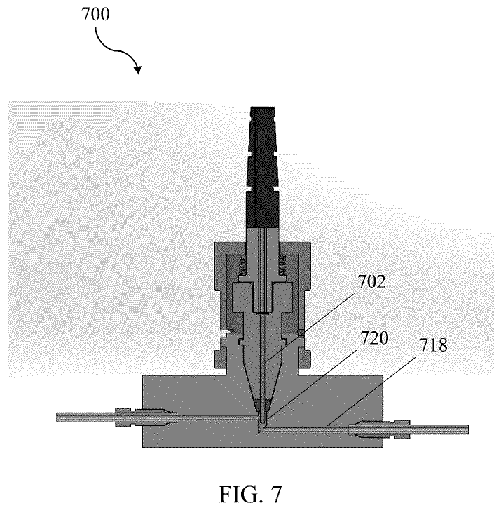

In some embodiments, the housing may form part of a microfluidic chip and/or part of a manifold. When the housing forms part of a manifold, the manifold may supply, be configured to supply, and/or be capable of supplying fluids from sources of fluids to the probe. In some embodiments, a housing forms part of a manifold that comprises one or more microfluidic channels. A probe positioned in such a housing may be in fluidic communication with a microfluidic channel. The microfluidic channels present in the manifolds described herein may have a variety of suitable shapes. In some embodiments, a manifold comprises straight microfluidic channels. It is also possible for a manifold to comprise a microfluidic channel comprising a step and/or a bend. One non-limiting example of such a microfluidic channel is shown in FIG. 7. FIG. 7 depicts an instrument 700 comprising a probe 702 and a microfluidic channel 718. The microfluidic channel shown in FIG. 7 comprises a step 720.

Some microfluidic channels may be perpendicular to the optical axis of a probe in fluidic communication therewith.

A manifold may further comprise one or more components that facilitate fluidic communication between one or more instrument components and the manifold. As two examples, a manifold may further comprise openings and/or tube connectors (e.g., to facilitate fluidic communication between a valve and the manifold, to facilitate fluidic communication between a source of fluid and the manifold). As another example, a manifold may comprise and/or may be employed in conjunction with an O-ring (e.g., to seal a surface between the manifold and a probe). As a third example, a manifold may further comprise luer connections (e.g., to facilitate mechanical connection between a probe and the manifold).

Manifolds may be formed from a variety of suitable materials, such as plastic (e.g., molded plastic, machined plastic) and/or metal (e.g., steel, such as stainless steel). In some embodiments, a manifold comprises two pieces of plastic that are attached together (e.g., via glue, via double-sided adhesive tape). One or more microfluidic channels may be formed in the top piece of plastic. The bottom piece of plastic may assist with insulating the microfluidic channel.

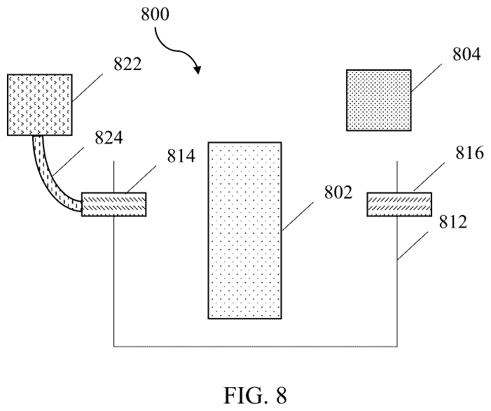

Another example of an additional component that may be included in the instruments described herein is a source of a fluid. Sources of fluids may be associated with, capable of being associated with, and/or associated with the instruments described herein. As an example, a source of fluid may be capable of and/or configured to provide a fluid (and/or one or more samples thereof) to the housing and/or to the probe (e.g., they may be positioned upstream of the housing and/or the probe). As another example, a source of a fluid may be in fluidic communication, capable of being placed in fluidic communication, and/or configured to be placed in fluidic communication with a probe and/or an interior of a housing. The association, provision of a fluid, and/or fluidic communication may occur via an inlet. As an example, and as shown in FIG. 8, a source of a fluid may be configured to be provided to a probe and/or an interior of a housing in which a probe is positioned via an inlet. In FIG. 8, the source 822 of the fluid is in fluidic communication with the inlet 814 via the conduit 824. A variety of suitable conduits may be employed, one non-limiting example of which is tubing. In some embodiments, the tubing may be relatively short. This may facilitate rapid analysis of fluids and/or samples of a fluid supplied to the probe and/or housing and/or reduce the amount of fluid necessary to supply the probe.

In some embodiments, an instrument comprises a plurality of sources of fluid (e.g., more than one source of fluid). For instance, an instrument may comprise two sources of different fluids and/or two sources of the same fluid. In such embodiments, the instrument may comprise a plurality of inlets (e.g., more than one inlet). As an example, an instrument may comprise an inlet associated with each source of fluid and/or with each type of source of fluid. In some embodiments, an instrument comprises exactly one inlet and/or comprises more sources of fluids than inlets. In such embodiments, a source of fluid may be reversibly associated with and/or capable of being reversibly associated with an inlet. Some inlets may be capable of being reversibly associated with a source of fluid and/or with more than one source of fluid.

In some embodiments, an instrument comprises a plurality of inlets and a plurality of probes, and each inlet is in fluidic communication with a probe. Such inlets may also be reversibly in fluidic communication with one or more sources of fluids. Instruments comprising such probes and inlets may be configured to and/or capable of contacting the plurality of probes with one or more fluids supplied via the inlet(s). In some embodiments, an instrument comprises a plurality of probes and a plurality of inlets, and the instrument is configured to contact each probe alternately with different fluids, each of which are alternately supplied by the inlet in fluidic communication with that probe. It is also possible for an instrument to be configured to contact each probe alternately with different fluids, each of which are supplied by a different inlet in fluidic communication with that probe.

Similarly, it is possible for an instrument to comprise one outlet or to comprise more than one outlet. The outlet(s) may be associated with a location to which the probe and/or housing is configured to and/or capable of emitting fluid (e.g., a location downstream from the outlet). As an example, an outlet may place a probe and/or the interior of a housing in fluidic communication with such a location. For instance, in some embodiments, an outlet places a probe and/or interior of a housing in fluidic communication with a waste receptacle (e.g., a waste receptacle downstream from the outlet). As another example, in some embodiments, an outlet places a probe and/or interior of a housing in fluidic communication with a receptacle into which the fluid can be stored (e.g., as a final product, for further processing). As a third example, in some embodiments, an outlet places a probe and/or interior of a housing in fluidic communication with the source of the fluid. In such embodiments, the instrument may serve to recirculate fluid back to a source from which it came after being contacted with the probe. As a fourth example, in some embodiments, an outlet places a probe and/or interior of a housing in fluidic communication with an additional instrument (e.g., that may further process and/or analyze the fluid). It is also possible for an outlet to be reversibly associated with and/or capable of being reversibly associated with one or more locations (e.g., one or more waste receptacles).

When an instrument comprises more than one source of fluid that may be reversibly associated with an inlet and/or more than one location that may reversibly associated with an outlet, the reversible association may be facilitated by the use of a valve. As an example, an instrument may comprise a valve that is configured to switch between a plurality of (e.g., two or more, three or more, four or more, five or more, six or more) positions. Each position (or some of the positions) may be associated with a source of fluid. For instance, each position may place a source of fluid in fluidic communication with a probe with which the valve is in fluidic communication. Switching a valve to a position associated with a source of fluid may place that source of fluid in fluidic communication with the probe. Switching a valve from a position associated with a source of fluid may remove that source of fluid from fluidic communication with the probe. Switching a valve between two positions may remove one source of fluid from fluidic communication with the probe and place a different source of fluid in fluidic communication with the probe.

Valves may be positioned downstream of the source(s) of fluid (e.g., the source(s) of fluid with which the valve is capable of and/or configured to place a probe in fluidic communication may be positioned upstream of the valve), and, possibly, upstream of an inlet. In some embodiments, such a valve supplies a fluid directly to the probe when in an appropriate position to do so. In other words, when the valve places a source of fluid in fluidic communication with the probe, that fluid may flow directly to the probe. While flowing directly the probe from the valve, the fluid may undergo minimal (or no) dilution and/or mixing with other fluids.

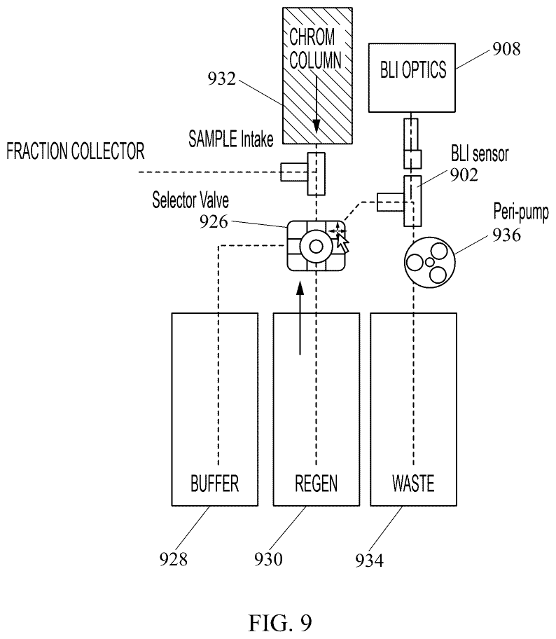

FIG. 9 shows one non-limiting example of an instrument comprising a valve 926 (labeled as “Selector valve” therein). FIG. 9 depicts an instrument comprising three sources of fluid (a source of samples 932 that is labeled “SAMPLE Intake” and “CHROM COLUMN”, a source of a neutralization fluid 928 that is a buffer that is labeled “BUFFER”, and a source of a regeneration fluid 930 that is labeled “REGEN”, each of which will be described in further detail below), a probe 902 (labeled “BLI sensor”), an optical detector 908 (labeled “BLI optics”), and a waste receptacle 934 (labeled “WASTE”). FIG. 9 also depicts a pump 936 that may be employed to facilitate fluid flow from the housing to the waste receptacle (labeled “Peri-pump”).

In some embodiments, an instrument comprises more than one valve. As an example, in some embodiments, an instrument comprises a plurality of valves. Each such valve may be switchable (e.g., as described above) between a plurality of positions, each of which (or some of which) may place the probe in fluidic communication with a source of fluid. It is also possible for some or all such valves to be switchable merely between an open position (i.e., a position that places a probe in fluidic communication with a source of fluid) and a closed position (i.e., a position that does not place a probe in fluidic communication with that source of fluid).

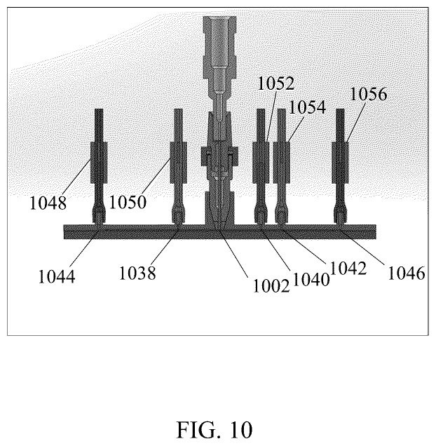

FIG. 10 shows one non-limiting example of an instrument suitable for use with a plurality of such valves. In FIG. 10, the inlets 1038, 1040, and 1042 are each positioned upstream of the probe 1002. Each such inlet may be in fluidic communication with a valve that comprises a position that places a probe in fluidic communication with a source of a different fluid (e.g., the inlet 1038 may be in fluidic communication with a valve that comprises a position that places the probe 1002 in fluidic communication with a source of samples, the inlet 1040 may be in fluidic communication with a valve that comprises a position that places the probe 1002 in fluidic communication with a source of a regeneration fluid, and/or the inlet 1042 may be in fluidic communication with a valve that comprises a position that places the probe 1002 in fluidic communication with a source of a neutralization fluid). FIG. 10 also depicts two outlets that may be in fluidic communication with valves that comprise positions that place the probe 802 in fluidic communication with waste receptacles (the outlets 1044 and 1046). The plurality of valves is shown in FIG. 10 as the valves 1048-1056.

Fluid flowing may flow through the instrument shown in FIG. 10 directly from a valve, across the probe, and then to one of the two waste receptacles. Accordingly, it should be appreciated that fluid may flow through the instrument shown in FIG. 10 in both directions (e.g., from the valves 1040 or 1042 to the waste receptacle 1044, from the valve 1038 to the waste receptacle 1046). Additionally, as can be appreciated from FIG. 10, fluid may flow directly from a valve across the probe without undergoing any dilution or mixing. In some embodiments, such flow may push a plug of another type of fluid (e.g., that supplied from a different valve) downstream and/or to a waste receptacle without substantial (or any) mixing between the two types of fluids.

It should also be noted that, although FIGS. 9 and 10 depict instruments suitable for use with valves that are positioned upstream of inlets, it is also possible for an instrument to comprise a valve that is positioned upstream of a probe but downstream of an inlet. In other words, an instrument may comprise a housing comprising an inlet (or more than one inlet), and one or more valves may be positioned between the inlet (or inlets) and the probe. In some such embodiments, fluids flow into the housing via the inlet but only contact the probe if the valve is in a position that places the inlet from which the fluid enters the housing in fluidic communication with the probe.

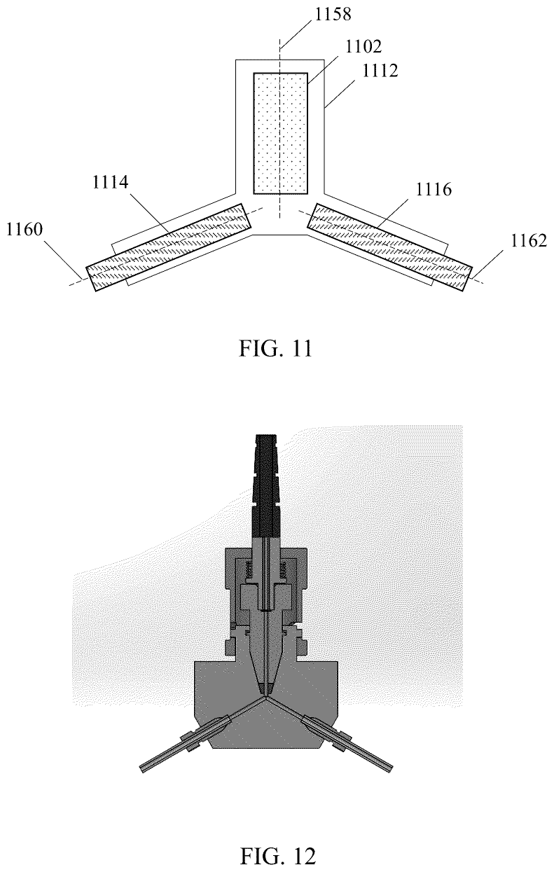

In some embodiments, an instrument comprises a probe, an inlet, and an outlet that are arranged in a manner that assists with preventing the formation of bubbles. It is also possible for an instrument to comprise an article comprising a probe, an inlet, and an outlet arranged in this manner. One such design is shown in FIGS. 11 and 12. FIG. 11 depicts an article comprising a probe, an inlet, an outlet, and a housing. In FIG. 11, the housing 1112 encloses a probe 1102. Additionally, the inlet 1114 is in fluidic communication with the probe 1102 and is configured to provide fluid to the probe. Similarly, the outlet 1116 is in fluidic communication with the probe 1102 and is configured to remove fluid from the probe. As also shown in FIG. 11, the probe 1102 comprises an optical axis 1158, the inlet comprises an inlet flow axis 1160, and the outlet comprises an outlet flow axis 1162. The optical axis may be aligned along a pathway that light may be transmitted through the probe and/or may be aligned along the longest principal axis of the probe. The inlet flow axis may be aligned along the direction that fluid flows through the inlet and/or along the longest principal axis of the inlet. The outlet flow axis may be aligned along the direction that fluid flows through the inlet and/or along the longest principal axis of the outlet. In some embodiments, like the embodiment shown in FIG. 11, a probe, an inlet, and an outlet may be arranged such that the optical, inlet, and outlet axes are positioned in a common plane. It is also possible for the optical axis of the probe to be oriented substantially vertically.

Pumps are a further example of an additional component that can be included in the instruments described herein. As described above, FIG. 9 depicts one example of an instrument comprising a pump. Pumps may be employed at a variety of suitable locations to facilitate fluid flow through the instrument (e.g., upstream of a probe, downstream of a probe, upstream of an inlet, downstream of an outlet). One non-limiting example of a suitable type of pump is a peristaltic pump. Some pumps may have a relatively small size (e.g., they may be miniature pumps).

Another example of an additional component that can be included in the instruments described herein is a temperature control system. The temperature control system may be associated with one or more portion or portions of an instrument, such as with a probe, with a manifold, with tubing fluidically connecting a valve to an inlet and/or to a manifold, with tubing fluidically connecting a valve to a source of a fluid, and/or with a detector (e.g., an optical detector). It is also possible for an instrument to comprise two or more temperature control systems, each of which is associated with a different portion or portions of the instrument. Temperature control systems may be employed to heat and/or to cool the portion(s) of the instrument with which they are associated. Without wishing to be bound by any particular theory, it is believed that cooling a fluid (and/or a sample of a fluid) may advantageously increase the solubility of gases therein, which may reduce bubble formation. Similarly, it is believed that cooling of a detector (e.g., an optical detector) may assist with proper functioning thereof. Non-limiting examples of suitable temperature control systems include a cooling fan, a heat sink, and insulation.

Further examples of additional components that can be included in the instruments described herein are filters, such as purification filters and/or degassing filters. In some embodiments, one or more filters are positioned between a source of samples and a probe (e.g., between a source of samples and an inlet, between a source of samples and a valve, between an inlet and a probe, between a valve and a probe). The filters may remove components of samples of a fluid that would undergo one or more undesirable interactions with the probe, such as impurities (e.g., in the case of a purification filter) and/or gas (e.g., in the case of a degassing filter). Non-limiting examples of impurities include particulates, such as cellular particulates. One example of a purification filter is a filter suitable for alternating tangential flow filtration.

Gas may undesirably be present when a pressurized sample of a fluid is supplied to the instrument from a pressurized additional instrument. A drop in pressure as the sample of the fluid flows to the instrument may result in the formation of bubbles (e.g., microbubbles), which, if they contact the probe, may undesirably affect the signal (e.g., the optical signal) generated. Degassing filters may employ atmospheric pressures and/or pressures below atmospheric pressure to degas samples of fluids.

In some embodiments, an instrument comprises a degasser other than a degassing filter. As an example, in some embodiments, an instrument comprises a vacuum degasser, an ultrasonic degasser, a heater configured to cause degassing, and/or a cooler configured to perform degassing. Vacuum degassers may comprise a gas-permeable membrane and a source of reduced pressure positioned on a side of the permeable membrane opposite the fluid to be degassed. Ultrasonic degassers may comprise a source of ultrasonic waves and a gas sink. A heater configured to cause degassing may comprise a heating element and a gas sink.

In some embodiments, fluid (and/or a sample of a fluid) is passed through a degasser at a relatively higher temperature (e.g., a temperature at which it is received from a source of samples) and then cooled prior to being contacted with a probe. This may advantageously allow for degassing at temperatures at which gas is less soluble and then contact between fluid and the probe at temperatures at which gas is more soluble. Controllers and computers are yet further examples of additional components that can be included in the instruments described herein. In some embodiments, a controller may deliver power and/or instructions to one or more other instrument components (e.g., light source, valve, pump, detector, optical detector). Such instructions may be provided periodically (e.g., pursuant to a pre-selected schedule) and/or on demand (e.g., by an operator). In some embodiments, a controller delivers instructions related to fluid flow (e.g., to start fluid flow, to stop fluid flow, to modify the rate at which fluid flows, to change the location to which fluid flows, to change the location from which fluid flows, to switch the position of a valve). In some embodiments, a controller delivers instructions related to signal (e.g., optical signal) detection (e.g., to turn on a detector and/or an optical detector, to turn off a detector and/or an optical detector, to adjust the manner in which the detector and/or the optical detector performs detection and/or optical detection). Computers may deliver instructions to the controller and/or receive one or more signals from the controller. Controllers and computers may communicate with each other and/or other instrument components in a variety of manners, including via USB cables and/or via ethernet communication.

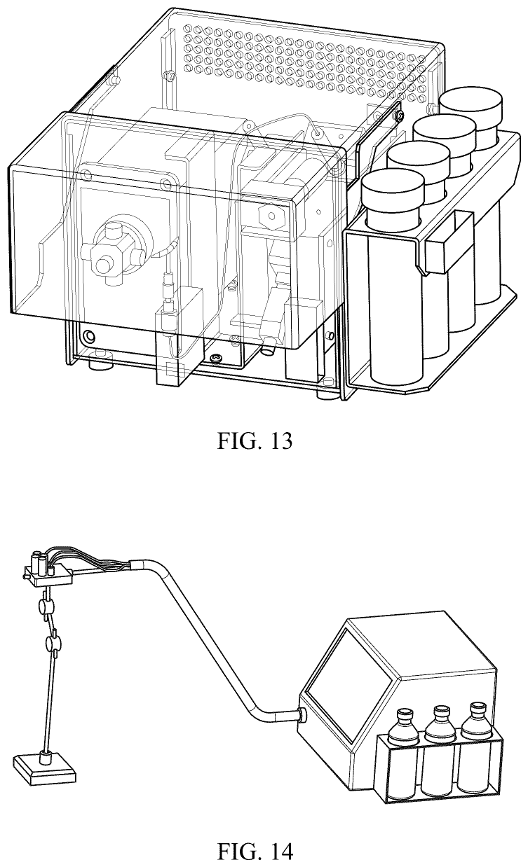

In some embodiments, an instrument is enclosed in an enclosure. As one example, in some embodiments, an instrument is enclosed in an enclosure that comprises a base, a cover, and/or side walls. Some or all of these components may be formed from and/or comprise a light guard. In some embodiments, the enclosure comprises a connector that allows for an optical connection to be formed between one or more portions of the instrument and a device external to the instrument (e.g., an SMA connector, such as an SMA905 connector). Such portions of the instrument may include a light source, an optical detector, and/or a probe. FIG. 13 depicts one non-limiting example of an instrument enclosed by an enclosure.

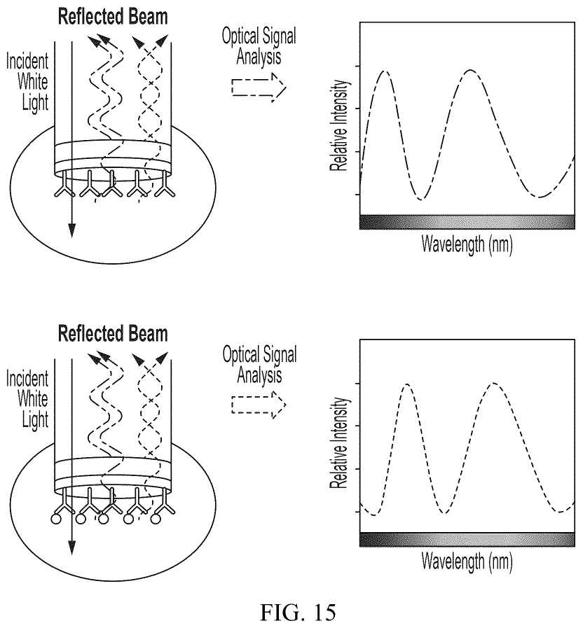

In some embodiments, an instrument comprises two or more parts that are enclosed by separate enclosures. One example of such an instrument is shown in FIG. 14. In FIG. 14, an instrument comprises a first enclosure that encloses a computer, a lamp, and a spectrometer. The first enclosure also includes a display and supports three sources of fluids. The instrument shown in FIG. 14 further comprises a second enclosure that supports a manifold and a probe. The first and second enclosures shown in FIG. 14 are in fluidic, optical, and electrical communication via tubing, one or more optical cables, and one or more electrical cables, respectively. The tubing and the cables shown in FIG. 14 are positioned in a flexible sleeve. In some embodiments, an instrument comprises first and second enclosures that each comprise a connector that allows for an optical connection to be formed (e.g., an SMA connector, such as an SMA905 connector). Such instruments may further comprise an optical cable that is connected optically to both enclosures via these optical connectors. It is also possible for an instrument comprising two or more enclosures to comprise a first enclosure that differs from that shown in FIG. 14 in one or more ways. As an example, in some embodiments, an instrument comprises a first enclosure that, additionally or alternatively, encloses electronics, one or more pumps, a power supply, one or more light sources, and/or one or more detectors (e.g., one or more optical detectors). As another example, an instrument may comprise a first enclosure that comprises a number of sources of fluid other than three (e.g., exactly one source of fluid, exactly two sources of fluid, four or more sources of fluid).

FIG. 14 also depicts an optional mechanical support for the second enclosure. In some embodiments, an instrument comprises a second enclosure that encloses a manifold, one or more probes, one or more valves, a temperature control system, one or more filters (e.g., a degassing filter, a purification filter), and/or one or more pumps. It is also possible for one or more (or all) of these components to be positioned external to any enclosures present in the instrument.

Some instruments described herein may be associated with other instruments. It is also possible for an instrument to be a source of a fluid and/or of one or more samples of a fluid. One example of an instrument is also shown in FIG. 9, in which the instrument is associated with a chromatography column (labeled “CHROM COLUMN” and given reference sign 932). In some embodiments, an instrument described herein is in fluidic communication with an additional instrument. The fluidic communication may be in a manner described elsewhere herein for other sources of fluids and/or sources of samples of a fluid, such as tubing. The association may comprise association that is on-line and/or at-line.

In some embodiments, an instrument described herein is capable of being associated with, configured to be associated with, and/or associated with two or more additional instruments. Similarly, such an instrument may be capable of being in fluidic communication with, configured to be in fluidic communication with, and/or in fluidic communication with two or more additional instruments. In some embodiments, a system comprises both an instrument described herein and an additional instrument. The instruments (e.g., the instrument and the additional instrument) may be of the same type (e.g., two or more bioreactors) or of different types (e.g., a chromatography system and a bioreactor). The association may be reversible. In some such embodiments, the reversible association may be facilitated by the use of a valve that switches between different positions associated with the different additional instruments and/or selects which of the additional instruments is in fluidic communication with and/or supplies a fluid to the instrument comprising the probe.

Some additional instruments may be capable of and/or configured to supply a fluid (and/or one or more samples of a fluid) to the instrument comprising the probe. Some methods may comprise supplying a fluid (and/or one or more samples of a fluid) to the instrument comprising the probe. The fluid may be supplied to the instrument (and/or contacted with a probe) as output by the additional instrument. For instance, the fluid may have the same composition as it did in the additional instrument. It is also possible for the fluid as output by the additional instrument to have a composition that differs from that in the additional instrument by a relatively small amount (e.g., the concentration of each component in the fluid supplied to the instrument and/or contacted with the probe may differ from its concentration in the fluid in the additional instrument by less than or equal to 20%, 10%, 5%, 2%, 1%, 0.5%, 0.2%, or 0.1%; the concentration of each component in the fluid in the additional instrument may differ from its concentration in the fluid supplied to the instrument and/or contacted with the probe by less than or equal to 20%, 10%, 5%, 2%, 1%, 0.5%, 0.2%, or 0.1%). In some embodiments, a fluid that is supplied to the instrument and/or contacted with the probe as output by the additional instrument does not undergo any filtration steps, any purification steps, any centrifugation steps, sterilization steps, and/or any other step that would remove one or more components from the fluid and/or cause a chemical or biological reaction in the fluid after being removed from the additional instrument.

In some embodiments, a fluid is supplied to an instrument from an additional instrument in an automated manner. For instance, the fluid may be supplied from the additional instrument to the instrument without any input by an operator and/or without requiring the operator to perform any steps.

In some embodiments, fluid flowing out of an additional instrument is divided into a plurality of samples prior to flowing into an inlet. The additional instrument may be configured to divide the fluid flowing out of it into the plurality of samples or the instrument comprising the probe may be configured to perform this division.

As described above some embodiments relate to methods. Some methods may be performed partially and/or fully by one or more of the instruments described herein.

Additionally, some instruments may be capable of and/or configured to perform one or more of the methods described herein. An overview of some exemplary methods and steps that may be performed during methods is provided below.

In some embodiments, a method comprises performing one or more steps to determine a concentration of an analyte in a fluid and/or a sample of a fluid. The fluid may be supplied by a bioprocessing system. In some embodiments, such a method further comprises sending instructions to the bioprocessing system based on this concentration. Some methods comprise performing one or more steps to determine the affinity of an analyte for a probe and/or a species immobilized on a probe.

One example of such a step is contacting a probe with a fluid possibly comprising the analyte. The analyte may be present in the fluid at a particular concentration (e.g., a concentration of 0 M, a concentration of higher than 0 M). Upon contact between the fluid and/or sample of a fluid and the probe, some, or all of the analyte present in the fluid and/or sample of the fluid may become immobilized on the probe. The amount of analyte immobilized on the probe may be affected by the amount of analyte present in the fluid and/or sample of the fluid contacting the probe, the amount of analyte already immobilized on the probe, and/or the length of time over which the fluid and/or sample of the fluid and the probe are contacted.

A probe may be contacted with fluids and samples of fluids in a variety of suitable manners. In some embodiments, a probe is contacted with a fluid (and/or a sample thereof) by being positioned in a housing into which fluid (and/or the sample thereof) is introduced in an amount such that contact is made between the fluid (and/or the sample thereof) and the probe. In some embodiments, a probe is contacted with a fluid (and/or a sample thereof) by being positioned in a location that the fluid (and/or the sample thereof) flows over. Contact may be made with the whole probe or with some portions but not others. As an example, in some embodiments, a portion of a probe on which one or more reagents (e.g., one or more reagents on which an analyte may become immobilized) are immobilized is contacted with a fluid and/or a sample of a fluid. As another example, in some embodiments, a portion of a probe that is distal to a portion of the probe that is in contact with an optical cable is contacted with a fluid and/or a sample of a fluid. As a third example, in some embodiments, a face of the probe that is perpendicular to an optical axis of the probe (e.g., the lowermost such face, the face that is perpendicular to an optical axis of the probe and opposite a face contacting an optical cable) is contacted with a fluid and/or a sample of a fluid.

Contact between a fluid (and/or a sample thereof) and a probe may occur over a variety of suitable periods of time. In some embodiments, the time may be relatively short (e.g., seconds to minutes. It is also possible for the period of time over which the probe contacts the fluid to be relatively long (e.g., up to an hour, several hours, longer). Further details regarding the ranges of time over which a probe may contact a fluid are provided below.

The concentration of an analyte in a fluid (and/or a sample thereof) may be relatively constant over a period of time that it contacts a probe, or it vary over that time. As an example of the latter, in some embodiments, analyte becoming immobilized on the probe may be removed from the fluid (and/or the sample thereof) as it is immobilized on the probe. As another example of the latter, in some embodiments, a fluid (and/or a sample thereof) may flow across a probe over a period of time. Different portions of the fluid (and/or the sample thereof) may have different concentrations of analyte therein and so the concentration of the analyte in the fluid contacting the probe may change as different portions of the fluid (and/or the sample thereof) sequentially flow across the probe. In some embodiments, a relatively small amount of analyte is immobilized on a probe relative to the total amount of the analyte in the fluid (and/or the sample thereof) contacting the probe. In such embodiments, the concentration of the analyte in the fluid may be relatively constant over the period of time that the probe contacts the fluid. Similarly, a fluid (and/or a sample thereof) flowing over the probe may have a relatively uniform concentration of the analyte therein.

In some embodiments, a method comprises contacting a probe with a plurality of fluids and/or a plurality of samples of a fluid. The plurality of samples of the fluid may be supplied by a source of samples. The probe may contact the fluids sequentially and/or in an alternating manner. As an example, in some embodiments, a probe is contacted with a plurality of samples of a fluid (e.g., supplied by a source of samples), and, in between samples in the plurality of samples of the fluid, is contacted with one or more fluids that are not samples of the fluid. The fluids that are not samples of the fluid may assist with removing analyte (e.g., analyte originating from a sample) immobilized on the probe in between exposure to different samples of the fluid.

In some embodiments, the fluid to which the probe is exposed may be controlled by the position of a valve with which the probe is in fluidic communication and/or by which valves with which the probe is in fluidic communication with are opened. As an example, in some embodiments, a valve may be switched between positions that place the probe in fluidic communication with different sources of fluids. Switching such a valve from one position to another may remove one source of fluid from fluidic communication with the probe and place another source of fluid in fluidic communication with the probe. As one example, in some embodiments, a valve may be switched to remove a source of samples from fluidic communication with the probe and place a source of a regeneration fluid in fluidic communication with the probe. As another example, in some embodiments, a valve may be switched to remove the source of the regeneration fluid from fluidic communication with the probe and place a source of a neutralization fluid in fluidic communication with the probe. As a third example, in some embodiments, a valve may be switched to remove the source of the neutralization fluid from fluidic communication with the probe and place the source of samples in fluidic communication with the probe.

Some valves described herein may be switched between an open position and a closed position. Switching the valve from the closed position to the open position may place a probe with which the valve is in fluidic communication in fluidic communication with a source of a fluid. Switching the valve from the open position to the closed position may remove a probe with which the valve is in fluidic communication from fluidic communication with a source of fluid. Some methods may comprise opening one or more valves and closing one or more valves to place and remove, respectively, sources of fluids in and from fluidic communication with a probe.

Another example of a step that may be performed during a method described herein is detecting a signal, such as an optical signal. The signal (e.g., the optical signal) may be associated with a fluid (and/or a sample of a fluid) contacting the probe. For instance, the signal (e.g., the optical signal) may be detected at a point in time during which the fluid (and/or the sample of the fluid) contacts the probe. As another example, the signal may be an optical signal that comprises interference between light that has been reflected from two or more interfaces, such as an interface internal to the probe (e.g., between an interior portion of the probe and a coating disposed thereon), between the probe and an analyte immobilized on the probe that was initially present in the fluid (and/or the sample of the fluid) and/or an interface between the analyte and an environment external to the probe (e.g., the fluid, the sample of the fluid). In some embodiments, a signal (e.g., an optical signal) is affected by the amount of analyte immobilized on the probe. Accordingly, analysis of a signal (e.g., an optical signal) may be employed to determine the amount of analyte immobilized on the probe and/or the concentration of analyte present in the fluid and/or sample of the fluid contacted with the probe.

Optical signals may comprise light and/or light interference (e.g., interference between light supplied by a common light source but traveling through optical pathways having different optical thicknesses) or the absence of such interference. As two examples, optical signals may comprise interference between light that is reflected from two different interfaces associated with a probe and/or an analyte immobilized on a probe (e.g., an interface between an interior portion of a probe and a coating disposed on the internal portion of the probe, an interface between the analyte and the probe, an interface between the analyte and an environment external to the probe, an interface at the end of the probe) or the absence of such interference. Light that is reflected from an interface may be supplied to the probe from a light source. As described above, a light source may be optically coupled to a probe such that light is transmitted from the light source and across the probe (e.g., parallel to an optical axis of the probe). Upon reaching an end of the probe, the light may be transmitted out of the probe and/or may reflect from an interface between the probe and an environment external to the probe (and/or from the end of the probe). If there is any analyte immobilized on the probe, some light may reflect from the interface between the probe and the analyte and/or some light may be transmitted through the analyte. The analyte may also change the effective refractive index at the tip and/or change the effective optical path length of the light transmitted through the probe. Light transmitted through the analyte will then encounter the environment with which the analyte is in contact (e.g., a fluid contacting the probe, a sample of a fluid contacting the probe). Some light encountering this environment may be transmitted into the environment with which the analyte is in contact (e.g., an environment external to the probe) and/or may reflect from the interface between the environment and the analyte.

It is also possible for probe described herein to have one or more internal interfaces at which reflection may occur. For instance, some probes may comprise one or more internal interfaces at which reflection can occur, such as an interface between a coating and an interior portion of the probe on which the coating is disposed.

Light reflected from one or more of the above-described locations (and/or any further locations) may travel back through the probe. If light is reflected from multiple locations (e.g., at an interface between the probe and analyte immobilized on the probe, at an interface between analyte immobilized on the probe and an environment external to the probe, at an interface between a coating disposed on an interior portion of the probe and analyte immobilized on the probe, at an interface between an interior portion of the probe and a coating disposed thereon, from the end of the probe), such light may interfere which each other. Light interference may cause the intensity of the interfered light to be higher or lower depending on whether the interference is positive or negative, which may depend on the phase shift between the multiple sources of interfering light. The phase shift may depend on the differences in the path lengths traveled by the light prior to interfering, the refractive index of the material(s) through which the light passes prior to interfering, and/or on the wavelength of light. Accordingly, obtaining information about the intensity of interfered light across a variety of wavelengths may provide information about the thickness of a layer comprising an analyte immobilized on a probe and/or the refractive index of such a layer. This information may be employed to determine the amount of the analyte immobilized on the probe.

FIG. 15 depicts schematically one example of a process by which the amount of analyte immobilized on a probe can be detected. As shown in FIG. 15, light that travels down a probe may reflect from an interface between a coating disposed on an interior portion of a probe and from an interface between analyte disposed on the probe and an environment external to the probe. The phase shift between these two sources of reflected light may depend on the amount of analyte immobilized on the probe and on the wavelength of the reflected light, which may affect the intensity of the reflected light measured. Analysis of the intensity of the reflected light as a function of wavelength may therefore be employed to determine an amount of analyte immobilized on the probe.

It is also possible for an optical signal to comprise a signal other than light interference. As one example, in some embodiments, an optical signal comprises fluorescent light, such as fluorescent light emitted from a species immobilized on a probe and/or generated from a species immobilized on a probe (e.g., via a reaction with a species present in a fluid contacting the probe). As another example, in some embodiments, an optical signal comprises reflected light. For instance, the amount of light reflected at different angles, different angular ranges, and/or over a restricted angular range may be detected. The intensity of such light at one or more particular angles, the intensity of such light over one or more different angular ranges, and/or the angle(s) at which the intensity of such light is lower may be employed to detect the angle at which surface plasmon resonance occurs. This angle may be affected by the immobilization of an analyte on the probe.

In some embodiments, a mechanical signal is detected. As one non-limiting example, in some embodiments, a variation in the resonance of a quartz crystal resonator is a signal that is detected.

Measurements of an amount of analyte immobilized on a probe may be performed on a signal (e.g., an optical signal) that is detected over time. The period of time over which the signal (e.g., the optical signal) may be detected may be the same period of time over which the probe is contacted with a fluid (and/or a sample thereof) or may be a different period of time (e.g., a subset of that period of time). It is also possible for the period of time to be a period of time over which an analyte is removed from the probe (e.g., during a regeneration step, during a neutralization step). Detecting a signal (e.g., an optical signal) over time may comprise detecting a single value of the signal (e.g., the optical signal) from a measurement that is performed over a period of time (e.g., detecting a value that is the average of the signal and/or optical signal over the period of time) and/or may comprise detecting multiple values of the signal and/or optical signal from different measurements that take place over different (overlapping or non-overlapping) periods of time.

In some embodiments, detecting a signal (e.g., an optical signal) over time comprises detecting its variation over time. The variation may comprise an increase, a decrease, or a lack of variation. In some embodiments, the variation comprises the first derivative of the signal (e.g., the optical signal). The variation in a signal (e.g., an optical signal) over a period of time may be determined from multiple measurements made on a single signal (e.g., a single optical signal) over the period of time that yield multiple values of the signal (e.g., the optical signal) over the period of time. The period of time over which the signal (e.g., the optical signal) is measured may comprise a variety of suitable points in time during analyte immobilization on a probe and/or analyte removal from the probe. For instance, the variation may be measured upon initial contact of the probe with a fluid and/or a sample of a fluid comprising an analyte, upon initial removal of contact between the probe and a fluid and/or a sample of a fluid comprising an analyte, when the amount of the analyte immobilized on the probe is at one or more particular percentages of the amount of analyte that would be immobilized on the probe at steady state, when the amount of the analyte immobilized on the probe is the steady-state value, or at any time in between.

The point in time at which the variation of the signal (e.g., the optical signal) is measured may be selected as desired. In some embodiments, the variation of the signal (e.g., the optical signal) is measured when the amount of analyte immobilized on the probe is greater than or equal to 0%, greater than or equal to 5%, greater than or equal to 10%, greater than or equal to 15%, greater than or equal to 20%, greater than or equal to 25%, greater than or equal to 30%, greater than or equal to 35%, greater than or equal to 40%, or greater than or equal to 45% of the amount of analyte that would be immobilized on the probe at steady state. In some embodiments, the variation of the signal (e.g., the optical signal) is measured when the amount of analyte immobilized on the probe is less than or equal to 50%, less than or equal to 45%, less than or equal to 40%, less than or equal to 35%, less than or equal to 30%, less than or equal to 25%, less than or equal to 20%, less than or equal to 15%, less than or equal to 10%, or less than or equal to 5% of the amount of analyte that would be immobilized on the probe at steady state. Combinations of the above-referenced ranges are also possible (e.g., greater than or equal to 0% and less than or equal to 50%, greater than or equal to 0% and less than or equal to 10%, or greater than or equal to 0% and less than or equal to 5%). Other ranges are also possible.

As described above, the value of a signal (e.g., an optical signal) may be indicative of an amount and/or a type of analyte immobilized on a probe. At equilibrium, the amount of analyte immobilized on a probe may be indicative of its affinity for the probe and/or a species immobilized on the probe. Additionally, the variation of a signal (e.g., an optical signal) over time may be indicative of a rate at which an analyte becomes immobilized on a probe. The rate at which the analyte becomes immobilized on the probe may depend on the amount of analyte in the fluid to which the probe is exposed and/or the interaction between the analyte and the probe. As an example of the latter, the rate at which the analyte becomes immobilized on the probe may depend on the affinity of the analyte for the probe (and/or its surface chemistry and/or a reagent immobilized on the probe) and/or the rate at which an analyte binds to the probe (and/or its surface chemistry and/or a reagent immobilized on the probe). As another example of the latter, the rate at which an analyte Is removed from the probe upon contact with a fluid other than that comprising the analyte (e.g., a different sample of the fluid, a regeneration fluid, a neutralization fluid) may also be indicative of the affinity of the analyte for the probe (and/or its surface chemistry and/or a reagent immobilized on the probe) and/or the rate at which an analyte binds to the probe (and/or its surface chemistry and/or a reagent immobilized on the probe). Accordingly, the methods described herein may be suitable for determining the affinity of an analyte for a probe and/or a species immobilized on a probe. The affinity of an analyte for a probe may be parametrized by its association constant and/or its dissociation constant, as described in further detail below. Without wishing to be bound by any particular theory, it is believed that any particular analyte may bind most rapidly to the probe upon initial contact between the fluid comprising the analyte (and/or a sample thereof) and the probe. Accordingly, it is also believed that measuring a variation in a signal (e.g., an optical signal) may yield information that is more precise and/or may yield information more rapidly when the period of time over which the variation is measured comprises the initial contact between the fluid (and/or a sample thereof) and the probe.

Variation in the signal (e.g., the optical signal) may be indicative of changes in the amount of analyte immobilized on a probe, which itself may be indicative of a measurement that is made before the amount of analyte immobilized on the probe achieves a steady-state value and/or of a changing concentration of an analyte in a fluid contacting the probe and/or a sample of a fluid contacting the probe. In the former scenario, the rate at which the analyte immobilized on the probe approaches its steady-state value may vary with the concentration of the analyte in a fluid (and/or sample of a fluid) with which the probe is contacted, and so may be employed to assess the concentration of the analyte in that fluid and/or sample of a fluid. In some embodiments, the variation of the rate at which the analyte immobilized on the probe approaches its steady-state value varies more than the amount of the analyte immobilized on the probe at steady state. Accordingly, in some embodiments, measuring the rate at which an analyte becomes immobilized on a probe may provide a way to determine an analyte concentration that is more sensitive and/or more rapid than measuring an amount of analyte immobilized on a probe at a final steady-state value.

As described above, it is possible for a fluid and/or a sample of a fluid contacting a probe to have a concentration of an analyte therein that changes over time. In such embodiments, any particular determination of an analyte concentration may be a determination of an average amount of the analyte in the fluid that contacted the probe during the period of time over which the determination was made. Multiple determinations of analyte concentration made on such a fluid and/or such a sample of a fluid may yield different analyte concentrations that are indicative of the different analyte concentrations in the fluid and/or sample of the fluid over time.

In some embodiments, signals and/or a variations in signals are measured continuously. This may be accomplished by, at all times during the continuous signal measurement, contacting fluid supplied to an instrument with a probe therein and measuring a signal. In some embodiments, as described in further detail elsewhere herein, multiple probes may be employed to perform such methods. For instance, a first probe may be employed to measure a first signal and/or a first variation in a signal while a second probe is being prepared to do so. Upon measurement of the first signal and/or first signal variation, the fluid may be contacted with a second probe and a second signal and/or second signal variation may be measured. Such processes may be employed with a plurality of probes such that fluid being supplied by an instrument (e.g., a plurality of samples thereof) is being continually contacted with a probe and signals and/or variations thereof are measured continuously. In such embodiments, the fluid may be supplied in an on-line and/or an at-line manner.

In some embodiments, a signal (e.g., an optical signal) that is detected is compared to a model signal profile. The model signal profile may be the model signal profile associated with a desired and/or expected outcome (e.g., an expected signal profile associated with the presence of analyte in a fluid contacting a probe; an expected signal profile associated with the presence of such analyte at an expected time, such as upon column and/or chromatographic media breakthrough in a column and/or chromatographic media with which the probe is in fluidic communication) or an undesired and/or unexpected outcome (e.g., the presence of bubbles in a fluid contacting a probe, instrument malfunction). Comparing a signal (e.g., an optical signal) to a model signal profile may be employed to assess whether the instrument and/or probe is functioning in a normal manner. As an example, if a signal (e.g., an optical signal) does not match a model signal profile associated with an expected outcome, it may indicate the presence of bubbles in the fluid contacted with the probe and/or an instrument malfunction. On the other hand, if a signal (e.g., an optical signal) does match such a model signal profile, it may indicate that the instrument is functioning normally. As another example, if a signal (e.g., an optical signal) matches a model signal profile associated with the presence of bubbles in the fluid contacted with the probe and/or an instrument malfunction, it may indicate the presence of such bubbles and/or malfunction.

In some embodiments, two or more signals (and/or their variations over time) are detected. The different signals may be associated with different samples of a common fluid and/or different fluids. The former may be beneficial for monitoring changes in one or more properties of the fluid present in the additional instrument from which it is supplied. The latter may be beneficial for monitoring properties of fluids present in different additional instruments. The signals may be of the same type or of different types, and may be measured by the same probe or different probes. Similarly, the signals may be measured over different periods of time or over the same period of time. In some embodiments, a first signal is employed to determine a first concentration of a first analyte in a first fluid and a second signal is employed to determine a second concentration of a second analyte in a second fluid.

Some methods described herein relate to techniques that may be employed to determine a concentration of an analyte in a fluid and/or a sample of a fluid that is flowing over a probe. As described elsewhere herein, some instruments comprise fluid sources, inlets, outlets, pumps, and other components that facilitate flow of fluids (and/or samples thereof) over a probe.

Some methods described herein relate to techniques that may be employed to use a single probe to make two or more measurements. In some embodiments, analyte may be immobilized on a probe while the probe is employed to perform a first measurement. It may be challenging or impossible to perform another measurement with that probe while the analyte remains immobilized thereon. Accordingly, methods to remove analyte from a probe may facilitate the use of the probe for multiple measurements.

In some embodiments, a regeneration fluid may be employed to remove analyte from a probe. As an example, in some embodiments, a probe may be contacted with a regeneration fluid after being contacted with a fluid (and/or a sample thereof) comprising an analyte (and, possibly, after a signal and/or an optical signal associated with the fluid is detected). The regeneration fluid may be configured to cause detachment of some or all (e.g., at least a portion of) any analyte immobilized on the probe, such as analyte immobilized on the probe during contact with the fluid comprising the analyte (and/or a sample thereof) and/or during signal (e.g., optical signal) detection. The regeneration fluid may to cause detachment of some or all (e.g., at least a portion of) any analyte immobilized on the probe in a variety of suitable manners. As one example, in some embodiments, a regeneration fluid is capable and/or configured to dissolve some or all of the analyte immobilized on a probe. As another example, in some embodiments, a regeneration fluid is capable of and/or configured to cause a chemical reaction to occur that severs a bond and/or a binding interaction between an analyte and a probe (e.g., it may decrease the affinity of the analyte for the probe, a surface chemistry of the probe, and/or a reagent immobilized on the probe). As a third example, in some embodiments, a regeneration fluid is capable of and/or configured to decompose an analyte in a manner such that the decomposition products are soluble in the regeneration fluid. As a fourth example, in some embodiments, a regeneration fluid comprises a species that has a higher affinity for the probe (and/or its surface chemistry and/or a reagent immobilized thereon) than the analyte and/or a species that is configured to displace the analyte from the probe.

The regeneration fluid may perform one or more of the above-described processes by causing a conformational change in the analyte and/or a reagent immobilized on the probe to which the analyte is bound. The regeneration fluid may cause this change by changing a chemical and/or physical parameter of the fluid to which the probe is exposed, such as its pH, conductivity, and/or temperature. After contact with the regeneration fluid, a sufficient amount of analyte may be detached from the probe so that the probe can be employed to make another measurement. For instance, in some embodiments, a probe is contacted with a first fluid (or first sample of a fluid) comprising an analyte, then contacted with a regeneration fluid, and then contacted with a second fluid (or a second sample of the first fluid or a sample of the second fluid). Signals (e.g., optical signals) may be detected while and/or after the probe is contacted with the first and/or second fluids (and/or first and second samples of fluid).

Although it is possible for measurements to be made on first and second fluids, it should be understood that references to a “first” fluid do not necessarily imply the existence of a “second” fluid or any other fluids. Similarly, references to a “first” fluid do not preclude the presence of a “second” fluid, a “third” fluid, or further fluids not explicitly mentioned. It should also be understood that references to other “firsts” (e.g., a first sample of a fluid, a first signal, a first optical signal, a first concentration, a first analyte, etc.) neither imply the presence of a “second” item of the relevant species nor preclude the presence of “second” or further such items.

In some embodiments, a probe is contacted in an alternating fashion with a fresh sample in a plurality of samples of a fluid and with a regeneration fluid. In other words, the above-described process may be repeated for a plurality of samples of a fluid. In some embodiments, a probe is contacted with a plurality of fluids (e.g., a fresh sample and a regeneration fluid, the two preceding fluids and a neutralization fluid) in a repeating cycle. Repeating the above-described process may allow for the concentration of an analyte in a fluid to be monitored over time. For instance, in the case where signals (e.g., optical signals) associated with some or all of the samples in the plurality of samples are detected and the concentration of the analyte in the sample associated with each signal (e.g., each optical signal) is determined. This may be desirable when it is anticipated that the concentration of an analyte in a fluid may change over time and/or may change as a fluid is sampled. In some embodiments, the above-described process is performed in a manner such that the concentration of an analyte in a fluid is determined repeatedly for a pre-defined period of time, is determined until the concentration exceeds a pre-defined bound (e.g., a pre-defined minimum concentration, a pre-defined maximum concentration, a pre-defined concentration range), and/or is determined until a variation of a signal (e.g., an optical signal) is in excess of a pre-defined bound (e.g., a pre-defined minimum amount, a pre-defined maximum amount, a pre-defined range).

Some regeneration fluids may be well-suited for removing analyte from probes but may leave a probe and/or an instrument in which a probe is positioned in a state that is disadvantageous for further measurements. As an example, in some embodiments, a regeneration fluid may have a pH that would undesirably affect the analyte and/or one or more species present in the fluid (and/or a sample thereof) in which the analyte is positioned. As another example, in some embodiments, a regeneration fluid may comprise a species that would undesirably affect the analyte and/or one or more species present in the fluid (and/or a sample thereof) in which the analyte is positioned. Such a species may be deposited on the probe surface concurrently with and/or after analyte removal therefrom. Contacting a subsequent fluid (or a subsequent sample of a fluid) with the probe may cause exposure of the fluid (and/or a sample thereof) to such undesirable species. Similarly, if contacting the fluid and/or the sample of the fluid with the probe comprises flowing the fluid (and/or the sample thereof) across the probe directly after the regeneration fluid and/or in a manner that displaces the regeneration fluid from the probe, the fluid (and/or the sample thereof) may contact the regeneration fluid and be exposed to such undesirable species.

To address the above-described concerns, a neutralization fluid may be employed. In some embodiments, a probe is contacted with a neutralization fluid after being contacted with a regeneration fluid and/or before being contacted with a second fluid and/or second sample of a fluid. The neutralization fluid may be configured to remove any undesirable species deposited on a probe (and/or a housing in which a probe is positioned) from a regeneration fluid (and/or a second fluid and/or second sample of a fluid) and/or to remove some or all of a regeneration fluid (and/or a second fluid and/or second sample of a fluid) from a housing in which a probe is positioned. It is also possible for a regeneration fluid to adjust one or more physical and/or chemical properties of the probe (e.g., pH, conductivity, and/or temperature).

As described above, FIG. 9 shows one non-limiting example of an embodiment in which an instrument comprises both a source of a neutralization fluid and a source of a regeneration fluid. As also shown therein, a valve may be employed upstream of an inlet to a housing containing a probe that is configured to switch between the source of the regeneration fluid and the source of the neutralization fluid (e.g., to switch between placing the source of the regeneration fluid in fluidic communication with the probe and placing the source of the neutralization fluid in fluidic communication with the probe). That same valve may also be employed to switch between positions associated with those fluids and one or more position(s) associated with one or more sources of an additional fluid (e.g., a fluid possibly comprising an analyte, a fluid to be analyzed in the instrument).

Some methods described herein relate to techniques that may be employed to use multiple probes (e.g., a plurality of probes) together. Probes in a plurality of probes may be arranged in series and/or in parallel. As described above, in some embodiments, it may be desirable for one or more probes to be contacted with a sequence of fluids (e.g., a fluid to be analyzed and a regeneration fluid, the two preceding fluids and a neutralization fluid, multiple samples of a fluid to be analyzed and either or both of a regeneration fluid and a neutralization fluid). When any particular probe is in contact with a fluid that is not a fluid to be analyzed, it may be desirable to still analyze fluids and/or samples from a fluid. Such measurements may be facilitated by the use of multiple probes.

In some embodiments, a method comprises contacting a first probe with a first sequence of fluids, at least one of which comprises a fluid that is not a fluid to be analyzed. While the first probe is contacted with the fluid that is not a fluid to be analyzed (e.g., a regeneration fluid, a neutralization fluid), a second probe may be contacted with a fluid to be analyzed and/or a sample of a fluid to be analyzed. Similarly, while the second fluid is contacted with a fluid to be analyzed and/or a sample of a fluid to be analyzed, the second probe may be contacted with a fluid that is not a fluid to be analyzed (e.g., a regeneration fluid, a neutralization fluid). In some embodiments, fluids to be analyzed and/or samples of fluids are continually supplied to a plurality of probes. The probe to which any particular fluid or sample of a fluid is provided may be one that has been contacted with a regeneration fluid, contacted with a neutralization fluid, and/or comprises a relatively low and/or zero amount of analyte immobilized thereon. After contacting any particular probe with a fluid to be analyzed and/or a sample of a fluid to be analyzed, it may be subsequently contacted with a regeneration fluid and/or a neutralization fluid, after which it may again be contacted with a fluid to be analyzed and/or a sample of a fluid to be analyzed.

In some embodiments, a plurality of samples of a fluid is supplied to a plurality of probes in a manner such that each sample of the fluid is contacted with a probe and each probe is sequentially contacted with a sample of the fluid. As one example, in some embodiments, the following process may be performed sequentially: first, a first probe may be contacted with a first sample of the fluid; then, the first probe may be contacted with a regeneration fluid and/or a neutralization fluid while a second probe is contacted with a second sample of the fluid; then, the first probe may be contacted with a third sample of the fluid while the second fluid is contacted with the regeneration fluid and/or the neutralization fluid; then, optionally, the first probe may be contacted with a regeneration fluid and/or a neutralization fluid while a second probe is contacted with a fourth sample of the fluid. This process may be repeated and/or performed continuously (e.g., with successively increasing samples of the fluid).

It is also possible for two or more probes to be contacted with a common sample of the fluid at the same time and/or for two or more probes to be contacted with different samples of the fluid at the same time. In some embodiments, while the two or more probes are contacted with a common sample and/or different samples, one or more further probes is not contacted with a sample of the fluid. Such probes may be contacted with a fluid other than a sample (e.g., a regeneration fluid, a neutralization fluid). In some embodiments, two or more probes are contacted with a common sample and/or different samples while one or more probes are regenerated. In some embodiments, a method comprises a period of time during the number of probes in a plurality of probes are contacted with a common sample and/or different samples is higher than the number of probes in the plurality of probes that are not contacted with any sample.

In some embodiments, contacting two or more probes with a common fluid (and/or a common sample of a fluid) may assist with detecting any abnormalities associated with one or more of the probes. As an example, two or more probes may be contacted with a common fluid and two or more signals (e.g., two or more optical signals) associated with the common fluid may be generated and detected, each arising from a different probe. The signals (e.g., optical signals) arising from the different probes may be compared to each other. If they are the same as each other, or within a reasonable margin of error from each other, this could indicate that there are no abnormalities associated with any of the probes. On the other hand, if two of the signals (e.g., two of the optical signals) differ from each other by an amount that is outside of a normal error bound, it could indicate that there is an abnormality associated with one of the probes and/or with the portion of the common fluid (and/or sample thereof) contacting one of the probes. Such abnormalities may include gas bubbles, among others.

As described above, it may be possible for a plurality of probes present in an instrument to only include probes do not differ from one another and/or to only include probes that are capable of and/or configured to detect a common analyte. It is also possible for a plurality of probes to comprise two or more probes that differ from one another in one or more ways. As one example, it is possible for a plurality of probes to comprise two or more probes that are capable of and/or configured to detect different analytes. Such probes may be useful when it is desirable to analyze the concentration of more than one analyte in a fluid (and/or a sample of a fluid) and/or to analyze the concentration of different analytes in different fluids (and/or samples of fluids) that are supplied to the probes in the plurality of probes. In some embodiments, a plurality of probes comprises both two probes (e.g., two or more probes) that do not differ from one another and two probes (e.g., two or more probes) that differ from one another.

Some instruments described herein are capable of and/or configured to output one or more signals. Similarly, some methods described herein comprise outputting one or more signals. Advantageously such signals may provide information as to one or more features of a fluid and/or a sample of a fluid with which a probe is in contact.

A variety of signals may be output by the instruments described herein. In some embodiments, an instrument is capable of and/or configured to output an electrical signal. Similarly, some methods may comprise outputting an electrical signal. The electrical signal may be indicative of an amount of analyte immobilized on a probe and/or of a concentration of an analyte in a fluid and/or a sample of a fluid contacting the probe. As another example, an electrical signal may indicate that an analyte immobilized on a probe and/or present in a fluid (and/or a sample thereof) is in excess of a pre-defined amount. It is also possible for an electrical signal to indicate that an analyte immobilized on a probe and/or present in a fluid (and/or a sample thereof) is in an amount that is above the limit at which the instrument can accurately determine the amount of analyte in the fluid (and/or a sample thereof). In some embodiments, an electrical signal indicates that a variation of a signal associated with the immobilization of an analyte on a probe (e.g., an optical signal, a signal associated with the binding of an analyte to a probe) is in excess of a pre-defined amount.

As described elsewhere herein, some instruments described herein are capable of being, configured to be and/or are associated with and/or in fluidic communication with an additional instrument. Some methods may comprise determining the concentration of an analyte in a fluid supplied by and/or received from the additional instrument and/or in one or more samples of a fluid supplied by and/or received from the additional instrument. Some systems may comprise an additional instrument.

As also described elsewhere herein, it is also possible for the instruments described herein to be capable of and/or configured to output one or more signals. In some embodiments, such signals are transmitted to an additional instrument. As one example, a signal may be transmitted to a computer in electrical communication with an instrument described herein (e.g., via a standard specified in Open Platform Communications). The computer may then display the information to an operator and/or record the information to a file. As another example, a signal may be output to an additional instrument (e.g., an additional instrument with which the instrument is in fluidic communication, an additional instrument with which the instrument is not in fluidic communication). Such signals may comprise instructions to perform an action and/or may instruct the additional instrument to perform an action. In some embodiments, an instrument described herein sends instructions and/or a method comprises sending instructions based on a determination of a concentration of an analyte in a fluid contacting a probe.

Non-limiting examples of instructions may comprise instructions to take no action, to halt, to pause, to modify one or more properties of the fluid in the additional instrument, to alter the flow of fluid flowing out of and/or within the additional instrument, to supply the fluid to a different location, and/or to provide fluid flowing out of the additional instrument to a different receptacle (e.g., a different housing containing a different probe, a different inlet, a waste receptacle, a container to temporarily contain the fluid, another column and/or chromatographic media present in the instrument). It should be noted that the fluid may undergo one or more processes when present in the additional instrument that modify one or more of its properties (e.g., the concentration of one or more species therein). Such processes may occur upstream from the probe but downstream from the location of a fluid subject to instructions sent to an additional instrument. Accordingly, instructions sent to an additional instrument on the basis of a concentration of an analyte in a fluid supplied by and/or received from the additional instrument may comprise modifying one or more properties of a fluid present in an additional instrument and differing in one or more ways from the fluid in which the concentration was measured, altering the flow of a fluid flowing out of and/or within the additional instrument and differing in one or more ways from the fluid in which the concentration was measured, supplying a fluid differing in one or more ways from the fluid in which the concentration was measured to a different location, and/or to providing fluid flowing out of the additional instrument and differing in one or more ways from the fluid in which the concentration was measured to a different receptacle. As one example, a measurement may be made of a concentration of an analyte in a fluid supplied by a column and/or chromatographic media in a chromatography system may be the basis for instructions sent regarding whether or not fluid upstream of the column and/or chromatographic media should be continued to be supplied to the column and/or chromatographic media and/or supplied to another column and/or chromatographic media. The fluid upstream of the column and/or chromatographic media may differ from the fluid measured in one or more ways, such as the concentration of the analyte therein (e.g., the concentration of the analyte may be much lower after passing through the column and/or chromatographic media than upstream from the column and/or chromatographic media).

Taking no action may comprise continuing to operate the additional instrument in the manner in which it was operating prior to the sending of the instructions (e.g., supplying the flow from the additional instrument at the same rate and to the same location, maintaining the properties of the fluid which is the same as the fluid contacting the probe or different therefrom in the additional instrument constant, etc.).

Modifying one or more properties of the fluid in the additional instrument may comprise modifying the temperature, pressure, flow rate, pH, conductivity, dissolved oxygen content, and/or concentration of one or more species (e.g., nutrients, including sugars such as glucose, amino acids, and/or fatty acids; and/or metabolites such as lactate) of such a fluid. As noted above, this fluid may be the same as the fluid contacting the probe or may differ therefrom in one or more ways. In some embodiments, modifying one or more properties of a fluid in an additional instrument may be performed as part of a process intended to achieve desired critical quality attributes by providing appropriate critical process parameters. For instance, modifying one or more properties of a fluid in an additional instrument may comprise achieving and/or maintaining desirable cultivation conditions, enhancing one or more features of a product produced by a bioprocess occurring in the additional instrument (e.g., yield, productivity, concentration of the product), and/or reducing product- and/or process-related impurities.

Supplying the fluid to a different location may comprise supplying the fluid to a different probe, to a different location instead of the probe (e.g., to a column and/or chromatographic media in a column or other chromatography system, to a waste receptacle, to a valve, to an outlet, to an analytical system), to the same probe but a different location downstream from the probe (e.g., to a different column and/or chromatographic media in a column or other chromatography system, to a waste receptacle instead of a column and/or chromatographic media in a column or other chromatography system or back to the bioprocessing system, to a column and/or chromatographic media in a column or other chromatography system instead of a waste receptacle, back to the bioprocessing system instead of to a waste receptacle, to a valve, to an outlet, to an analytical system), to the same probe but after passing through a different location upstream from the probe (e.g., a different column, chromatographic media, combination of columns, and/or combination of chromatographic media present in a chromatography system). The different location to which the fluid may be supplied may be a different bioprocessing system or a different location within the same bioprocessing system. As noted above, this fluid may be the same as the fluid contacting the probe or may differ therefrom in one or more ways.

Pausing may comprise pausing one or more operations of the bioprocessing system and/or pausing supply of the fluid to probe. The pausing may occur for a defined period of time and/or may be indefinite (e.g., until an operator triggers a restart, a shutdown, a change in one or more operations, a change in one or more properties of the fluid in the bioprocessing system, and/or a change in the location to which the fluid is supplied). In some embodiments, the pausing may allow for one or more internal adjustments to be made to a bioprocessing system supplying fluid to a probe, to stop a bioprocessing system from supplying a fluid to a probe, to cause a new bioprocessing system to supply a fluid to a probe, and/or to switch the fluid that is supplied to the probe from that supplied by one bioprocessing system to that supplied by another.

In some embodiments, an instrument described herein is capable of and/or configured to interface with an additional instrument in a manner that does not negatively affect the functioning (or, in some embodiments, affect the functionality at all) of the additional instrument. As an example, in some embodiments, an instrument is configured to receive and/or capable of receiving a fluid and/or a sample of a fluid from an additional instrument in a manner preserves the sterility of the fluid remaining in the additional instrument.

In some embodiments, an instrument described herein is capable of and/or configured to interface with an additional instrument that is capable of and/or configured to perform a bioprocess, and/or in which a bioprocess is being performed, such as a bioprocessing system. In such embodiments, the first instrument (e.g., the instrument described above) may be capable of and/or configured to monitor the bioprocess and/or report the results of a measurement performed on a fluid undergoing the bioprocess and/or obtained from the bioprocessing system but differing from fluid present in one or more parts of the bioprocessing system in one or more ways. The measurement may be performed on a sample of the fluid undergoing the bioprocess and/or a sample obtained from a fluid present in the bioprocessing system but differing from fluid present in one or more parts of the bioprocessing system in one or more ways. In some embodiments, the reporting of the measurement results may have a relatively low time lag (e.g., a time lag sufficiently low to enable bioprocess control).

A bioprocessing system may be a system in which a bioprocess is being performed, in Which a bioprocess is capable of being performed, and/or is configured to perform a bioprocess. Non-limiting examples of bioprocesses include biotechnological and biopharmaceutical processes, such as those involved in the manufacturing of a desired therapeutic bioproduct. Non-limiting examples of suitable therapeutic bioproducts include biologics, vaccines, components for cell or gene therapy, and non-therapeutic bioproducts such as pigments, biofuels and/or nutritional supplements. Bioprocesses can involve the expression of the therapeutic bioproduct by a microbial or mammalian cell, and/or a cell itself may be the desired therapeutic bioproduct. In some embodiments, a desired therapeutic bioproduct can be the result of a cell-free production process based on one or more naturally or unnaturally obtained cell components.

In some embodiments, a bioprocess performed in a bioprocessing system (and/or that a bioprocessing system is capable of performing and/or configured to perform) is an upstream process. Upstream processes may include actions and/or workflows performed during the development, optimization, screening and/or selection of a strain and/or cell line, cell cultivation, the production of the desired product with the cells and/or cell components, and/or processes performed between such actions. The cultivation can be performed in various scales (e.g., μL-1000's of L) using different reactor setups and geometries (e.g., rocking motion, stirred tank, bubble column, fixed bed) by applying different modes of operation (e.g., batch, fed-batch, perfusion, continuous, and/or combinations thereof). The cultivation process may be usually monitored, analyzed, and/or controlled based on different sensor technologies (e.g., soft, electrochemical, biochemical, optical; offline, online, inline, atline). In some upstream processes, after the cultivation of a biological agent, it is purified (e.g., via a downstream process). During purification, the cultivation broth may be separated from the desired product, which can be the cells and/or other expressed components (e.g., monoclonal antibodies, polyketides, enzymes, vaccines). One non-limiting example of a bioprocessing system that may be employed in upstream processes is a bioreactor. Non-limiting examples of suitable bioreactors include batch-fed bioreactors, fed-batch bioreactors, and perfusion bioreactors.

In some embodiments, a bioprocess performed in a bioprocessing system (and/or that a bioprocessing system is capable of performing and/or configured to perform) is a downstream process. Downstream processes may include various techniques and methods for recovery, purification, analysis, and/or characterization of the desired product. Downstream processes may involve cell disruption, sedimentation, centrifugation, precipitation, crystallization, extraction, filtration, adjustment of pH and conductivity of liquids, enzymatic or chemical modification, dilution, buffer exchange, evaporation, adsorption, and/or chromatography. In some embodiments, a downstream process includes analysis and/or characterization steps. Such steps may assist with recovering a purified product that is compliant with one or more quality attributes (e.g., glycosylation patterns of antibodies, concentration of endotoxins). A formulation step, which may involve buffer exchange, drying, freeze-drying or crystallization, may be performed to bring the purified product to a suitable state for storage and distribution before it is filled and packaged. Non-limiting examples of bioprocessing systems that may be employed in downstream processes include chromatography systems (e.g., batch chromatography systems, continuous chromatography systems), filtration systems (e.g., tangential flow filtration systems, ultra/diafiltration systems), centrifugation systems, and centrifuges.

As described elsewhere herein, in some embodiments, an instrument described herein is associated with an additional instrument that comprises a chromatography system and/or is a chromatography system. Chromatography systems may comprise columns and/or chromatographic media (e.g., chromatographic media contained in a column). Non-limiting examples of suitable chromatographic media include resins, membrane adsorbers, and monoliths.

In embodiments involving a chromatography system, the chromatography system and/or one or more components thereof (e.g., a column therein, a chromatographic media therein) may supply fluid to a probe. In some embodiments, a method comprises performing one or more operations on a chromatography system and/or a portion thereof. As one example, in some embodiments, a method comprises loading a column and/or chromatographic media in a chromatography system. As another in some embodiments, a method comprises washing a column and/or chromatographic media in a chromatography system (e.g., after it has supplied fluid to a probe, to prepare it to be eluted, to prepare it to be regenerated). As another example, in some embodiments, a method comprises eluting a column and/or chromatographic media (e.g., after it has been loaded with an analyte, such as after it has been supplied with a fluid comprising the analyte, such as after it has been supplying fluid to a probe). As a third example, in some embodiments, a method comprises regenerating a column and/or chromatographic media (e.g., after it has been eluted, after it has been washed, to prepare it to be loaded with analyte from a fluid).

In some embodiments, a method comprises detecting when an analyte first begins to elute from/flow through a chromatography system, a column, in a chromatography system, and/or a chromatographic media present in a chromatography system (e.g., a chromatographic medium present in a column). The analyte may be a species that it would be desirable to recover from the chromatography system or may be an impurity that it would be desirable to eliminate from a sample of a fluid flowing through a chromatography system. The method may comprise dividing a fluid flowing from a chromatography system (e.g., from a first column therein, from a first chromatographic media therein) into a plurality of samples and then contacting each sample with a regenerated probe. Before any analyte is detected in the samples of the fluid, the samples of the fluid may be flowed to a first location after contacting the probe (e.g., a receptacle in which it can be stored, a waste receptacle). Upon initial analyte detection, the chromatography system may adjust the flow such that fluid supplied thereto and/or fluid upstream of the first column and/or chromatographic media (and, possibly, differing in one or more ways from the fluid downstream of the first column, the fluid downstream of the first chromatographic media, and/or the fluid contacted with the probe) is supplied to a second column and/or second chromatographic media instead of the first column and/or chromatographic media. At this point in time, the instrument may continue to be employed to determine an amount of analyte in the samples of the fluid eluting from the chromatography system as a whole (e.g., samples of the fluid that have flowed through the second column and/or second chromatographic media).

In some embodiments, the above-described process is repeated multiple times. As an example, in some embodiments, a chromatography system comprises three or more columns and/or chromatographic media. After breakthrough of the second column and/or chromatographic media is detected in samples of fluid received from the second column and/or chromatographic media, the chromatography system may adjust the flow such that fluid supplied thereto and/or fluid upstream of the second column and/or second chromatographic media is supplied to a third column and/or third chromatographic media instead of the second column and/or second chromatographic media. At this point in time, the instrument may continue to be employed to determine an amount of analyte in the samples of the fluid eluting from the chromatography system as a whole (e.g., samples of the fluid that have flowed through the third column and/or third chromatographic media). In some embodiments, during periods of time during which fluid is not supplied to the first column and/or first chromatographic media (e.g., when it is supplied instead to the second and/or third columns and/or chromatographic media), the first column and/or first chromatographic media may be washed, eluted, and/or regenerated. The above-described process may be repeated with more than one column and/or chromatographic media (e.g., four or more columns and/or chromatographic media) and/or may be performed such that samples of fluid flowing out of a column are provided to the three or more columns and/or chromatographic media in a repeating sequence. The above-described process may be accomplished with the use of counter-current chromatography and/or simulated moving bed chromatography. This may allow for the performance of a continuous chromatography process and, possibly, as a result, an integrated continuous bioprocess (ICB). For example, it may allow for connecting a continuous USP with a continuous DSP, as described herein.

Advantageously, the above-described process may be capable of compensating for variations in the amount of analyte supplied to the chromatography system (e.g., when the fluid is supplied by another bioprocessing system, such as a bioreactor, variations in the analyte produced therein) and/or may compensate for variations in column and/or chromatographic media capacity. Both of these features may reduce analyte loss during purification thereof and/or may be suitable for enhancing usage of resin present in the columns and/or chromatographic media (e.g., enhancing dynamic binding capacity).

In some embodiments, samples of a fluid contacted with a probe may be recycled by the chromatography system. Such samples may be samples that comprise an analyte and/or samples that lack the analyte. The instruments described herein may be employed with a variety of suitable chromatography systems and at a variety of suitable locations in a chromatography process. As one example, in some embodiments, an instrument described herein may be employed to detect column and/or chromatographic media breakthrough, such as breakthrough of a species bound to a column and/or chromatographic media (i.e., transport out of a column and/or chromatographic media of a species loaded thereon). In such embodiments, the concentration of an analyte in samples of a fluid flowing through a column and/or chromatographic media may be determined in order to assess when and/or whether there is any breakthrough. As another example, in some embodiments, an instrument described herein may be employed to detect the elution of an analyte from a column and/or chromatographic media. In such embodiments, the concentration of an analyte in samples of a fluid flowing through a purification column and/or chromatographic media may be determined to assess when elution begins and when elution ends. As further examples, an instrument described herein may be employed to determine the binding capacity of a column and/or chromatographic media for an analyte and/or to generate breakthrough curves for analytes.

One non-limiting example of a suitable chromatography system is a continuous simulated moving bed chromatography system. A variety of suitable columns may be employed, such as capture columns, purification columns, polishing columns, cation exchange columns, anion exchange columns, affinity columns, hydrophobic interaction columns, and/or mixed mode columns. The columns may comprise a chromatography media, such as a resin, a membrane absorber, and/or a monolith.

As also described elsewhere herein, in some embodiments, an instrument described herein is associated with an additional instrument that comprises a bioreactor and/or is a bioreactor. In some embodiments, a method comprises detecting the concentration of an analyte over time in a bioreactor. Advantageously, such methods may be performed without an additional cell removal step prior to the detection of analyte concentration. A sample of the fluid present in the bioreactor may be obtained therefrom and then contacted with the probe. After contact with the probe, the sample of the fluid may be returned to the bioreactor or may be provided to a waste receptacle. The former scenario may advantageously allow for small-scale (e.g., mL-scale) bioreactors to be sampled repeatedly without consuming the entire working volume of the bioreactor. In some embodiments, the sample of the fluid is received from the bioreactor in a manner that preserves the sterility of the fluid remaining in the bioreactor. It is also possible for the sample of the fluid to be received from the bioreactor and pass through the instrument comprising the probe in a manner that preserves its sterility (e.g., so that it can be returned to the bioreactor while still sterile).

It is also possible for an additional instrument to comprise a vessel that receives fluid from one or more of the above-described instruments (e.g., a surge vessel).

Samples of the fluid may be contacted with the probe as frequently as desired (e.g., every few hours) in order to determine a rate of production of an analyte. It is also possible for this process to be employed to determine when the concentration of an analyte is above a pre-defined value and/or a variation of a signal associated with analyte immobilization on a probe (e.g., an optical signal) is in excess of a pre-defined amount. For instance, a method may comprise determining when the concentration of an analyte is at a level that indicates that growth in the bioreactor can be terminated and the desired product (e.g., the analyte) may be obtained therefrom. As another example, a method may comprise determining when the concentration of an analyte is at a level that is undesirable for cell growth. In such cases, the method may comprise outputting a signal indicating this fact, which may automatically trigger an adjustment in the growth conditions and/or alert an operator to take action.

In some embodiments, an instrument described herein is configured to be reversibly associated with, capable of being reversibly associated with, and/or reversibly associated with two or more additional instruments, such as two or more bioprocessing systems and/or two or more bioreactors. Such instruments may be configured to be in fluidic communication with, capable of being in fluidic communication with, and/or in fluidic communication with the two or more additional instruments, bioprocessing systems, and/or bioreactors. In some embodiments, the association and/or fluidic communication is reversible. For instance, the association and/or fluidic communication may occur through a single inlet. In such embodiments, a valve may be employed to switch positions between positions that place the probe in fluidic communication with the two or more bioreactors. The use of two or more bioreactors to supply a single inlet may be advantageous when it is desirable to determine the concentration of the analyte in the additional instruments, bioprocessing systems, and/or bioreactors at intervals that are relatively infrequent in comparison to the amount of time required to determine the concentration of the analyte and/or regenerate the probe. In such embodiments, a single instrument may advantageously be used to monitor the concentration of an analyte in the two or more additional instruments, bioprocessing systems, and/or bioreactors over time, requiring less instrumentation to perform these measurements than would be needed if each bioreactor was associated with a different instrument.

Similarly, some methods comprise contacting a probe with fluids supplied from two or more additional instruments and some systems may comprise two or more additional instruments, each configured to supply and/or capable of supplying the instrument with fluid (e.g., a first fluid output from a first additional instrument and a second fluid output from a second additional instrument).

In some embodiments, the two or more additional instruments are two or more bioprocessing systems. As one example, in some embodiments, a method comprises contacting a probe with fluids supplied from two bioprocessing systems, a system comprises two bioprocessing systems, and/or an instrument is configured to be reversibly associated with, capable of being reversibly associated with, and/or reversibly associated with two or more bioprocessing systems. In some embodiments, one of the bioprocessing systems is an upstream bioprocessing system and one of the bioprocessing systems is a downstream bioprocessing system. As one example, in some embodiments, one of the bioprocessing systems comprises a bioreactor and one of the bioprocessing systems comprises a chromatography system. In such embodiments, it may be possible for a method to comprise employing a bioreactor to perform a bioprocess that results in the generation of an analyte and also comprise employing a chromatography system to capture the analyte. This may be performed in an automated manner and/or without operator input. For instance, based on measurements of the concentration of the analyte in fluid supplied by the bioreactor, instructions may be sent to the bioreactor to continue to perform the bioprocess and/or to supply fluid to the chromatography system. As another example, based on measurements of the concentration of the analyte in fluid supplied by the chromatography system, instructions may be sent to the chromatography system to continue to load a column and/or chromatographic media therein, to elute a column and/or chromatographic media, and/or to direct fluid (e.g., supplied by the bioreactor, supplied by a column and/or chromatographic media in the chromatography system) to a particular column and/or chromatographic media. In some embodiments, a common probe may be employed to perform all such concentration measurements, possibly without the need for threshold adjustment.

It is also possible for two or more of the bioprocessing systems to be upstream bioprocessing systems and/or two or more of the bioprocessing systems to be downstream bioprocessing systems.

In some embodiments in which two or more additional instruments are present, supply fluid, are capable of supplying fluid, and/or configured to supply fluid, a detector present in an instrument may be capable of detecting and/or configured to detect signals when the probe contacted with the fluids supplied by each additional instrument. For instance, the detector may be configured to detect and/or capable of detecting a variation of a first signal when the probe is contacted with the fluid supplied by the first additional instrument and also configured to detect and/or capable of detecting a variation of a second signal when the probe is in contact with the fluid supplied by the second additional instrument.

In some embodiments in which two or more additional instruments are present, supply fluid, are capable of supplying fluid, and/or configured to supply fluid, an instrument and/or system may be capable of sending and/or configured to send instructions to one of the additional instruments on the basis of a concentration of an analyte in a fluid supplied by another additional instrument. Similarly, some methods comprise determining the concentration of an analyte in a fluid supplied by a first additional instrument and sending instructions to a second additional instrument on the basis of this determination. Such instructions may comprise those described above (e.g., modifying one or more properties of the second fluid in the bioprocessing system, continuing to supply the second fluid to the probe, supplying the second fluid to a different location, and/or taking no action). As non-limiting examples, in some embodiments, instructions may be sent to a chromatography system based on a concentration of an analyte in a fluid supplied by a bioreactor, instructions may be sent to a bioreactor based on a concentration of an analyte in a fluid supplied by a chromatography system, and/or instructions may be sent to one chromatography system based on the concentration of an analyte in a fluid supplied by the first chromatography systems (e.g., to supply the fluid to a different location, such as a different column and/or chromatographic media in the chromatography system, to recycle the fluid, to supply the fluid to a waste receptacle).

Non-limiting further examples of additional instruments that may be in fluidic communication with the instruments described herein include filtration devices, centrifuges, pumps, and valves.

As described above, in some embodiments, an instrument comprises a probe. The probe may assist with the detection of a concentration of an analyte in a fluid and/or sample of a fluid contacting the probe.

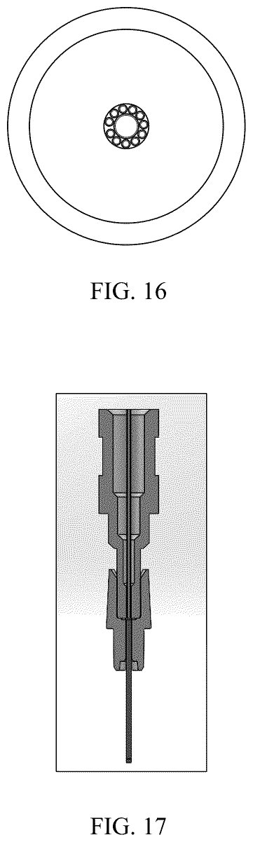

The probes described herein may have a variety of suitable designs. In some embodiments, the probe is an optical probe. As one example, a probe may be a fiber-optic probe and/or comprise an optical fiber. FIG. 16 shows one non-limiting embodiment of a cross-section of a probe comprising an optical fiber.

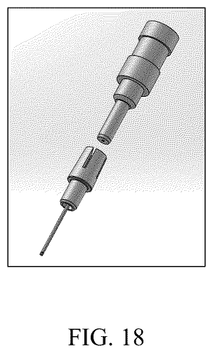

In some embodiments, a probe comprises one or more components that allow it to be optically coupled to an optical cable. As an example, in some embodiments, a probe comprises a component, such as a plastic hub, that is compatible with an SMA connector (e.g., an SMA905 connector), a BNC connector, a connector with push, lock, and/or twist functionality, and/or a compression spring. FIGS. 17-18 show one example of such a component in combination with an optical cable. In some embodiments, a probe is coupled to an optical cable via a ferrule. The ferrule may comprise optical fibers comprising polished tips, which may facilitate optical communication with the probe. The optical cable may comprise one or more components to assist with strain relief at the location of the coupling.

In some embodiments, a probe is transparent to and/or may transmit light at a plurality of wavelengths (e.g., visible wavelengths, near infrared wavelengths). This may be facilitated by, in some embodiments, the presence of one or more polished ends (e.g., polished ends that are perpendicular to the optical axis of the probe).

In some embodiments, a probe comprises a surface that is functionalized and/or that has a surface chemistry that assists with the immobilization of an analyte. For instance, the surface functionalization and/or chemistry may promote the bonding of one or more analytes thereto. In some embodiments, a probe comprises a surface on which one or more reagents (e.g., one or more reagents suitable for immobilizing an analyte) are immobilized. The reagent(s) may be immobilized on the probe in a variety of suitable manners. As an example, the reagent(s) may be bonded to the probe. The bonding may comprise covalent bonding, ionic bonding, polar bonding, van der Waals bonding, hydrophobic bonding, and/or hydrogen bonding.

In some embodiments, one or more reagents(s) may be immobilized on the probe in a manner such that they do not undergo significant (and/or any) detachment from the probe upon contact with a fluid to be analyzed (and/or a sample thereof), contact with a regeneration fluid, contact with a neutralization fluid, and/or any other process typically performed during one or more of the above-described methods. For instance, the reagent(s) may be immobilized on the probe in a manner that is stable to water, aqueous solutions, buffers, acids, bases, and/or bodily fluids.

Additionally or alternatively, it is possible for one or more reagent(s) to be immobilized on a probe in a manner such that the probe can be refunctionalized. It is also possible for a method to comprise refunctionalizing a probe. Refunctionalization may comprise removing one or more reagent(s) from the probe. For instance, in some embodiments, refunctionalization comprises exposing a probe on which one or more reagent(s) are immobilized to a fluid (e.g., a buffer, such as an acidic buffer) that causes one or more of those reagent(s) to be detached from the probe. Afterwards, the probe may be exposed to a fluid comprising one or more new reagent(s) to be immobilized on the probe. Refunctionalizing a probe may advantageously allow a probe to be employed during more than one method and/or to immobilize more than one type of analyte. As an example, a first reagent and/or set of reagents may be immobilized on the probe prior to the determination of the concentration of a first analyte in a fluid and/or a sample of a fluid. In some circumstances, it may be desirable to reuse the probe to determine the concentration of a second analyte in a fluid and/or a sample of the fluid (e.g., the same fluid as before, a different fluid, the same sample as before, a different sample). Accordingly, after detection of the concentration of the first analyte, the probe may be refunctionalized to yield a probe onto which a new reagent and/or set of reagents can be immobilized, thus allowing for the probe to be employed to detect the concentration of the second analyte.

It is also possible for some probes to not be refunctionalized and/or to be incapable of refunctionalization.

A variety of suitable reagents may be immobilized on the surfaces of the probes described herein. Some reagents may be species that are capable of that engaging in one or more chemical reactions (e.g., one or more chemical reactions that result in the immobilization of an analyte on a probe). For instance, a probe may comprise a reagent that is capable of bonding with an analyte (e.g., covalently, ionically, by polar interactions, by van der Waals interactions, hydrophobically, by hydrogen bonding, by complexing), absorbing an analyte, and/or adsorbing an analyte. In some embodiments, one or more of the previously described chemical reactions may cause the analyte with which the reagent reacts to become immobilized thereon. Selected non-limiting examples of suitable reagents include biomolecules (e.g., proteins, glycoproteins, peptides, nucleic acids (e.g., DNA, RNA, mRNA), antibodies (e.g., antibodies for exosomes, such as anti-CD63 and/or anti-CD9, antibodies for proteins, antibodies for viruses, antibodies for virus-like particles), antibody fragments, antigens, polysaccharides, carbohydrates, hormones, streptavidin, glutathione), ligands (e.g., ligands for proteins, such as protein A), small molecules, viruses, cells, inorganic compounds (e.g., aminopropylsilane), sequestration compounds, capsids, bacteria, resins (e.g., Ni-NTA), plasmids, nutrient components, metabolics, metabolic byproducts, and combinations thereof. Non-limiting examples of proteins include protein A, protein G, protein L, and lectin. One non-limiting example of a combination of two or more of the previously described reagent types is a reagent that comprises protein A and an antibody to an exosome and/or a virus. The antibody may be immobilized on protein A immobilized on a probe surface and may be capable of immobilizing an exosome and/or a virus. In such embodiments, as well as others, two or more reagents are immobilized on a probe (and, in some embodiments, one or more such reagents may be a combination of two or more reagents).

In some embodiments, a reagent immobilized on a surface of a probe is suitable for engaging in a chemical and/or biological reaction that comprises binding. It is also possible for a probe to be suitable for engaging in a chemical and/or biological reaction that does not comprise binding. When present, binding may comprise a reaction between a target and a binding partner that specifically binds to the target (e.g., an agent or molecule that specifically binds to the target). Binding may also comprise immobilizing a target (e.g., an analyte) on the binding partner. In some embodiments, the binding partner may specifically bind to an epitope on the target molecule (e.g., an analyte). Non-limiting examples of specific pairs of binding partners and targets include an antibody and an antigen, an antibody fragment and an antigen, an antibody and a hapten, an antibody and a peptide, an antibody and a small molecule, an antigen and a fusion protein, an antibody fragment and a hapten, an enzyme and an enzymatic substrate, an enzyme and an inhibitor, an enzyme and a cofactor, a binding protein and a substrate, a carrier protein and a substrate, a protein and a small molecule, lecithin and a carbohydrate, a receptor and a hormone, a receptor and an effector, complementary strands of nucleic acid, a protein in combination with a nucleic acid repressor and an inducer, a ligand and a cell surface receptor, a virus and a ligand, and a receptor and a ligand.

Non-limiting examples of antibodies that may be binding partners or antibodies include intact (i.e., full-length) polyclonal and monoclonal antibodies, antigen-binding fragments of polyclonal and monoclonal antibodies (such as Fab, Fab′, F(ab′)2, or Fv), single chains (scFv), mutants of single chains, fusion proteins comprising an antibody portion, humanized antibodies, chimeric antibodies, diabodies, linear antibodies, single chain antibodies, multispecific antibodies (e.g., bispecific antibodies), and modified configurations of the immunoglobulin molecule that comprise an antigen recognition site of the required specificity. Non-limiting examples of antibodies falling into the last category include glycosylation variants of antibodies, amino acid sequence variants of antibodies, and covalently modified antibodies. Additionally, a binding partner may be an antibody of any class, such as IgD, IgE, IgG, IgA, or IgM (or sub-class thereof, e.g., IgG1, IgG2, IgG3, IgG4, IgA1 and/or IgA2.

An antigen may be a molecule or a portion of a molecule that can have antibodies generated against it. Antigens may be peptides, polysaccharides and/or lipids. Some antigens may originate from within the body (a “self-antigen”), and some antigens may originate from the external environment (a “non-self-antigen”).

In some embodiments, antibodies suitable for performing a chemical and/or biological reaction specifically bind to epitopes on their target molecules. An epitope (which may be referred to as an antigenic determinant) may be the part of the antigen recognized (or bound by) an antibody. For example, the epitope may be the specific piece of the antigen to which an antibody binds. The part of an antibody that binds to the epitope may be referred to as a paratope. An epitope may be a conformational epitope (composed of discontinuous amino acids or sections of the antigen) or a linear epitope (composed of continuous amino acids). Some proteins may share segments of high sequence homology and/or structural similarity. These similar proteins may have common epitopes (in other words, the epitopes on different antigens may be bound by the same antibody). Further, a protein that has been processed differentially (such as a protein that has gone a further enzymatic process) may share some, but not all epitopes with its pre-processing form. Non-limiting examples of different epitopes that may be added or removed during processing include N-terminal signal peptides (as seen, for example, on pre-pro-peptides) and changes seen when an inactive protein (e.g., a pro-peptide) is turned into an active form by post-translational modification.

When an antibody specifically binds to an epitope, it may engage in a binding reaction that is capable of discriminating between a target molecule (e.g., an analyte) and a non-target molecule (e.g., a molecule other than the analyte of interest). For example, a binding partner may specifically bind to a target molecule with greater than or equal to 2-fold greater affinity than to a non-target molecule with greater than or equal to 4-fold, greater than or equal to 5-fold, greater than or equal to 6-fold, greater than or equal to 7-fold, greater than or equal to 8-fold, greater than or equal to 9-fold, greater than or equal to 10-fold, greater than or equal to 20-fold, greater than or equal to 25-fold, greater than or equal to 50-fold, or greater than or equal to 100-fold greater affinity than to a non-target molecule.

The binding affinity of an antibody may be parametrized by its affinity (K_{D}). The K_{D} is the ratio of the dissociation constant to the association constant (K_{D}−K_{d}/Kₐ). In some embodiments, a binding partner described herein has an affinity (K_{D}) of less than or equal to 10⁻⁵ M, less than or equal to 10⁻⁶ M, less than or equal to 10⁻⁷ M, less than or equal to 10⁻⁸ M, less than or equal to 10⁻⁹ M, less than or equal to 10⁻¹⁰ M, less than or equal to 10⁻¹¹ M, or less than or equal to 10⁻¹². An increased affinity K_{D} corresponds to a decreased dissociation constant K_{d} or an increased association constant (Kₐ). Higher affinity binding of a binding partner (e.g., an antibody) to a first molecule relative to a second molecule can be indicated by a higher Kₐ (or a smaller numerical value of K_{D} and/or K_{d}) for binding to the first target than the Kₐ (or numerical value of K_{D} and/or K_{d}) for binding to the second target. In such cases, the antibody has a higher specificity for the first molecule (e.g., a protein in a first conformation or mimic thereof) relative to the second molecule (e.g., the same protein in a second conformation or mimic thereof, or a second protein). Differences in binding affinity (e.g., specificity) can be greater than or equal to 1.5-fold, greater than or equal to 2-fold, greater than or equal to 3-fold, greater than or equal to 4-fold, greater than or equal to 5-fold, greater than or equal to 10-fold, greater than or equal to 15-fold, greater than or equal to 20-fold, greater than or equal to 37.5-fold, greater than or equal to 50-fold, greater than or equal to 70-fold, greater than or equal to 80-fold, greater than or equal to 90-fold, greater than or equal to 100-fold, greater than or equal to 500-fold, greater than or equal to 1000-fold, greater than or equal to 10,000-fold, greater than or equal to 105-fold.

In some embodiments, a reagent may be immobilized on a surface of a probe via a covalent bond. Prior to such immobilization, the surface of the probe may be functionalized such that it comprises a plurality of functional groups suitable for forming such covalent bonds. For instance, the surface of the probe may be functionalized by reaction with a bifunctional reagent comprising a siloxane group that facilitates attachment to the probe and a functional group that facilitates the formation of a covalent bond with the reagent to be immobilized on the probe. As another example, the surface of the probe may be exposed to a plasma or other treatment that generates functional groups in situ that facilitate the formation of a covalent bond with the reagent to be immobilized on the probe. Non-limiting examples of suitable types of functionals group that facilitate the formation of a covalent bond with the reagent to be immobilized on the probe include hydroxyls, amines, and carboxyls.

The probes described herein may be formed from a variety of suitable materials and/or comprise a coating formed from a variety of suitable materials. In some embodiments, a probe comprises a glass and/or a polymer and/or a coating comprising a glass and/or a polymer. Non-limiting examples of suitable glasses include SiO₂ and Ta₂O₅. Non-limiting examples of suitable polymers include polystyrene and polyethylene.

The probes described herein may comprise optical fibers having a variety of suitable diameters. In some embodiments, a probe comprises an optical fiber having a core with a diameter of greater than or equal to 400 microns, greater than or equal to 500 microns, greater than or equal to 600 microns, greater than or equal to 700 microns, greater than or equal to 800 microns, greater than or equal to 900 microns, greater than or equal to 1000 microns, greater than or equal to 1100 microns, greater than or equal to 1200 microns, greater than or equal to 1300 microns, greater than or equal to 1400 microns, greater than or equal to 1500 microns, greater than or equal to 1600 microns, greater than or equal to 1700 microns, greater than or equal to 1800 microns, or greater than or equal to 1900 microns. In some embodiments, a probe comprises an optical fiber having a core with a diameter of less than or equal to 2000 microns, less than or equal to 1900 microns, less than or equal to 1800 microns, less than or equal to 1700 microns, less than or equal to 1600 microns, less than or equal to 1500 microns, less than or equal to 1400 microns, less than or equal to 1300 microns, less than or equal to 1200 microns, less than or equal to 1100 microns, less than or equal to 1000 microns, less than or equal to 900 microns, less than or equal to 800 microns, less than or equal to 700 microns, less than or equal to 600 microns, or less than or equal to 500 microns. Combinations of the above-referenced ranges are also possible (e.g., greater than or equal to 400 microns and less than or equal to 2000 microns). Other ranges are also possible.

A variety of suitable detectors may be employed in the instruments described herein. In some embodiments, an instrument comprises an optical detector. Non-limiting examples of suitable types of optical detectors include photon-counting devices, spectrophotometers, spectrometers (e.g., Raman spectrometers, infrared spectrometers), polarization detectors, photodiodes, CCD/CMOS sensors, and imaging sensors. Such optical detectors may be configured to and/or capable of detecting a variation of an optical signal over one or more periods of time. For instance, such optical detectors may be able to make relatively rapid measurements of an optical signal and/or measure an optical signal over a relatively short period of time. It is also possible for some optical detectors to be configured to and/or capable of detecting a plurality of optical signals (e.g., a plurality of optical signals, each associated with a fluid and/or a sample of a fluid). In some embodiments, an optical detector is configured to and/or capable of detecting the intensity of light as a function of position (which, in turn, may allow for the detection of the intensity of light as a function of the angle from which it reflected from the probe) and/or detecting the intensity of light across a restricted angular range.

In some embodiments, an instrument comprises a mechanical detector, such as a quartz crystal microbalance.

As described above, in some embodiments, an instrument comprises a light source. The light source may serve as a source of light that stimulates the emission of an optical signal.

In some embodiments, a light source supplies light at a plurality of wavelengths. For instance, an instrument may comprise a light source that comprises an incandescent bulb. As further examples, an instrument may comprise a light source that comprises a lamp, such as a halogen lamp, a xenon lamp, a mercury lamp, an LED, and/or an arc lamp. When an instrument comprises a light source supplies light at a plurality of wavelengths, the instrument may further comprise one or more optical filters. Such optical filter(s) may be positioned between the light source and the location of the species generating the optical signal and/or between the light source and the detector. The former may be beneficial if the light emitted by the light source comprises at least one wavelength that would stimulate the generation of an optical signal other than those desired (e.g., in the case where light at the relevant wavelength would stimulate emission from a variety of species, including a species that would always be immobilized on the probe and/or present in a fluid and/or a sample thereof contacting the probe). It is also possible for a light source to supply light over a restricted wavelength range (e.g., the light source may comprise a laser or other narrow-band source).

The light sources described herein may also supply light at a single polarization and/or at a plurality of polarizations. When a light source supplies light at a plurality of polarizations, the instrument may further comprise one or more polarizing filters. Such polarizing filter(s) may be positioned between the light source and the location of the species generating the optical signal and/or between the light source and the detector.

In some embodiments, a light source supplies light at a variety of (e.g., all or substantially all) angles. It is also possible for a light source to supply light over a restricted angular range.

As described above, in some embodiments, the amount of an analyte in a fluid and/or a sample of a fluid is determined. Further detail regarding possible fluids, samples of fluids, and analytes is provided below.

In some embodiments, a fluid (and/or a sample thereof) possibly comprises an analyte. Some methods may comprise determining whether such a fluid (and/or a sample of a fluid) actually comprises the analyte and/or the amount in which the fluid (and/or a sample of the fluid) comprises the analyte. Accordingly, some fluids and/or samples of fluids may comprise an analyte (in a variety of suitable amounts) and some fluids and/or samples of fluids may lack an analyte. Additionally, it should also be understood that references to an analyte concentration in a fluid (and/or a sample of a fluid) encompass concentrations that are identically zero and encompass concentrations that are greater than zero.

The fluids described herein may comprise a variety of suitable analytes, non-limiting examples of which include proteins (e.g., protein A, protein G, protein L, host cell proteins, Fc receptors, streptavidin), peptides, antibodies (e.g., IgG), antigens, small molecules, viruses, capsids, cells (e.g., Chinese hamster ovary cells), differentiated cell types, polysaccharides, bacteria, nucleic acids (e.g., DNA, RNA, mRNA), exosomes, extracellular vesicles, and ions (e.g., nickel ions). In some embodiments, a fluid comprises an analyte that is a tagged protein, such as a protein tagged by a recombinant modification. Non-limiting examples of tagged proteins include His-tagged proteins and biotin-tagged proteins.

Analytes may be labeled or unlabeled. Unlabeled analytes may lack a label that facilitates detection (e.g., a label that facilitates optical detection, a label that is fluorescent, etc.), may have the same chemical composition (e.g., the same chemical formula) as they do in an additional instrument from which they are supplied, and/or may have the same chemical composition as they do once purified to form a final product. In some embodiments, advantageously, a method comprises determining the concentration of an unlabeled analyte in a fluid and/or an instrument is configured to determine and/or capable of determining the concentration of an unlabeled analyte in a fluid. This may allow for facile label-free detection, and/or may allow for analyte concentration and/or properties to be determined without the need for a labeling step and/or without the influence of an attached label.

The fluids and samples of fluids described herein may comprise an analyte at a variety of suitable concentrations. In some embodiments, a fluid and/or a sample of a fluid comprises an analyte at a concentration of greater than or equal to 0.000001 g/L, greater than or equal to 0.000002 g/L, greater than or equal to 0.000005 g/L, greater than or equal to 0.0000075 g/L, greater than or equal to 0.00001 g/L, greater than or equal to 0.00002 g/L, greater than or equal to 0.00005 g/L, greater than or equal to 0.000075 g/L, greater than or equal to 0.0001 g/L, greater than or equal to 0.0002 g/L, greater than or equal to 0.0005 g/L, greater than or equal to 0.00075 g/L, greater than or equal to 0.001 g/L, greater than or equal to 0.002 g/L, greater than or equal to 0.005 g/L, greater than or equal to 0.0075 g/L, greater than or equal to 0.01 g/L, greater than or equal to 0.02 g/L, greater than or equal to 0.05 g/L, greater than or equal to 0.075 g/L, greater than or equal to 0.1 g/L, greater than or equal to 0.2 g/L, greater than or equal to 0.5 g/L, greater than or equal to 0.75 g/L, greater than or equal to 1 g/L, greater than or equal to 1.5 g/L, greater than or equal to 2 g/L, greater than or equal to 2.5 g/L, greater than or equal to 3 g/L, greater than or equal to 3.5 g/L, greater than or equal to 4 g/L, greater than or equal to 4.5 g/L, greater than or equal to 5 g/L, greater than or equal to 6 g/L, greater than or equal to 7.5 g/L, greater than or equal to 10 g/L, greater than or equal to 15 g/L, greater than or equal to 20 g/L, greater than or equal to 30 g/L, or greater than or equal to 40 g/L. In some embodiments, a fluid and/or a sample of a fluid comprises an analyte at a concentration of less than or equal to 5 g/L, less than or equal to 50 g/L, less than or equal to 40 g/L, less than or equal to 30 g/L, less than or equal to 20 g/L, less than or equal to 15 g/L, less than or equal to 10 g/L, less than or equal to 7.5 g/L, less than or equal to 6 g/L, less than or equal to 5 g/L, less than or equal to 4.5 g/L, less than or equal to 4 g/L, less than or equal to 3.5 g/L, less than or equal to 3 g/L, less than or equal to 2.5 g/L, less than or equal to 2 g/L, less than or equal to 1.5 g/L, less than or equal to 1 g/L, less than or equal to 0.75 g/L, less than or equal to 0.5 g/L, less than or equal to 0.2 g/L, less than or equal to 0.1 g/L, less than or equal to 0.075 g/L, less than or equal to 0.05 g/L, less than or equal to 0.02 g/L, less than or equal to 0.01 g/L, less than or equal to 0.0075 g/L, less than or equal to 0.005 g/L, less than or equal to 0.002 g/L, less than or equal to 0.001 g/L, less than or equal to 0.00075 g/L, less than or equal to 0.0005 g/L, less than or equal to 0.0002 g/L, less than or equal to 0.0001 g/L, less than or equal to 0.000075 g/L, less than or equal to 0.00005 g/L, less than or equal to 0.00002 g/L, less than or equal to 0.00001 g/L, less than or equal to 0.0000075 g/L, less than or equal to 0.000005 g/L, or less than or equal to 0.000002 g/L. Combinations of the above-referenced ranges are also possible (e.g., greater than or equal to 0.000001 g/L and less than or equal to 50 g/L). Other ranges are also possible.

In some embodiments, a fluid and/or a sample of a fluid comprises additional species in addition to possibly comprising an analyte. For instance, as described above, in some embodiments a fluid and/or a sample of a fluid is supplied by an additional instrument. In such embodiments, the fluid and/or the sample of the fluid may further comprise one or more species that facilitate the use of the additional instrument and/or are present during a process being performed in the additional instrument. As one example, in some embodiments, a fluid to be analyzed is a crude sample and/or a sample of a fluid to be analyzed is a crude sample. As an additional example (e.g., in the case where the additional instrument is a chromatography system), a fluid and/or a sample of a fluid comprises a buffer. As a third example (e.g., in the case where the additional instrument is a bioreactor, the fluid and/or the sample of the fluid comprises one or more components of a cell media, non-limiting examples of which include glucose, lactate, amino acids (e.g., of one or more types), salts (e.g., of one or more types), proteins (e.g., of one or more types, such as protein A, protein G, protein L, host cell proteins), peptides, one or more types of nucleic acids, and/or one or more types of cells. It is also possible for a fluid and/or a sample of a fluid to have one or more features that are helpful and/or necessary for operation of the additional instrument, such as being sterile.

Some fluids and/or samples of fluids may comprise bodily fluids and/or biological materials. In some embodiments, a fluid and/or a sample of a fluid comprises an analyte that is a biological material (e.g., located in a biological fluid, located in a buffer). As an example, in some embodiments, a fluid and/or a sample of a fluid comprises, as an analyte and/or as a species other than an analyte, cells (e.g., live cells) and/or reagents (e.g., biomolecules). Fluids and/or samples of fluids may comprise some or all of the reagents described elsewhere herein with respect to the reagents that may be immobilized on the surface of a probe and/or may comprise reagents other than those so described. Such reagents may be analytes to be detected or may be present in fluids and/or samples of fluids possibly comprising such analytes. Non-limiting examples of such reagents include proteins, glycoproteins, peptides, ligands, antibodies, antigens, hormones, nucleic acids (e.g., DNA, RNA), polysaccharides, carbohydrates, small molecules, inorganic compounds, sequestration compounds, viruses, extracellular vesicles, exosomes, capsids, cells, differentiated cell types, and bacteria.

It is also possible for a measurement to be performed on a fluid that is a standard. For instance, in some embodiments, a measurement is performed on a positive standard and/or a negative standard. Positive standards may be configured to always yield a signal (e.g., an optical signal) and/or to always yield a known signal (e.g., a known optical signal) if the instrument is performing correctly. Some positive standards comprise a known concentration of an analyte in a fluid. Negative standards may be configured to always yield a signal (e.g., an optical signal) indicative of no analyte immobilization of the instrument is performing correctly. Some negative standards lack any analyte. The performance of a method on a positive standard and/or a negative standard may be useful for calibrating the results obtained from an instrument and/or to confirm that an instrument is performing properly. Some embodiments may comprise contacting a probe with a plurality of fluids that comprises a fluid and/or a sample of a fluid to be analyzed (and, optionally, lacks a standard) and a second probe with a plurality of fluids that comprises a standard (and, optionally, lacks a fluid and/or a sample of a fluid to be analyzed). It is also possible for an embodiment to comprise contacting two or more standards (e.g., with two or more probes, in a manner such that each probe contacts a single standard, in a manner such that one probe contacts both standards, in a manner such that both probes contact both standard), such as both a positive standard and a negative standard and/or two or more positive standards comprising differing concentrations of an analyte.

In some embodiments, a fluid and/or samples of a fluid are supplied from a source of a fluid that is in fluidic communication with an instrument described herein by tubing that is relatively short. The tubing may have a length of greater than or equal to 0.1 cm, greater than or equal to 0.2 cm, greater than or equal to 0.5 cm, greater than or equal to 0.75 cm, greater than or equal to 1 cm, greater than or equal to 2 cm, greater than or equal to 5 cm, greater than or equal to 7.5 cm, greater than or equal to 10 cm, greater than or equal to 12.5 cm, greater than or equal to 15 cm, greater than or equal to 17.5 cm, greater than or equal to 20 cm, greater than or equal to 22.5 cm, greater than or equal to 25 cm, greater than or equal to 27.5 cm, greater than or equal to 30 cm, greater than or equal to 32.5 cm, greater than or equal to 35 cm, greater than or equal to 37.5 cm, greater than or equal to 40 cm, greater than or equal to 42.5 cm, greater than or equal to 45 cm, greater than or equal to 47.5 cm, greater than or equal to 50 cm, greater than or equal to 55 cm, greater than or equal to 60 cm, greater than or equal to 75 cm, greater than or equal to 1 m, greater than or equal to 2 m, greater than or equal to 5 m, or greater than or equal to 7.5 m. The tubing may have a length of less than or equal to 10 m, less than or equal to 7.5 m, less than or equal to 5 m, less than or equal to 2 m, less than or equal to 1 m, less than or equal to 75 cm, less than or equal to 60 cm, less than or equal to 55 cm, less than or equal to 50 cm, less than or equal to 47.5 cm, less than or equal to 45 cm, less than or equal to 42.5 cm, less than or equal to 40 cm, less than or equal to 37.5 cm, less than or equal to 35 cm, less than or equal to 32.5 cm, less than or equal to 30 cm, less than or equal to 27.5 cm, less than or equal to 25 cm, less than or equal to 22.5 cm, less than or equal to 20 cm, less than or equal to 17.5 cm, less than or equal to 15 cm, less than or equal to 12.5 cm, less than or equal to 10 cm, less than or equal to 7.5 cm, less than or equal to 5 cm, less than or equal to 2 cm, less than or equal to 1 cm, less than or equal to 0.75 cm, less than or equal to 0.5 cm, or less than or equal to 0.2 cm. Combinations of the above-referenced ranges are also possible (e.g., greater than or equal to 0.1 cm and less than or equal to 10 m, greater than or equal to 5 cm and less than or equal to 2 m, greater than or equal to 5 cm and less than or equal to 50 cm, or greater than or equal to 10 cm and less than or equal to 50 cm). Other ranges are also possible.

In some embodiments, a fluid and/or samples of a fluid are supplied from a source of a fluid that is in fluidic communication with an instrument described herein by tubing that has a relatively low in-line volume. The tubing may have an in-line volume of greater than or equal to 10 microliters, greater than or equal to 20 microliters, greater than or equal to 30 microliters, greater than or equal to 40 microliters, greater than or equal to 50 microliters, greater than or equal to 80 microliters, greater than or equal to 100 microliters, greater than or equal to 200 microliters, greater than or equal to 500 microliters, greater than or equal to 800 microliters, greater than or equal to 1 mL, greater than or equal to 2 mL, greater than or equal to 5 mL, greater than or equal to 8 mL, greater than or equal to 10 mL, greater than or equal to 20 mL, greater than or equal to 30 mL, greater than or equal to 40 mL, greater than or equal to 50 mL, greater than or equal to 80 mL, greater than or equal to 100 mL, greater than or equal to 125 mL, greater than or equal to 150 mL, or greater than or equal to 175 mL. The tubing may have an in-line volume of less than or equal to 200 mL, less than or equal to 175 mL, less than or equal to 150 mL, less than or equal to 125 mL, less than or equal to 100 mL, less than or equal to 80 mL, less than or equal to 50 mL, less than or equal to 40 mL, less than or equal to 30 mL, less than or equal to 20 mL, less than or equal to 10 mL, less than or equal to 8 mL, less than or equal to 5 mL, less than or equal to 2 mL, less than or equal to 1 mL, less than or equal to 800 microliters, less than or equal to 500 microliters, less than or equal to 200 microliters, less than or equal to 100 microliters, less than or equal to 80 microliters, less than or equal to 50 microliters, less than or equal to 40 microliters, less than or equal to 30 microliters, or less than or equal to 20 microliters. Combinations of the above-referenced ranges are also possible (e.g., greater than or equal to 10 microliters and less than or equal to 200 mL, greater than or equal to 40 microliters and less than or equal to 40 mL, or greater than or equal to 80 microliters and less than or equal to 10 mL). Other ranges are also possible.

As described above, some instruments comprise sources of regeneration fluids and some methods comprise contacting a probe with a regeneration fluid. Further details regarding some suitable regeneration fluids are provided below.

In some embodiments, a regeneration fluid comprises a buffer. It is also possible for the regeneration fluid to comprise a salt (e.g., an acetate salt, such as sodium acetate; a citrate salt, a phosphate salt, a tris buffer salt, sodium hydroxide) and/or an organic molecule (e.g., glycine, biotin, histidin). The salt may assist with buffering the regeneration fluid. In some embodiments, a regeneration fluid comprises a species, such as a small molecule or a salt, that is capable of and/or configured to bind to an analyte, such as an analyte immobilized on the probe. As one example, a regeneration fluid may comprise biotin to regenerate a probe employed to determine the concentration of a streptavidin analyte. As another example, a regeneration fluid may comprise histidin to regenerate a probe employed to determine the concentration of a nickel ion analyte. It is also possible for a species present in a regeneration fluid to bind to a reagent immobilized on a probe, thereby displacing an analyte immobilized thereon (e.g., biotin in a regeneration fluid may bind to a streptavidin reagent immobilized on the probe, thereby displacing an analyte therefrom).

The regeneration fluids described herein may have a variety of suitable pHs. In some embodiments, a regeneration fluid is acidic. Acidic regeneration fluids may be particularly suitable for probes employed with fluids and/or samples of fluids possibly comprising an analyte that is an antibody, a protein (e.g., protein A, protein G, protein L) and/or a nucleic acid (e.g., DNA, RNA). A regeneration fluid may have a pH of greater than or equal to 1, greater than or equal to 1.5, greater than or equal to 2, greater than or equal to 2.5, greater than or equal to 3, greater than or equal to 3.5, greater than or equal to 4, or greater than or equal to 4.5. A regeneration fluid may have a pH of less than or equal to 5, less than or equal to 4.5, less than or equal to 4, less than or equal to 3.5, less than or equal to 3, less than or equal to 2.5, less than or equal to 2, or less than or equal to 1.5. Combinations of the above-referenced ranges are also possible (e.g., greater than or equal to 1 and less than or equal to 5). Other ranges are also possible. For instance, it is also possible for the regeneration fluid to have a neutral or basic pH.

As described above, some instruments comprise sources of neutralization fluids and some methods comprise contacting a probe with a neutralization fluid. Further details regarding some suitable neutralization fluids are provided below.

In some embodiments, a neutralization fluid comprises a buffer. Non-limiting examples of suitable buffers include phosphate-buffered saline and tris buffers.

The neutralization fluids described herein may have a variety of suitable pHs. In some embodiments, a neutralization fluid has a pH that is neutral or close to neutral. A neutralization fluid may have a pH of greater than or equal to 6, greater than or equal to 6.5, greater than or equal to 7, greater than or equal to 7.5, greater than or equal to 8, greater than or equal to 8.5, or greater than or equal to 9. A neutralization fluid may have a pH of less than or equal to 9, less than or equal to 8.5, less than or equal to 8, less than or equal to 7.5, less than or equal to 7, or less than or equal to 6.5. Combinations of the above-referenced ranges are also possible (e.g., greater than or equal to 6 and less than or equal to 9, or greater than or equal to 6 and less than or equal to 8). Other ranges are also possible.

As described above, in some embodiments, an instrument comprises sources of fluid other than a source of samples, a source of a regeneration fluid, and/or a source of a neutralization fluid. Similarly, in some embodiments, an instrument comprises a valve that comprises one or more positions that are in fluidic communication with sources of fluid other than a source of samples, a source of a regeneration fluid, and/or a source of a neutralization fluid. Non-limiting further examples of sources of fluid include a source of a fluid comprising a primary antibody, a source of a fluid comprising a secondary antibody, a source of a wash buffer, and a source of a fluid comprising a substrate for a secondary antibody. Without wishing to be bound by theory, these sources of fluids may be beneficial when the instrument is employed to form an assay. For instance, a primary antibody present in a fluid that contacts a probe may become immobilized on the probe. Such primary antibodies may be suitable for immobilizing an analyte present in a sample of a fluid. As another example, a secondary antibody present in the fluid that contacts a probe may become immobilized on an analyte immobilized on the probe. The secondary antibody may facilitate detection of the analyte. Similarly, a fluid comprising a substrate for a secondary antibody may facilitate detection analyte by interaction with the secondary antibody. As a third example, a wash buffer may be suitable for removing one or more components from a probe.

One further example of a source of a fluid that may be present in an instrument described herein is a source of a dilutant. The source of the dilutant may be positioned such that it is configured to supply a dilutant to mix with one or more fluids supplied by sources of fluids (e.g., a sample of a fluid). The source of the dilutant may be positioned upstream of a valve that may be capable of supplying, configured to supply, and/or supply the diluted fluid to the probe. The instrument may be configured to mix the dilutant with the relevant fluid. In some embodiments, this mixing may occur upstream from the valve that is capable of supplying, configured to supply, and/or supplies the diluted fluid to the probe. Then, a mixed fluid comprising both the dilutant and the fluid supplied form a source of fluids may flow through the valve and then contact a probe.

As described elsewhere herein, the instruments described herein may comprise microfluidic channels.

The microfluidic channels described herein may have a variety of suitable dimensions perpendicular to fluid flow. Such dimensions may be referred to elsewhere herein as “widths” even if they are oriented vertically. In some embodiments, one or more, or each microfluidic channel in an instrument independently has a width of one or more of the following ranges: greater than or equal to greater than or equal to 0.5 mm, greater than or equal to 0.75 mm, greater than or equal to 1 mm, greater than or equal to 2 mm, greater than or equal to 5 mm, or greater than or equal to 7.5 mm. In some embodiments, each microfluidic channel in an instrument independently has a width of less than or equal to 10 mm, less than or equal to 7.5 mm, less than or equal to 5 mm, less than or equal to 2 mm, less than or equal to 1 mm, or less than or equal to 0.75 mm. Combinations of the above-referenced ranges are also possible (e.g., greater than or equal to 0.5 mm and less than or equal to 10 mm). Other ranges are also possible.

As described above, in some embodiments, a probe is contacted with a fluid and/or sample of a fluid. It is also possible for a probe to be contacted with a plurality of fluids in a repeating cycle, such as a plurality of fluids comprising a plurality of fluids possibly comprising an analyte and/or a plurality of samples of a fluid. Further details regarding such contact are provided below.

Fluids and samples of fluids may be contacted with probes for a variety of suitable periods of time. In some embodiments, contacting a probe with a fluid (and/or a sample thereof) comprises contacting the probe with the fluid (and/or the sample thereof) for a period of time of greater than or equal to 1 second, greater than or equal to 2 seconds, greater than or equal to 5 seconds, greater than or equal to 10 seconds, greater than or equal to 15 seconds, greater than or equal to 20 seconds, greater than or equal to 25 seconds, greater than or equal to 30 seconds, greater than or equal to 40 seconds, greater than or equal to 1 minute, greater than or equal to 2 minutes, greater than or equal to 5 minutes, greater than or equal to 10 minutes, greater than or equal to 20 minutes, greater than or equal to 30 minutes, greater than or equal to 45 minutes, greater than or equal to 1 hour, greater than or equal to 1.5 hours, greater than or equal to 2 hours, greater than or equal to 2.5 hours, greater than or equal to 3 hours, greater than or equal to 3.5 hours, or greater than or equal to 4 hours. In some embodiments, contacting a probe with a fluid (and/or a sample thereof) comprises contacting the probe with the fluid (and/or the sample thereof) for a period of time of less than or equal to 5 hours, less than or equal to 4 hours, less than or equal to 3.5 hours, less than or equal to 3 hours, less than or equal to 2.5 hours, less than or equal to 2 hours, less than or equal to 1.5 hour, less than or equal to 1 hour, less than or equal to 45 minutes, less than or equal to 30 minutes, less than or equal to 20 minutes, less than or equal to 10 minutes, less than or equal to 5 minutes, less than or equal to 2 minutes, less than or equal to 1 minute, less than or equal to 40 seconds, less than or equal to 30 seconds, less than or equal to 25 seconds, less than or equal to 20 seconds, less than or equal to 15 seconds, less than or equal to 10 seconds, less than or equal to 5 seconds, or less than or equal to 2 seconds. Combinations of the above-referenced ranges are also possible (e.g., greater than or equal to 1 second and less than or equal to 5 hours, greater than or equal to 1 second and less than or equal to 1 hour, or greater than or equal to 10 seconds and less than or equal to 30 seconds). Other ranges are also possible.

Repeating cycles in which a plurality of fluids possibly comprising an analyte and/or a plurality of samples of a fluid are contacted with a probe may be performed over a variety of suitable cycle times. In some embodiments, the time to complete a cycle is greater than or equal to 30 seconds, greater than or equal to 40 seconds, greater than or equal to 50 seconds, greater than or equal to 60 seconds, greater than or equal to 70 seconds, or greater than or equal to 80 seconds. In some embodiments, the time to complete a cycle is less than or equal to 90 seconds, less than or equal to 80 seconds, less than or equal to 70 seconds, less than or equal to 60 seconds, less than or equal to 50 seconds, or less than or equal to 40 seconds. Combinations of the above-referenced ranges are also possible (e.g., greater than or equal to 30 seconds and less than or equal to 90 seconds). Other ranges are also possible.

Repeating cycles in which a plurality of fluids possibly comprising an analyte and/or a plurality of samples of a fluid are contacted with a probe may be performed at a variety of suitable intervals. In some embodiments, the interval is greater than or equal to 30 seconds, greater than or equal to 1 minute, greater than or equal to 2 minutes, greater than or equal to 5 minutes, greater than or equal to 10 minutes, greater than or equal to 20 minutes, greater than or equal to 30 minutes, greater than or equal to 45 minutes, greater than or equal to 1 hour, greater than or equal to 1.5 hours, greater than or equal to 2 hours, greater than or equal to 3 hours, greater than or equal to 4 hours, greater than or equal to 6 hours, greater than or equal to 8 hours, greater than or equal to 12 hours, or greater than or equal to 16 hours. In some embodiments, the interval is less than or equal to 24 hours, less than or equal to 16 hours, less than or equal to 12 hours, less than or equal to 8 hours, less than or equal to 6 hours, less than or equal to 4 hours, less than or equal to 3 hours, less than or equal to 2 hours, less than or equal to 1.5 hours, less than or equal to 1 hour, less than or equal to 45 minutes, less than or equal to 30 minutes, less than or equal to 20 minutes, less than or equal to 10 minutes, less than or equal to 5 minutes, less than or equal to 2 minutes, or less than or equal to 1 minute. Combinations of the above-referenced ranges are also possible (e.g., greater than or equal to 30 seconds and less than or equal to 24 hours). Other ranges are also possible.

Repeating cycles in which a plurality of fluids possibly comprising an analyte and/or a plurality of samples of a fluid are contacted with a probe may be performed for a variety of suitable total times. In some embodiments, repeating cycles are performed (e.g., continuously, at a pre-set interval) for a period of time of greater than or equal to 0.5 minutes, greater than or equal to 1 minute, greater than or equal to 2 minutes, greater than or equal to 5 minutes, greater than or equal to 7.5 minutes, greater than or equal to 10 minutes, greater than or equal to 15 minutes, greater than or equal to 20 minutes, greater than or equal to 30 minutes, greater than or equal to 45 minutes, greater than or equal to 1 hour, greater than or equal to 2 hours, greater than or equal to 3 hours, greater than or equal to 6 hours, greater than or equal to 9 hours, greater than or equal to 12 hours, greater than or equal to 15 hours, greater than or equal to 18 hours, greater than or equal to 1 day, greater than or equal to 1.5 days, greater than or equal to 2 days, greater than or equal to 5 days, greater than or equal to 1 week, greater than or equal to 2 weeks, or greater than or equal to 1 month. In some embodiments, repeating cycles are performed for a period of time of less than or equal to 3 months, less than or equal to 1 month, less than or equal to 2 weeks, less than or equal to 1 week, less than or equal to 5 days, less than or equal to 2 days, less than or equal to 1.5 days, less than or equal to 1 day, less than or equal to 18 hours, less than or equal to 15 hours, less than or equal to 12 hours, less than or equal to 9 hours, less than or equal to 6 hours, less than or equal to 3 hours, less than or equal to 2 hours, less than or equal to 1 hour, less than or equal to 45 minutes, less than or equal to 30 minutes, less than or equal to 20 minutes, less than or equal to 15 minutes, less than or equal to 10 minutes, less than or equal to 7.5 minutes, less than or equal to 5 minutes, less than or equal to 2 minutes, or less than or equal to 1 minute. Combinations of the above-referenced ranges are also possible (e.g., greater than or equal to 0.5 minutes and less than or equal to 3 months). Other ranges are also possible.

In some embodiments, a method described herein is carried out with the assistance of a computer and/or a processor, such as via a computer implemented control system. Additionally, some systems and instruments described herein comprise a computer and/or a processor. Such methods, systems, and instruments are not limited in their implementation to any specific computer system described herein, as many other different machines may be used.

The computer implemented control systems described herein can be part of or coupled in operative association with an instrument and/or a system, configured and/or programmed to control and adjust operational parameters of an instrument and/or a system, and/or to analyze, calculate, and/or determine values (e.g., concentrations). In some embodiments, such as when a defined value or threshold is reached, a computer implemented control system can send and receive reference signals to set and/or control operating parameters of an instrument and/or system. In some embodiments, a computer implemented control system can be separate from and/or remotely located with respect to an instrument and/or may be configured to receive data from one or more remote sample instruments via indirect and/or portable means, such as via portable electronic data storage devices, such as magnetic disks, or via communication over a computer network, such as the Internet or a local intranet.

A computer implemented control system may include components and circuitry, such as a processing unit (i.e., a processor), a memory system, input and output devices and interfaces (e.g., an interconnection mechanism), and/or other components, such as transport circuitry (e.g., one or more busses), a video and audio data input/output (I/O) subsystem, special-purpose hardware, and/or other components and circuitry, as described below in more detail. Further, a computer implemented control system may be a multi-processor computer system and/or may include multiple computers connected over a computer network.

A computer implemented control system may include a processor, for example, a commercially available processor such as one of the series x86, Celeron and Pentium processors, available from Intel, similar devices from AMD and Cyrix, the 680X0 series microprocessors available from Motorola, the PowerPC microprocessor from IBM, and ARM processors. Many other processors are available, and the computer system is not limited to a particular processor.

In some embodiments, a processor executes a program called an operating system, of which WindowsNT, Windows95 or 98, Windows 7, Windows 8, UNIX, Linux, DOS, VMS, MacOS and OSX, and iOS are examples, which may control the execution of other computer programs and/or may provide scheduling, debugging, input/output control, accounting, compilation, storage assignment, data management and/or memory management, communication control and/or other related services. The processor and operating system together may define a computer platform for which application programs in high-level programming languages are written. The computer implemented control system is not limited to a particular computer platform.

In some embodiments, a processor is in electronic communication, is capable of being in electronic communication, and/or is configured to be in electronic communication with one or more other components present in a system described herein. For instance, a processor may be in electronic communication with a detector.

In some embodiments, a processor is programmed to perform one or more methods described herein and/or one or more values are determined with the use of a processor. As an example, in some embodiments, a processor is programmed to determine a concentration of an analyte (e.g., a first concentration of a first analyte, a second concentration of a first analyte, a second concentration of a second analyte) in a fluid (e.g., a first fluid, a second fluid). This determination may be performed based on the variation of a signal (e.g., a signal detected by a detector, such as an optical signal detected by an optical detector) over a period of time. As another example, a processor may be programmed to determine whether a threshold has been reached based on the variation of a signal (e.g., a signal detected by a detector, such as an optical signal detected by an optical detector) over a period of time. The threshold may be indicative of a certain value of a first derivative of a signal, such as a value of the first derivative of the signal that is the limit of what the instrument is capable of detecting and/or configured to detect.

A computer implemented control system may include a memory system, which may include a computer readable and writeable non-volatile recording medium, of which a magnetic disk, optical disk, a flash memory and tape are examples. Such a recording medium may be removable, for example, a floppy disk, read/write CD or memory stick, or may be permanent, for example, a hard drive.

Such a recording medium may store signals, typically in binary form (i.e., a form interpreted as a sequence of one and zeros). A disk may (e.g., magnetic or optical) have a number of tracks, on which such signals may be stored, typically in binary form, i.e., a form interpreted as a sequence of ones and zeros. Such signals may define a software program, e.g., an application program, to be executed by the microprocessor, or information to be processed by the application program.

The memory system of the computer implemented control system also may include an integrated circuit memory element, which typically is a volatile, random access memory such as a dynamic random access memory (DRAM) or static memory (SRAM). In operation, a processor may cause programs and data to be read from the non-volatile recording medium into the integrated circuit memory element, which may allow for faster access to the program instructions and data by the processor than the non-volatile recording medium does.

The processor may manipulate the data within the integrated circuit memory element in accordance with the program instructions. Then, it may copy the manipulated data to the non-volatile recording medium after processing is completed. The computer implemented control system is not limited to a particular memory system.

At least part of such a memory system described above may be used to store one or more data structures (e.g., look-up tables) or equations described above. For example, at least part of the non-volatile recording medium may store at least part of a database that includes one or more of such data structures. Such a database may be any of a variety of types of databases, for example, a file system including one or more flat-file data structures where data is organized into data units separated by delimiters, a relational database where data is organized into data units stored in tables, an object-oriented database where data is organized into data units stored as objects, another type of database, or any combination thereof.

The computer implemented control system may include a video and audio data I/O subsystem. An audio portion of the subsystem may include an analog-to-digital (A/D) converter, which may receive analog audio information and convert it to digital information. The digital information may be compressed using known compression systems for storage on the hard disk to use at another time. A video portion of the I/O subsystem may include a video image compressor/decompressor. Such compressor/decompressors may convert analog video information into compressed digital information, and/or vice-versa. The compressed digital information may be stored on hard disk for use at a later time.

The computer implemented control system may include one or more output devices. Example output devices include a cathode ray tube (CRT) display, liquid crystal displays (LCD) and other video output devices, printers, communication devices such as a modem or network interface, storage devices such as disk or tape, and audio output devices such as a speaker. Such output devices may comprise an output interface which may output information to an operator, an instrument, a component of a system, and/or a component of a different system capable of receiving and/or configured to receive such information. In some embodiments, the information may take the form of a signal, such as an electronic signal encoding such information, a visual signal informing an operator of such information, and/or an electronic signal encoding instructions (e.g., instructions supplied to an additional instrument, such as a bioprocessing system, based on the determination of a concentration of an analyte in a fluid). As one example, in some embodiments, an output interface is capable of indicating and/or is configured to indicate a concentration of an analyte in a fluid.

In some embodiments, an output interface comprises a display interface. The display interface may display information to an operator. In some embodiments, displaying information comprises providing a numerical indication of the information on the display interface. For instance, a display interface may indicate a concentration of an analyte in a fluid by providing a numerical indication thereof on the display interface. The display interface may, additionally or alternatively, display other information, such as the status of an instrument, signals (e.g., optical signals indicative of binding, such as binding signals), and/or first derivatives of signals (e.g., first derivatives of optical signals indicative of binding, such as binding signals). Such display interfaces may display information that is contemporaneously obtained (e.g., contemporaneously obtained values of signals and/or first derivatives thereof).

The computer implemented control system also may include one or more input devices. Example input devices include a keyboard, keypad, track ball, mouse, pen and tablet, communication devices such as described above, and data input devices such as audio and video capture devices and sensors. Such input devices may comprise an input interface which may receive information from an operator, an instrument, a component of a system, and/or a component of a different system capable of providing and/or configured to provide such information. In some embodiments, the information may take the form of a signal, such as a signal from which a concentration of an analyte in a fluid may be determined. The information may be received over a network or may be directly input into the input interface (e.g., mechanically).

It should be appreciated that one or more of any type of computer implemented control system may be used to implement various embodiments described herein. Aspects of the invention may be implemented in software, hardware or firmware, or any combination thereof. The computer implemented control system may include specially programmed, special purpose hardware, for example, an application-specific integrated circuit (ASIC). Such special-purpose hardware may be configured to implement one or more of the methods, steps, simulations, algorithms, systems, and system elements described above as part of the computer implemented control system described above or as an independent component.

The computer implemented control system and components thereof may be programmable using any of a variety of one or more suitable computer programming languages. Such languages may include procedural programming languages, for example, C, Pascal, Fortran and BASIC, object-oriented languages, for example, C++, Java and Eiffel and other languages, such as a scripting language or even assembly language.

The methods, steps, simulations, algorithms, systems, and system elements may be implemented using any of a variety of suitable programming languages, including procedural programming languages, object-oriented programming languages, other languages and combinations thereof, which may be executed by such a computer system. Such methods, steps, simulations, algorithms, systems, and system elements can be implemented as separate modules of a computer program, or can be implemented individually as separate computer programs. Such modules and programs can be executed on separate computers.

Such methods, steps, simulations, algorithms, systems, and system elements, either individually or in combination, may be implemented as a computer program product tangibly embodied as computer-readable signals on a computer-readable storage medium, for example, a non-volatile recording medium, an integrated circuit memory element, or a combination thereof. For each such method, step, simulation, algorithm, system, or system element, such a computer program product may comprise computer-readable signals tangibly embodied on the computer-readable storage medium that define instructions (e.g., encoded therein), for example, as part of one or more programs, that, as a result of being executed by a computer, instruct the computer to perform the method, step, simulation, algorithm, system, or system element.
Paragraph 1: In some embodiments, a method is provided. The method comprises contacting a probe with a fluid over a first period of time, wherein the fluid is supplied by a bioprocessing system, wherein the fluid is flowing over the probe, wherein an analyte is present in the fluid at a first concentration, and wherein at least a portion of the analyte becomes immobilized on the probe; detecting a variation of a signal over a first period of time; determining the first concentration based on the variation of the signal over the first period of time; and based on the determination of the first concentration, sending instructions to the bioprocessing system.
Paragraph 2: In some embodiments a system is provided. The system comprises a first instrument comprising a probe and a detector configured to detect a variation of a signal over a first period of time; and a bioprocessing system, wherein the system is configured to supply a fluid from the bioprocessing system to the first instrument, wherein the first instrument is configured to determine a first concentration of an analyte in the fluid while the fluid contacts and flows over the probe based on the variation of the signal over the first period of time, wherein the system is configured to send instructions to the bioprocessing system based on the determination of the first concentration.
Paragraph 3: In some embodiments, a method comprises contacting a probe with a fluid over a first period of time, wherein the fluid is flowing over the probe, wherein an analyte is present in the fluid at a first concentration, and wherein at least a portion of the analyte becomes immobilized on the probe; detecting a variation of an optical signal over the first period of time; and determining the first concentration based on the variation of the optical signal over the first period of time, wherein the optical signal comprises light reflected from an interface internal to the probe and light reflected from the end of the probe.
Paragraph 4: In some embodiments, a first instrument is provided. The first instrument comprises a probe; and an optical detector configured to detect a variation of an optical signal over a first period of time, wherein the first instrument is configured to determine a first concentration of an analyte in a fluid contacting and flowing over the probe based on the variation of the optical signal over the first period of time, and wherein the optical signal comprises light reflected from an interface internal to the probe and light reflected from the end of the probe.
Paragraph 5: In some embodiments, in a method, system, or first instrument as in any preceding paragraph, the fluid is as output by the bioprocessing system when contacted with the probe.
Paragraph 6: In some embodiments, in a method, system, or first instrument as in any preceding paragraph, the fluid is supplied to the first instrument as output from the bioprocessing system.
Paragraph 7: In some embodiments, in a method, system, or first instrument as in any preceding paragraph, the fluid is supplied to the first instrument in an automated manner.
Paragraph 8: In some embodiments, in a method, system, or first instrument as in any preceding paragraph, the instructions comprise modifying one or more properties of a fluid in the bioprocessing system.
Paragraph 9: In some embodiments, in a method, system, or first instrument as in any preceding paragraph, the system comprises a second bioprocessing system.
Paragraph 10: In some embodiments, in a method, system, or first instrument as in any preceding paragraph, the second bioprocessing system is configured to supply a second fluid output from the second bioprocessing system to the first instrument.
Paragraph 11: In some embodiments, in a method, system, or first instrument as in any preceding paragraph, the detector is configured to detect a variation of a second signal over a second period of time.
Paragraph 12: In some embodiments, in a method, system, or first instrument as in any preceding paragraph, the first instrument is configured to determine a second concentration of a second analyte in a second fluid while the second fluid contacts and flows over the probe based on the variation of the second signal over the second period of time.
Paragraph 13: In some embodiments, in a method, system, or first instrument as in any preceding paragraph, the system is configured to send second instructions to the bioprocessing system based on the determination of the second concentration.
Paragraph 14: In some embodiments, in a method, system, or first instrument as in any preceding paragraph, the second instructions comprise modifying one or more properties of a fluid in the bioprocessing system, continuing to supply the second fluid to the probe, supplying the second fluid to a different location, and/or taking no action.
Paragraph 15: In some embodiments, in a method, system, or first instrument as in any preceding paragraph, the bioprocessing system comprises a chromatography system.
Paragraph 16: In some embodiments, in a method, system, or first instrument as in any preceding paragraph, the system further comprises a second bioprocessing system, and wherein the second bioprocessing system comprises a bioreactor.
Paragraph 17: In some embodiments, in a method, system, or first instrument as in any preceding paragraph, the bioprocessing system comprises a bioreactor.
Paragraph 18: In some embodiments, in a method, system, or first instrument as in any preceding paragraph, the bioreactor is a batch-fed bioreactor.
Paragraph 19: In some embodiments, in a method, system, or first instrument as in any preceding paragraph, the bioreactor is a fed-batch bioreactor.
Paragraph 20: In some embodiments, in a method, system, or first instrument as in any preceding paragraph, the bioreactor is a perfusion bioreactor.
Paragraph 21: In some embodiments, in a method, system, or first instrument as in any preceding paragraph, the bioprocessing system comprises a filtration system.
Paragraph 22: In some embodiments, in a method, system, or first instrument as in any preceding paragraph, the filtration system is a tangential flow filtration system and/or an ultra/defiltration system.
Paragraph 23: In some embodiments, in a method, system, or first instrument as in any preceding paragraph, wherein the bioreactor comprises a centrifugation system and/or a centrifuge.
Paragraph 24: In some embodiments, in a method, system, or first instrument as in any preceding paragraph, the signal is an optical signal.
Paragraph 25: In some embodiments, in a method, system, or first instrument as in any preceding paragraph, the optical signal comprises light reflected from an interface internal to the probe and light reflected from the end of the probe.
Paragraph 26: In some embodiments, in a method, system, or first instrument as in any preceding paragraph, the optical signal comprises light reflected from a surface of the probe over a restricted angular range.
Paragraph 27: In some embodiments, in a method, system, or first instrument as in any preceding paragraph, the signal is a mechanical signal.
Paragraph 28: In some embodiments, in a method, system, or first instrument as in any preceding paragraph, the first instrument comprises a light source.
Paragraph 29: In some embodiments, in a method, system, or first instrument as in any preceding paragraph, the light source supplies light over a restricted wavelength range.
Paragraph 30: In some embodiments, in a method, system, or first instrument as in any preceding paragraph, the light source comprises an LED.
Paragraph 31: In some embodiments, in a method, system, or first instrument as in any preceding paragraph, the analyte is unlabeled.
Paragraph 32: In some embodiments, in a method, system, or first instrument as in any preceding paragraph, the fluid is supplied from a column in the chromatography system.
Paragraph 33: In some embodiments, in a method, system, or first instrument as in any preceding paragraph, the different location is a different column and/or chromatographic media in the chromatography system.
Paragraph 34: In some embodiments, in a method, system, or first instrument as in any preceding paragraph, the fluid is supplied from the probe to a waste receptacle.
Paragraph 35: In some embodiments, a method as in any preceding paragraph further comprises washing a column from which the fluid is supplied, and/or a system and/or first instrument as in any preceding paragraph is further configured to wash a column from which the fluid is supplied.
Paragraph 36: In some embodiments, a method as in any preceding paragraph further comprises eluting a column from which the fluid is supplied, and/or a system and/or a first instrument as in any preceding paragraph is further configured to elute a column from which the fluid is supplied.
Paragraph 37: In some embodiments, a method as in any preceding paragraph further comprises regenerating the column from which the fluid is supplied, and/or a system and/or a first instrument as in any preceding paragraph is further configured to regenerate the column from which the fluid is supplied.
Paragraph 38: In some embodiments, in a method, system, or first instrument as in any preceding paragraph, wherein the system comprises a processor in electronic communication with the detector, and wherein the processor is programmed to determine the first concentration based on the variation of the signal over the first period of time.
Paragraph 39: In some embodiments, a computer for implementing the method of any preceding paragraph is supplied, and/or a system and/or a first instrument of any preceding paragraph comprises a computer. The computer comprises an input interface configured to receive the signal, at least one processor programmed to determine the first concentration based on the variation of the signal over the first period of time, and an output interface configured to send instructions to the bioprocessing system.
Paragraph 40: In some embodiments, in a computer, system, or first instrument as in any preceding paragraph, the input interface is configured to receive the signal via at least one network.
Paragraph 41: In some embodiments, in a computer, system, or first instrument as in any preceding paragraph, the output interface is configured to indicate the first concentration of the analyte in the fluid.
Paragraph 42: In some embodiments, in a computer, system, or first instrument as in any preceding paragraph, the output interface comprises a display interface, and wherein indicating the first concentration of the analyte in the fluid comprises providing a numerical indication of the first concentration on the display interface.
Paragraph 43: In some embodiments, in a computer, system, or first instrument as in any preceding paragraph, the signal is received using an input interface, and wherein the first concentration is determined with the use of at least one processor.
Paragraph 44: In some embodiments, in a method, system, or first instrument as in any preceding paragraph, a computer-readable storage medium is encoded with a plurality of instructions that, when executed by a computer, perform the method of any preceding paragraph.
Paragraph 45: In some embodiments, in a method as in any preceding paragraph, the fluid contacted with the probe is a first sample supplied by a source of samples, and further comprising switching a valve positioned upstream of the probe to remove the source of samples from fluidic communication with the probe and place a source of a regeneration fluid in fluidic communication with the probe; and contacting the probe with the regeneration fluid. In some embodiments, a system and/or a first instrument of any preceding paragraph is configured to perform this method.
Paragraph 46: In some embodiments, in a method, system, or first instrument as in any preceding paragraph, the system and/or first instrument further comprises a valve positioned upstream of the probe; and a source of regeneration fluid positioned upstream of the valve, wherein: the valve is switchable between a plurality of positions, each position in the plurality of positions places the probe in fluidic communication with a source in a plurality of sources, the plurality of sources comprises a source of a regeneration fluid, and the plurality of sources comprises a source of samples.
Paragraph 47: In some embodiments, in a method as in any preceding paragraph, the fluid contacted with the probe is a first sample supplied by a source of samples, and the method further comprises closing a first valve to remove the source of samples from fluidic communication with the probe; opening a second valve to place a source of a regeneration fluid in fluidic communication with the probe; and contacting the probe with the regeneration fluid, wherein opening the first valve supplies the sample directly to the probe and/or opening the second valve supplies the regeneration fluid directly to the probe. In some embodiments, a system and/or first instrument of any preceding paragraph is configured to perform this method.
Paragraph 48: In some embodiments, in a method, system, or first instrument as in any preceding paragraph, a system and/or first instrument further comprises a plurality of valves positioned upstream of the probe, wherein: the plurality of valves comprises a first valve positioned valve positioned between a source of samples and the probe and a second valve positioned between a source of a regeneration fluid and the probe, and opening the first valve supplies the sample directly to the probe and/or opening the second valve supplies the regeneration fluid directly to the probe.
Paragraph 49: In some embodiments, in a method as in any preceding paragraph, the method is performed in a first instrument, the fluid is a first sample, the probe is a first probe, and the method further comprises, in the first instrument, performing the steps of: contacting the first probe with a first sequence of fluids, wherein the first sequence of fluids comprises the first sample and a regeneration fluid, contacting a second probe with a second sequence of fluids, wherein the second sequence of fluids comprises a second sample and the regeneration fluid, and subsequent to contacting the first probe with the regeneration fluid, contacting the first probe with a third sample. In some embodiments, a system and/or first instrument of any preceding paragraph is configured to perform this method.
Paragraph 50: In some embodiments, in a method, system, or first instrument as in any preceding paragraph, the system and/or first instrument further comprises a plurality of probes, wherein the plurality of probes comprises the probe; a plurality of inlets; and a source of a regeneration fluid, wherein: each inlet is in fluidic communication with a probe in the plurality of probes, each inlet is configured to be in reversible fluidic communication with the source of the regeneration fluid and/or a source of samples, the first instrument is configured to contact each probe alternately with the regeneration fluid and samples in a plurality of samples supplied from the source of samples.
Paragraph 51: In some embodiments, in a method, system, or first instrument as in any preceding paragraph, the signal or optical signal is associated with a sample.
Paragraph 52: In some embodiments, in a method as in any preceding paragraph, the method further comprises comparing the signal to a model signal profile. In some embodiments, a system and/or first instrument of any preceding paragraph is configured to perform this method.
Paragraph 53: In some embodiments, in a method, system, or first instrument as in any preceding paragraph, method as in claim 51, the model signal profile is associated with normal functioning of the first instrument.
Paragraph 54: In some embodiments, in a method, system, or first instrument as in any preceding paragraph, the model signal profile is associated with malfunctioning of the first instrument.
Paragraph 55: In some embodiments, in a method, system, or first instrument as in any preceding paragraph, the model signal profile is associated with the presence of bubbles in a fluid contacting the probe.
Paragraph 56: In some embodiments, in a method, system, or first instrument as in any preceding paragraph, the variation of the signal is its first derivative.
Paragraph 57: In some embodiments, in a method, system, or first instrument as in any preceding paragraph, the fluid is a first sample in a plurality of samples, and wherein an analyte is present in a first sample at a first concentration.
Paragraph 58: In some embodiments, in a method, system, or first instrument as in any preceding paragraph, the variation of the signal is indicative of an association constant between the analyte and a reagent immobilized on the probe.
Paragraph 59: In some embodiments, in a method, system, or first instrument as in any preceding paragraph, the variation of the signal is indicative of a dissociation constant between the analyte and a reagent immobilized on the probe.
Paragraph 60: In some embodiments, in a method, system, or first instrument as in any preceding paragraph, the variation of the signal is indicative of an affinity between the analyte and a reagent immobilized on the probe.
Paragraph 61: In some embodiments, in a method, system, or first instrument as in any preceding paragraph, the signal comprises fluorescent light.
Paragraph 62: In some embodiments, in a method, system, or first instrument as in any preceding paragraph, the regeneration fluid is configured to cause detachment of at least a portion of an analyte immobilized on the probe.
Paragraph 63: In some embodiments, in a method, system, or first instrument as in any preceding paragraph, the regeneration fluid comprises a buffer.
Paragraph 64: In some embodiments, in a method, system, or first instrument as in any preceding paragraph, the regeneration fluid buffer has a pH of greater than or equal to 1 and less than or equal to 5.
Paragraph 65: In some embodiments, in a method, system, or first instrument as in any preceding paragraph, the regeneration fluid buffer comprises glycine.
Paragraph 66: In some embodiments, in a method, system, or first instrument as in any preceding paragraph, the regeneration fluid buffer comprises sodium acetate.
Paragraph 67: In some embodiments, in a method, system, or first instrument as in any preceding paragraph, the regeneration fluid buffer comprises a citrate salt.
Paragraph 68: In some embodiments, in a method, system, or first instrument as in any preceding paragraph, the regeneration fluid buffer comprises a phosphate salt, tris buffer, and/or sodium hydroxide.
Paragraph 69: In some embodiments, in a method, system, or first instrument as in any preceding paragraph, the regeneration fluid comprises biotin.
Paragraph 70: In some embodiments, in a method, system, or first instrument as in any preceding paragraph, the regeneration fluid comprises histidin.
Paragraph 71: In some embodiments, in a method, system, or first instrument as in any preceding paragraph, the regeneration fluid comprises nickel ions.
Paragraph 72: In some embodiments, in a method, system, or first instrument as in any preceding paragraph, the first instrument comprises a source of a neutralization fluid.
Paragraph 73: In some embodiments, in a method, system, or first instrument as in any preceding paragraph, the neutralization fluid comprises a buffer.
Paragraph 74: In some embodiments, in a method, system, or first instrument as in any preceding paragraph, the neutralization fluid buffer has a pH of greater than or equal to 6 and less than or equal to 8.
Paragraph 75: In some embodiments, in a method, system, or first instrument as in any preceding paragraph, the neutralization fluid has a pH of greater than or equal to 9.
Paragraph 76: In some embodiments, in a method, system, or first instrument as in any preceding paragraph, the neutralization fluid buffer is phosphate-buffered saline.
Paragraph 77: In some embodiments, in a method, system, or first instrument as in any preceding paragraph, the neutralization fluid buffer is a tris buffer.
Paragraph 78: In some embodiments, in a method, system, or first instrument as in any preceding paragraph, the first instrument comprises a valve configured to switch between a source of the fluid, the source of a regeneration fluid, and a source of a neutralization fluid.
Paragraph 79: In some embodiments, in a method, system, or first instrument as in any preceding paragraph, the valve is positioned upstream of the inlet.
Paragraph 80: In some embodiments, in a method, system, or first instrument as in any preceding paragraph, the plurality of sources comprises a source of a neutralization fluid.
Paragraph 81: In some embodiments, in a method, system, or first instrument as in any preceding paragraph, the plurality of sources comprises a source of a fluid comprising a primary antibody.
Paragraph 82: In some embodiments, in a method, system, or first instrument as in any preceding paragraph, the plurality of sources comprises a source of a fluid comprising a secondary antibody.
Paragraph 83: In some embodiments, in a method, system, or first instrument as in any preceding paragraph, the plurality of sources comprises a source of a wash buffer.
Paragraph 84: In some embodiments, in a method, system, or first instrument as in any preceding paragraph, the plurality of sources comprises a source of fluid comprising a substrate for the secondary antibody.
Paragraph 85: In some embodiments, a method as in any preceding paragraph further comprises switching the valve to remove the source of the regeneration fluid from fluidic communication with the probe and place the source of the neutralization fluid in fluidic communication with the probe. In some embodiments, a system and/or first instrument as in any preceding paragraph is configured to perform this method.
Paragraph 86: In some embodiments, a method as in any preceding paragraph further comprises contacting the probe with the neutralization fluid. In some embodiments, a system and/or first instrument as in any preceding paragraph is configured to perform this method.
Paragraph 87: In some embodiments, a method as in any preceding paragraph further comprises switching the valve to remove the source of the neutralization from fluidic communication with the probe and place a source of samples in fluidic communication with the probe. In some embodiments, a system and/or first instrument as in any preceding paragraph is configured to perform this method.
Paragraph 88: In some embodiments, a method as in any preceding paragraph further comprises contacting a second sample supplied by the source of samples with the probe. In some embodiments, a system and/or first instrument as in any preceding paragraph is configured to perform this method.
Paragraph 89: In some embodiments, in a method, system, or first instrument as in any preceding paragraph, the first instrument comprises a waste receptacle.
Paragraph 90: In some embodiments, in a method, system, or first instrument as in any preceding paragraph, the waste receptacle is positioned downstream from an outlet.
Paragraph 91: In some embodiments, in a method, system, or first instrument as in any preceding paragraph, the first instrument further comprises a purification filter.
Paragraph 92: In some embodiments, in a method, system, or first instrument as in any preceding paragraph, the purification filter is positioned between a source of samples and the probe.
Paragraph 93: In some embodiments, in a method, system, or first instrument as in any preceding paragraph, the first instrument further comprises a degassing filter.
Paragraph 94: In some embodiments, in a method, system, or first instrument as in any preceding paragraph, the degassing filter is positioned between a source of samples and the probe.
Paragraph 95: In some embodiments, in a method, system, or first instrument as in any preceding paragraph, the first instrument further comprises a vacuum degasser.
Paragraph 96: In some embodiments, in a method, system, or first instrument as in any preceding paragraph, the first instrument further comprises an ultrasonic degasser.
Paragraph 97: In some embodiments, in a method, system, or first instrument as in any preceding paragraph, the first instrument further comprises a heater and/or cooler configured to perform degassing.
Paragraph 98: In some embodiments, in a method, system, or first instrument as in any preceding paragraph, the first instrument comprises a manifold.
Paragraph 99: In some embodiments, in a method, system, or first instrument as in any preceding paragraph, the manifold supplies fluids from the sources of fluids to the probe.
Paragraph 100: In some embodiments, in a method, system, or first instrument as in any preceding paragraph, the probe is in fluidic communication with a microfluidic channel positioned in the manifold.
Paragraph 101: In some embodiments, in a method, system, or first instrument as in any preceding paragraph, the microfluidic channel comprises a bend.
Paragraph 102: In some embodiments, in a method, system, or first instrument as in any preceding paragraph, the microfluidic channel comprises a step.
Paragraph 103: In some embodiments, in a method, system, or first instrument as in any preceding paragraph, the first instrument further comprises a temperature control system.
Paragraph 104: In some embodiments, in a method, system, or first instrument as in any preceding paragraph, the temperature control system is associated with the probe.
Paragraph 105: In some embodiments, in a method, system, or first instrument as in any preceding paragraph, the temperature control system is associated with tubing fluidically connecting the probe to a valve.
Paragraph 106: In some embodiments, in a method, system, or first instrument as in any preceding paragraph, the temperature control system is associated with a manifold.
Paragraph 107: In some embodiments, in a method, system, or first instrument as in any preceding paragraph, the first instrument further comprises a source of dilutant.
Paragraph 108: In some embodiments, in a method, system, or first instrument as in any preceding paragraph, the first instrument is configured to mix the dilutant with the plurality of samples upstream of the valve.
Paragraph 109: In some embodiments, a method as in any preceding paragraph further comprises contacting the probe with a plurality of fluids in a repeating cycle. In some embodiments, a system and/or first instrument as in any preceding paragraph is configured to perform this method.
Paragraph 110: In some embodiments, in a method, system, or first instrument as in any preceding paragraph, a second probe is contacted with a second sample while the probe is contacted with a regeneration fluid.
Paragraph 111: In some embodiments, in a method, system, or first instrument as in any preceding paragraph, a second probe is contacted with a second sample while a probe is contacted with the neutralization fluid.
Paragraph 112: In some embodiments, a method as in any preceding paragraph further comprises contacting two or more probes with a common sample at the same time. In some embodiments, a system and/or first instrument as in any preceding paragraph is configured to perform this method.
Paragraph 113: In some embodiments, in a method, system, or first instrument as in any preceding paragraph, the first instrument further comprises at least one probe that is not contacted with the sample while the two or more probes are contacted with the common sample.
Paragraph 114: In some embodiments, a method as in any preceding paragraph further comprises detecting a signal generated from each probe contacting the common sample. In some embodiments, a system and/or first instrument as in any preceding paragraph is configured to perform this method.
Paragraph 115: In some embodiments, in a method as in any preceding paragraph further comprises comparing the signals generated from the probes contacting the common sample. In some embodiments, a system and/or first instrument as in any preceding paragraph is configured to perform this method.
Paragraph 116: In some embodiments, a method as in any preceding paragraph further comprises determining whether there is an abnormality associated with one or more of the probes contacting the common sample based on the signal comparison. In some embodiments, a system and/or first instrument as in any preceding paragraph is configured to perform this method.
Paragraph 117: In some embodiments, in a method, system, or first instrument as in any preceding paragraph, the at least one probe is contacted with a fluid other than a sample.
Paragraph 118: In some embodiments, in a method, system, or first instrument as in any preceding paragraph, the plurality of fluids comprises a fresh sample of the fluid.
Paragraph 119: In some embodiments, in a method, system, or first instrument as in any preceding paragraph, the plurality of fluids comprises regeneration fluid.
Paragraph 120: In some embodiments, in a method, system, or first instrument as in any preceding paragraph, the plurality of fluids comprises a buffer.
Paragraph 121: In some embodiments, in a method, system, or first instrument as in any preceding paragraph, the plurality of probes comprises probes that differ from one another.
Paragraph 122: In some embodiments, in a method, system, or first instrument as in any preceding paragraph, the plurality of probes comprises two or more probes that do not differ from one another.
Paragraph 123: In some embodiments, in a method, system, or first instrument as in any preceding paragraph, none of the probes in the plurality of probes differ from one another.
Paragraph 124: In some embodiments, in a method, system, or first instrument as in any preceding paragraph, the plurality of probes comprises probes that are in series with each other.
Paragraph 125: In some embodiments, in a method, system, or first instrument as in any preceding paragraph, the plurality of probes comprises probes that are in parallel with each other.
Paragraph 126: In some embodiments, in a method, system, or first instrument as in any preceding paragraph, contacting the probe with a fluid comprises contacting the probe with the fluid for a period of time of greater than or equal to 1 second and less than or equal to 5 hours.
Paragraph 127: In some embodiments, in a method, system, or first instrument as in any preceding paragraph, contacting the probe with a fluid comprises contacting the probe with the fluid for a period of time of greater than or equal to 5 seconds and less than or equal to 1 minute.
Paragraph 128: In some embodiments, in a method, system, or first instrument as in any preceding paragraph, the repeating cycle occurs over a period of time of greater than or equal to 30 seconds and less than or equal to 90 seconds.
Paragraph 129: In some embodiments, in a method, system, or first instrument as in any preceding paragraph, the variation in the signal over time is indicative of a rate of binding of the analyte to the probe.
Paragraph 130: In some embodiments, in a method, system, or first instrument as in any preceding paragraph, the fluid is a crude sample.
Paragraph 131: In some embodiments, in a method, system, or first instrument as in any preceding paragraph, the crude sample further comprises a buffer.
Paragraph 132: In some embodiments, in a method, system, or first instrument as in any preceding paragraph, the crude sample further comprises one or more components of a cell media.
Paragraph 133: In some embodiments, in a method, system, or first instrument as in any preceding paragraph, the crude sample further comprises glucose.
Paragraph 134: In some embodiments, in a method, system, or first instrument as in any preceding paragraph, the crude sample further comprises lactate.
Paragraph 135: In some embodiments, in a method, system, or first instrument as in any preceding paragraph, the crude sample further comprises one or more types of amino acids.
Paragraph 136: In some embodiments, in a method, system, or first instrument as in any preceding paragraph, the crude sample further comprises one or more types of salt.
Paragraph 137: In some embodiments, in a method, system, or first instrument as in any preceding paragraph, the crude sample further comprises one or more types of protein.
Paragraph 138: In some embodiments, in a method, system, or first instrument as in any preceding paragraph, the one or more types of protein comprise protein A.
Paragraph 139: In some embodiments, in a method, system, or first instrument as in any preceding paragraph, the one or more types of protein comprise a host cell protein.
Paragraph 140: In some embodiments, in a method, system, or first instrument as in any preceding paragraph, the crude sample further comprises a peptide.
Paragraph 141: In some embodiments, in a method, system, or first instrument as in any preceding paragraph, the crude sample further comprises one or more types of nucleic acids.
Paragraph 142: In some embodiments, in a method, system, or first instrument as in any preceding paragraph, the crude sample further comprises one or more types of cells.
Paragraph 143: In some embodiments, in a method, system, or first instrument as in any preceding paragraph, the first instrument is in fluidic communication with an additional instrument.
Paragraph 144: In some embodiments, in a method, system, or first instrument as in any preceding paragraph, the first instrument is configured to divide a fluid flowing out of a second instrument into a plurality of samples.
Paragraph 145: In some embodiments, a method as in any preceding paragraph further comprises outputting a signal if the amount of an analyte is in excess of a pre-defined amount. In some embodiments, a system and/or first instrument as in any preceding paragraph is configured to perform this method.
Paragraph 146: In some embodiments, in a method, system, or first instrument as in any preceding paragraph, the signal is an electrical signal.
Paragraph 147: In some embodiments, in a method, system, or first instrument as in any preceding paragraph, the signal is transmitted via a standard specified in Open Platform Communications.
Paragraph 148: In some embodiments, in a method, system, or first instrument as in any preceding paragraph, the signal instructs an additional instrument to perform an action.
Paragraph 149: In some embodiments, in a method, system, or first instrument as in any preceding paragraph, the action is to halt.
Paragraph 150: In some embodiments, in a method, system, or first instrument as in any preceding paragraph, the action is to alter the flow of the fluid flowing out of an additional instrument.
Paragraph 151: In some embodiments, in a method, system, or first instrument as in any preceding paragraph, the action is to provide fluid flowing out of an additional instrument to a different receptacle.
Paragraph 152: In some embodiments, in a method, system, or first instrument as in any preceding paragraph, the action is to alter the flow of fluid flowing within an additional instrument.
Paragraph 153: In some embodiments, in a method, system, or first instrument as in any preceding paragraph, the fluid is received from an additional instrument in a manner preserves the sterility of the fluid remaining in the additional instrument.
Paragraph 154: In some embodiments, in a method, system, or first instrument as in any preceding paragraph, the additional instrument is a chromatography system.
Paragraph 155: In some embodiments, in a method, system, or first instrument as in any preceding paragraph, the additional instrument is a bioreactor.
Paragraph 156: In some embodiments, in a method, system, or first instrument as in any preceding paragraph, the additional instrument is a filtration device.
Paragraph 157: In some embodiments, in a method, system, or first instrument as in any preceding paragraph, the additional instrument is a centrifuge.
Paragraph 158: In some embodiments, in a method, system, or first instrument as in any preceding paragraph, the additional instrument is a pump.
Paragraph 159: In some embodiments, in a method, system, or first instrument as in any preceding paragraph, the additional instrument is a valve.
Paragraph 160: In some embodiments, in a method, system, or first instrument as in any preceding paragraph, a reagent is immobilized on the probe.
Paragraph 161: In some embodiments, in a method, system, or first instrument as in any preceding paragraph, the reagent is a protein.
Paragraph 162: In some embodiments, in a method, system, or first instrument as in any preceding paragraph, the protein is protein A.
Paragraph 163: In some embodiments, in a method, system, or first instrument as in any preceding paragraph, the protein is protein G.
Paragraph 164: In some embodiments, in a method, system, or first instrument as in any preceding paragraph, the protein is protein L.
Paragraph 165: In some embodiments, in a method, system, or first instrument as in any preceding paragraph, the reagent is a peptide.
Paragraph 166: In some embodiments, in a method, system, or first instrument as in any preceding paragraph, the reagent is an antibody.
Paragraph 167: In some embodiments, in a method, system, or first instrument as in any preceding paragraph, the reagent is an antigen.
Paragraph 168: In some embodiments, in a method, system, or first instrument as in any preceding paragraph, the reagent is a small molecule.
Paragraph 169: In some embodiments, in a method, system, or first instrument as in any preceding paragraph, the reagent is a virus.
Paragraph 170: In some embodiments, in a method, system, or first instrument as in any preceding paragraph, the reagent is a cell.
Paragraph 171: In some embodiments, in a method, system, or first instrument as in any preceding paragraph, the reagent is a differentiated cell type.
Paragraph 172: In some embodiments, in a method, system, or first instrument as in any preceding paragraph, the reagent is a polysaccharide.
Paragraph 173: In some embodiments, in a method, system, or first instrument as in any preceding paragraph, the reagent is a bacteria.
Paragraph 174: In some embodiments, in a method, system, or first instrument as in any preceding paragraph, the reagent is a nucleic acid.
Paragraph 175: In some embodiments, in a method, system, or first instrument as in any preceding paragraph, the nucleic acid is DNA.
Paragraph 176: In some embodiments, in a method, system, or first instrument as in any preceding paragraph, the reagent is streptavidin.
Paragraph 177: In some embodiments, in a method, system, or first instrument as in any preceding paragraph, the reagent is aminopropylsilane.
Paragraph 178: In some embodiments, in a method, system, or first instrument as in any preceding paragraph, the reagent is Ni-NTA.
Paragraph 179: In some embodiments, in a method, system, or first instrument as in any preceding paragraph, the reagent is lectin.
Paragraph 180: In some embodiments, in a method, system, or first instrument as in any preceding paragraph, the reagent is glutathione.
Paragraph 181: In some embodiments, in a method, system, or first instrument as in any preceding paragraph, the first instrument comprises an optical cable, and wherein the optical cable transmits light to the optical detector.
Paragraph 182: In some embodiments, in a method, system, or first instrument as in any preceding paragraph, the first instrument comprises a plurality of optical detectors.
Paragraph 183: In some embodiments, in a method, system, or first instrument as in any preceding paragraph, each optical detector in the plurality of optical detectors is associated with a probe.
Paragraph 184: In some embodiments, in a method, system, or first instrument as in any preceding paragraph, the probe is an optical probe.
Paragraph 185: In some embodiments, in a method, system, or first instrument as in any preceding paragraph, the probe is a fiber-optic probe.
Paragraph 186: In some embodiments, in a method, system, or first instrument as in any preceding paragraph, the first instrument comprises an optical cable.
Paragraph 187: In some embodiments, in a method, system, or first instrument as in any preceding paragraph, the probe transmits light through one or more apertures.
Paragraph 188: In some embodiments, in a method, system, or first instrument as in any preceding paragraph, one or more apertures are positioned on a side of the probe opposite an optical cable.
Paragraph 189: In some embodiments, in a method, system, or first instrument as in any preceding paragraph, the optical cable is configured to transmit light to the probe.
Paragraph 190: In some embodiments, in a method, system, or first instrument as in any preceding paragraph, the optical cable transmits light from a light source to the probe.
Paragraph 191: In some embodiments, in a method, system, or first instrument as in any preceding paragraph, the light source supplies light at a plurality of wavelengths.
Paragraph 192: In some embodiments, in a method, system, or first instrument as in any preceding paragraph, the light source is a halogen lamp.
Paragraph 193: In some embodiments, in a method, system, or first instrument as in any preceding paragraph, the first instrument comprises a plurality of light sources, each associated with a different probe.
Paragraph 194: In some embodiments, in a method, system, or first instrument as in any preceding paragraph, two or more probes are associated with a single light source.
Paragraph 195: In some embodiments, in a method, system, or first instrument as in any preceding paragraph, the first instrument comprises an optical switch configured to switch which probe a light source is associated with.
Paragraph 196: In some embodiments, in a method, system, or first instrument as in any preceding paragraph, an optical cable is configured to transmit light from the probe.
Paragraph 197: In some embodiments, in a method, system, or first instrument as in any preceding paragraph, the optical cable transmits light from the probe to an optical detector.
Paragraph 198: In some embodiments, in a method, system, or first instrument as in any preceding paragraph, the optical detector is a spectrometer.
Paragraph 199: In some embodiments, in a method, system, or first instrument as in any preceding paragraph, the analyte is a protein.
Paragraph 200: In some embodiments, in a method, system, or first instrument as in any preceding paragraph, the protein is protein A.
Paragraph 201: In some embodiments, in a method, system, or first instrument as in any preceding paragraph, the protein is a host cell protein.
Paragraph 202: In some embodiments, in a method, system, or first instrument as in any preceding paragraph, the protein is an Fc receptor.
Paragraph 203: In some embodiments, in a method, system, or first instrument as in any preceding paragraph, the analyte is a peptide.
Paragraph 204: In some embodiments, in a method, system, or first instrument as in any preceding paragraph, the analyte is an antibody.
Paragraph 205: In some embodiments, in a method, system, or first instrument as in any preceding paragraph, the antibody is IgG.
Paragraph 206: In some embodiments, in a method, system, or first instrument as in any preceding paragraph, the analyte is an antigen.
Paragraph 207: In some embodiments, in a method, system, or first instrument as in any preceding paragraph, the analyte is a small molecule.
Paragraph 208: In some embodiments, in a method, system, or first instrument as in any preceding paragraph, the analyte is a virus.
Paragraph 209: In some embodiments, in a method, system, or first instrument as in any preceding paragraph, the analyte is a capsid.
Paragraph 210: In some embodiments, in a method, system, or first instrument as in any preceding paragraph, the analyte is a cell.
Paragraph 211: In some embodiments, in a method, system, or first instrument as in any preceding paragraph, the analyte is a differentiated cell type.
Paragraph 212: In some embodiments, in a method, system, or first instrument as in any preceding paragraph, the analyte is a polysaccharide.
Paragraph 213: In some embodiments, in a method, system, or first instrument as in any preceding paragraph, the analyte is a bacteria.
Paragraph 214: In some embodiments, in a method, system, or first instrument as in any preceding paragraph, the analyte is a nucleic acid.
Paragraph 215: In some embodiments, in a method, system, or first instrument as in any preceding paragraph, the nucleic acid is DNA.
Paragraph 216: In some embodiments, in a method, system, or first instrument as in any preceding paragraph, the analyte is RNA.
Paragraph 217: In some embodiments, in a method, system, or first instrument as in any preceding paragraph, the analyte is mRNA.
Paragraph 218: In some embodiments, in a method, system, or first instrument as in any preceding paragraph, the analyte is an exosome.
Paragraph 219: In some embodiments, in a method, system, or first instrument as in any preceding paragraph, the analyte is an extracellular vesicle.
Paragraph 220: In some embodiments, in a method, system, or first instrument as in any preceding paragraph, the analyte is a plasmid.
Paragraph 221: In some embodiments, in a method, system, or first instrument as in any preceding paragraph, the analyte is an antibody fragment.
Paragraph 222: In some embodiments, in a method, system, or first instrument as in any preceding paragraph, the analyte is a nutrient component.
Paragraph 223: In some embodiments, in a method, system, or first instrument as in any preceding paragraph, the analyte is a metabolic.
Paragraph 224: In some embodiments, in a method, system, or first instrument as in any preceding paragraph, the analyte is a metabolic byproduct.
Paragraph 225: In some embodiments, in a method, system, or first instrument as in any preceding paragraph, the analyte is a hormone.
Paragraph 226: In some embodiments, in a method, system, or first instrument as in any preceding paragraph, the first instrument further comprises a controller.
Paragraph 227: In some embodiments, in a method, system, or first instrument as in any preceding paragraph, the controller provides instructions periodically.
Paragraph 228: In some embodiments, in a method, system, or first instrument as in any preceding paragraph, the controller provides instructions on demand.
Paragraph 229: In some embodiments, in a method, system, or first instrument as in any preceding paragraph, the controller provides instructions, and wherein the instructions are related to fluid flow.
Paragraph 230: In some embodiments, in a method, system, or first instrument as in any preceding paragraph, the controller provides instructions, and wherein the instructions are related to optical signal detection.
Paragraph 231: In some embodiments, in a method, system, or first instrument as in any preceding paragraph, the first instrument is interfaced with an additional instrument performing bioprocess.
Paragraph 232: In some embodiments, in a method, system, or first instrument as in any preceding paragraph, the first instrument monitors the bioprocess.
Paragraph 233: In some embodiments, in a method, system, or first instrument as in any preceding paragraph, the first instrument reports the results of a measurement performed on a fluid undergoing the bioprocess.
Paragraph 234: In some embodiments, in a method, system, or first instrument as in any preceding paragraph, the measurement is performed on a sample of the fluid undergoing the bioprocess.
Paragraph 235: In some embodiments, in a method, system, or first instrument as in any preceding paragraph, reporting of the measurement results has a time lag sufficiently low to enable bioprocess control.
Paragraph 236: In some embodiments, in a method, system, or first instrument as in any preceding paragraph, the analyte becomes immobilized on the probe as the fluid is flowing over the probe.
Paragraph 237: In some embodiments, in a method, system, or first instrument as in any preceding paragraph, the instructions comprise instructions to modify one or more properties of a fluid in the bioprocessing system, to supply the fluid to a different location, to pause, and/or to take no action.
Paragraph 238: In some embodiments, in a method, system, or first instrument as in any preceding paragraph, the fluid in the bioprocessing system differs from the fluid in one or more ways.
Paragraph 239: In some embodiments, in a method, system, or first instrument as in any preceding paragraph, the system comprises a processor in electronic communication with the detector, and wherein the processor is programmed to determine whether a threshold has been reached based on the variation of the signal over the first period of time.
Paragraph 240: In some embodiments, in a method, system, or first instrument as in any preceding paragraph, the output interface comprises a display interface, and wherein the display interface is configured to provide a numerical indication of a signal and/or a first derivative of a signal.
Paragraph 241: In some embodiments, a method as in any preceding paragraph further comprises outputting a signal if a variation of a derivative of an optical signal is in excess of a pre-defined amount. In some embodiments, a system and/or first instrument as in any preceding paragraph is configured to perform this method.

### Example 1

In this Example, advantages associated with detecting a variation in an optical signal over time are presented.

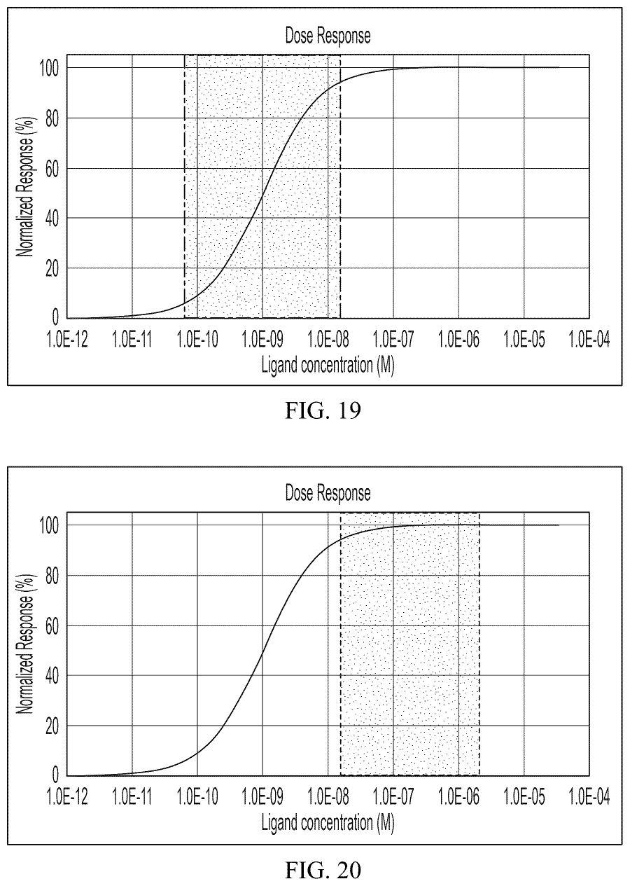

FIG. 19 shows simulated optical signals for equilibrium immobilization of an analyte on a probe for an analyte that has a binding constant to the probe of K_{D}=1 nM at various concentrations of the analyte in the fluid contacting the probe. As can be seen from FIG. 19, there is a range of analyte concentrations in the fluid that yield easily distinguishable optical signals (enclosed in the shaded rectangle). Analyte concentrations that are higher or lower than this value yield optical signals that are more challenging to resolve. This is shown schematically in FIG. 20, in which some such concentrations are enclosed in a shaded box.

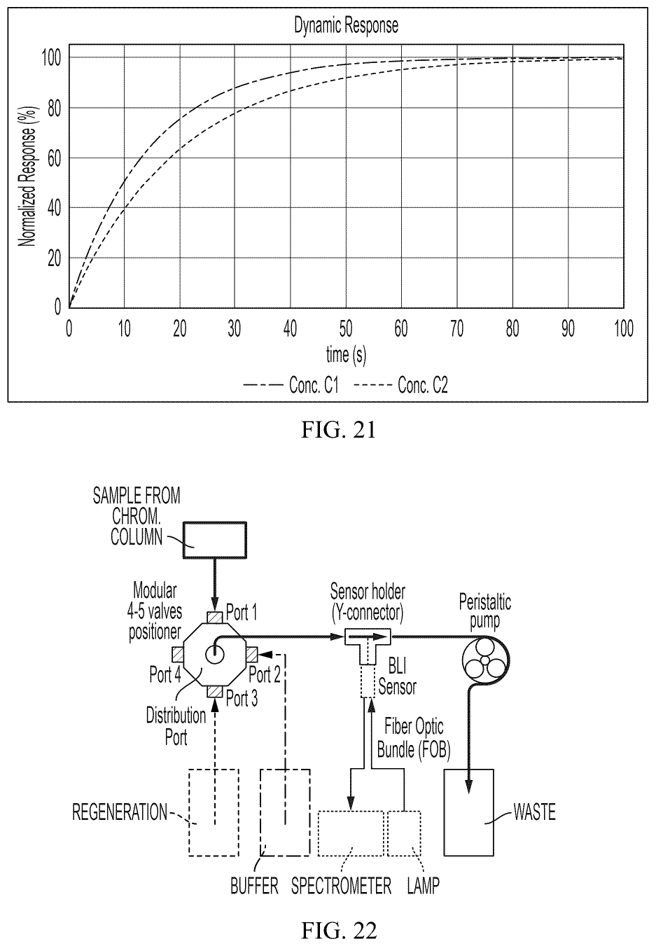

However, concentrations of analyte in the range shown in the shaded box may be easily resolved from each other if the variation of the optical signal over time is measured instead. This is shown schematically in FIG. 21. As can be seen in FIG. 21, fluids having different concentrations of an analyte that have very similar equilibrium levels of immobilization on the probe exhibit analyte immobilization on the probe at different rates. In FIG. 21, the concentration C1 is higher than the concentration C2, but fluids having both analyte concentrations display very similar equilibrium levels of analyte immobilization. However, the fluid having the analyte concentration C1 exhibits faster analyte immobilization than the fluid having the analyte concentration C2. Accordingly, measuring the variation of the optical signal over time may be employed to facilely determine the analyte concentration in such fluids with relatively high precision.

### Example 2

In this Example, a method for regenerating a probe is described.

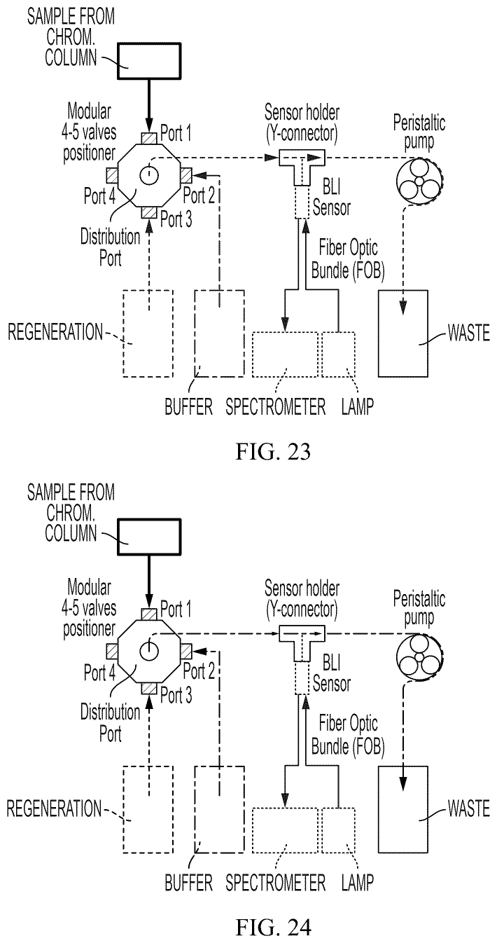

FIGS. 22-24 schematically depict a method in which a probe is contacted with a fluid possibly comprising an analyte and then regenerated. As shown in these FIGs., a three-way valve is positioned upstream of an inlet to a housing containing the probe. The three-way valve switches between positions that place a probe in fluidic communication with a source of a fluid (in this case, a chromatography column, labeled “SAMPLE FROM CHROM COLUMN”), a regeneration fluid, and a neutralization fluid (labeled “BUFFER”). A pump and a waste receptacle (labeled “WASTE”) are positioned downstream of the probe and the pump pumps a fluid from the housing containing the probe to the waste receptacle. As shown in FIG. 22, the first step is contacting a sample of a fluid from a chromatography column with a probe by flowing it across the probe. During this step, a variation in an optical signal is detected and employed to determine a concentration of the analyte in the sample of the fluid. Then, as shown in FIG. 23, the three-way valve selects the regeneration fluid for contact with the probe and supplies the regeneration fluid, which then flows across the probe and then into the waste receptacle. Finally, as shown in FIG. 24, the three-way valve selects the neutralization fluid for contact with the probe and supplies the neutralization fluid, which then flows across the probe and into the waste receptacle. After the step shown in FIG. 24, the step shown in FIG. 22 (and possibly the steps shown in FIGS. 23 and 24) may be repeated.

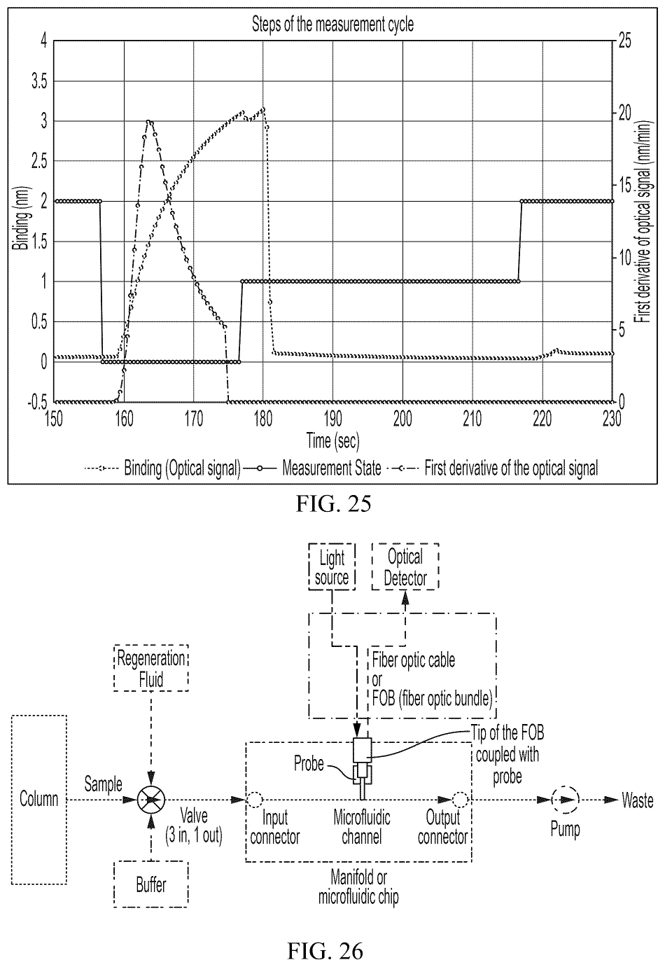

FIG. 25 shows the amount of binding of the analyte to the probe during each of the steps described above when employed in a system for which the chromatography column supplied a fluid comprising an analyte that is a monoclonal antibody and for which the probe is functionalized with protein A. As can be seen in FIG. 25, during contact of the probe with the fluid comprising the analyte (method step no. 0), both the optical signal and its first derivative (as smoothed by a numerical filter) increase, the latter increasing more rapidly than the former. Then, upon contact of the probe with the regeneration fluid (method step no. 1), the optical signal drops to a value close to zero and its first derivative becomes zero. This is believed to be due to the decrease in the pH of the fluid contacted with the probe during regeneration, which is believed to cause the affinity of the monoclonal antibody for protein A to decrease and thereby cause the monoclonal antibody to detach from the probe. The optical signal and its first derivative remain relatively constant upon the subsequent exposure of the probe to the neutralization fluid (method step no. 2). The increase in the pH of the fluid contacted with the probe during neutralization was believed to allow for protein A binding during subsequent contact of the probe with a sample of the fluid.

### Example 3

The method described in Example 2 may also be performed in an instrument comprising one or more microfluidic channels instead of tubing. FIG. 26 depicts one non-limiting example of such an instrument. As shown in FIG. 26, an instrument may comprise a housing comprising a microfluidic channel, a probe in fluidic communication with the microfluidic channel, and a switchable valve positioned upstream of the probe. The switchable valve positioned upstream of the probe may comprise three positions: a position that places the probe in fluidic communication with the column, a position that places the probe in fluidic communication with a source of a buffer, and a position that places the probe in fluidic communication with a source of a regeneration fluid. The switchable valve may be switched between these three positions in order to place the probe in fluidic communication with these three sources of fluid (possibly, in the order of the column, then the source of the regeneration fluid, and then the source of the buffer). The valve may be switched between these three positions in a repeating manner in order to alternately contact the probe with a sample of a fluid supplied by the column and regenerate the probe. During contact of the probe with the samples of the fluid, the amount of an analyte in the samples of the fluid may be detected. This process may be repeated or a preset period of time, until the analyte exceeds a preset amount, and/or until an operator halts operation.

### Example 4

In this Example, an instrument comprising a plurality of probes is described.

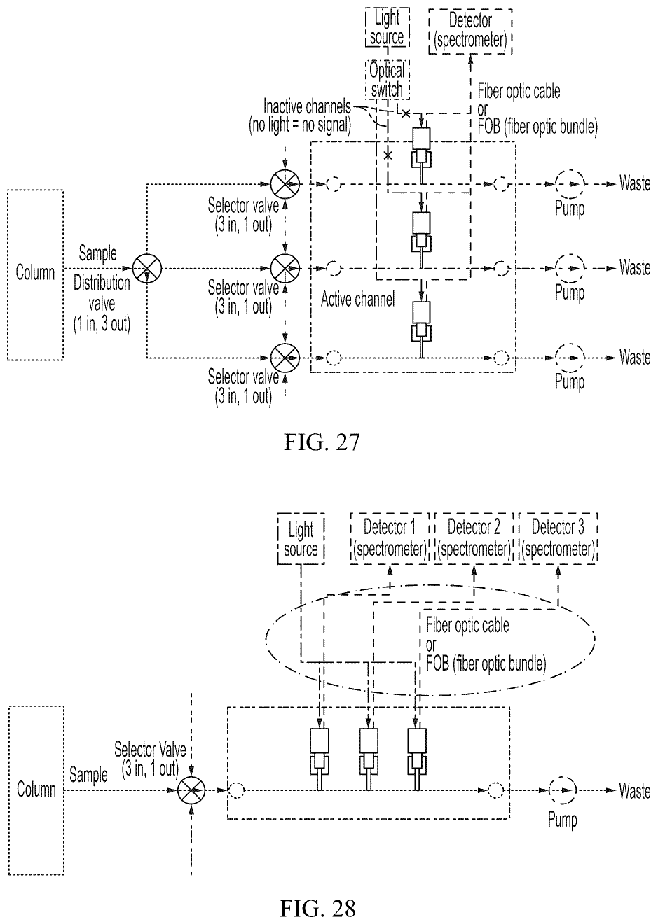

An instrument similar to the instrument comprising a microfluidic channel described in Example 2 may be provided, except that it may comprise a plurality of valves and a plurality of probes. Such an instrument is shown schematically in FIG. 27. The plurality of valves may comprise an upstream-most switchable valve that comprises a plurality of positions, each of which places the column in fluidic communication with a downstream switchable selector valve in a plurality of downstream switchable selector valves.

The upstream-most switchable valve may be switched between different positions in order to place different downstream selector valves in fluidic communication with the column. Each downstream switchable selector valve may be associated with a probe in the plurality of probes, and may comprise three positions: a position that places the probe with which it is associated in fluidic communication with the upstream-most switchable valve, a position that places the probe in fluidic communication with a source of a buffer, and a position that places the probe in fluidic communication with a source of a regeneration fluid.

The downstream switchable selector valves and upstream-most switchable valve may be switched together in order to place each probe in fluidic communication with these three sources of fluid (possibly, in the order of the column, then the source of the regeneration fluid, and then the source of the buffer). This switching may also cause samples of the fluid from the column to be continuously provided to the instrument and for at least one probe in the plurality of probes to be in fluidic communication with the column at all times.

### Example 5

In this Example, an instrument comprising a plurality of different types of probes is described.

FIG. 28 shows one non-limiting example of an instrument comprising a plurality of different types of probes. The instrument shown in FIG. 28 is similar to that described in Example 2 comprising a microfluidic channel except that it comprises three different types of probes. The three different types of probes are arranged serially downstream of the valve. Fluid flowing through the instrument may flows over each of these types of probes serially. Such instruments may be useful when, for example, a sample of a fluid comprises more than one analyte whose concentration it would be desirable to detect. The different types of probes may be configured to detect different types of analytes, and so may allow for the detection of multiple types of analytes in a single instrument and/or a single flowing fluid.

### Example 6

In this Example, an instrument comprising two filters, a source of a dilutant, and a temperature control system is described.

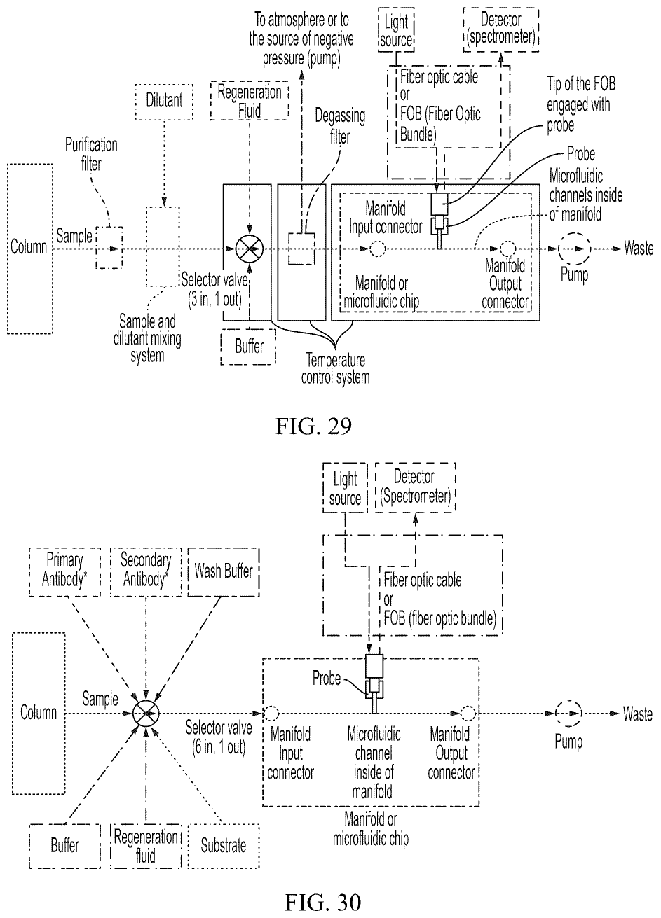

FIG. 29 shows one non-limiting example of an instrument comprising a purification filter, a degassing filter, a source of a dilutant, and a temperature control system. The instrument shown in FIG. 29 is similar to that described in Example 2 comprising a microfluidic channel except that it includes these additional components. The purification filter may be placed upstream of the source of the dilutant, and the degassing filter may be placed downstream of the switchable valve. The temperature control system may be suitable for controlling the temperature of the switchable valve, the degassing filter, and/or the housing.

### Example 7

In this Example, an instrument comprising a switchable valve comprising seven positions is described.

FIG. 30 shows one non-limiting example of an instrument comprising a switchable valve comprising seven positions. The instrument shown in FIG. 30 is similar to that described in Example 2 comprising a microfluidic channel except that the switchable valve has more positions. The switchable valve shown in FIG. 30 can reversibly place the probe in fluidic communication with a column, a source of a primary antibody, a source of a secondary antibody, a wash buffer, a regeneration fluid, and a substrate for the secondary antibody. Instruments like those shown in FIG. 30 may be suitable for performing assays on samples of the fluid obtained from the column.

### Example 8

In this Example, a method of detecting breakthrough of an analyte from a chromatography column is described.

A monoclonal antibody was produced in a device employed to perform continuous perfusion cultivation of a Chinese hamster ovarian cell line. Alternating tangential flow filtration was employed to remove the cells from the perfusion permeate, which was collected in a surge vessel. The surge vessel served as a feed solution for affinity chromatography performed in a continuous simulated moving bed chromatography system. The continuous simulated moving bed chromatography system was in fluidic communication with an instrument comprising a probe functionalized with protein A as a reagent. The instrument was operated similarly to the instrument described in Example 2. Here, the continuous simulated moving bed chromatography system comprising built-in chromatography columns served as the chromatography column, the regeneration fluid was a 10 mM glycine buffer having a pH of 2, and the neutralization fluid was phosphate buffered saline having a pH of 7.4. The cycle over which these three fluids were contacted with the probe occurred over one minute.

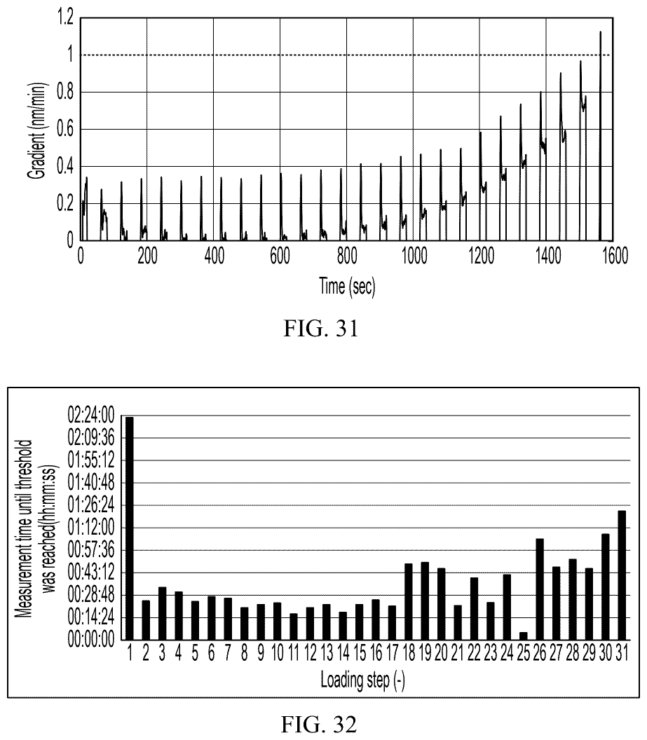

FIG. 31 shows the first derivative of the optical signal measured over a period of 27 cycles (labeled therein as “gradient”). For the first 800 seconds, the optical signal had a constant and low first derivative of below 0.4 nm/min. This supports the proposition that no significant breakthrough of the monoclonal antibody occurred during this period of time. Then, the first derivative of the optical signal began to increase, which was indicative of monoclonal antibody breakthrough. Finally, the first derivative of the optical signal increased above a previously defined threshold value of 1 nm/min (shown in FIG. 31 as a dotted line), which triggered a column switch in which the fluid flowing out of the column in the continuous simulated moving bed chromatography system and contacted with the probe was diverted to instead flow to another column therein. At this point in time, the feed solution from the surge tank was diverted to another column in accordance with a simulated moving bed chromatography process.

Offline measurements were performed on the diverted flow and indicated that the concentration of the monoclonal antibody in the fluid was 0.02 g/L at the time that the threshold value was reached, which was indicative of an insignificantly low loss of the monoclonal antibody. After the column switch was triggered, the flow from the other column was not supplied to the probe for 108 minutes, a period of time shorter than the shortest possible breakthrough time for the other column. Subsequently, flow from the other column was supplied to the probe, the optical signal associated with the probe was measured, the instrument was again operated as described above, and the time until column breakthrough was recorded.

The process described in the preceding paragraph was repeated for a period of time of three days, and the amount of measurement time to column breakthrough for each operation of the instrument is shown in FIG. 32. The overall yield for the monoclonal antibody (i.e., 100% multiplied by the ratio of the monoclonal antibody in the final eluate to all of the monoclonal antibody recovered from the continuous simulated moving bed chromatography system) was 96.9% and the monoclonal antibody concentration in the final eluate was 15.1 g/L (the approximate concentration of the monoclonal antibody in the feed solution was 0.7 g/L). Additionally, DNA and HCP impurities were removed to a high degree (i.e., 2.1 log removal to 30 ppm and 3.6 log removal to 38 ppm, respectively).

### Example 9

### Abstract

Biopharmaceuticals, like monoclonal antibodies (mAbs), are used for treatment of numerous severe diseases like cancer, infections autoimmune disorders and inflammatory diseases. Due to the high specificity, activity and fewer side effects compared to conventional drugs, the market for mAbs rises continuously. In order to improve manufacturing economics, flexibility and to obtain a more consistent product quality continuous or semi-continuous biomanufacturing for process intensification would be desirable. However, due to the high process complexity, the difficulties in (digital) process integration and the reaction to process variations, there are still numerous challenges to be overcome.

In this study, a fully integrated and continuous upstream and capture step with an overarching control strategy for the production of a mAb is shown. Advanced control strategies were implemented by interfacing the involved equipment as well as novel process analytical technologies with an overarching custom software component. This custom software component was used to trigger and control specific equipment actions based on monitored process data. Starting from a perfusion process with an alternating tangential flow module for cell retention, the mAb was captured from the permeate by a simulating moving bed (SMB) affinity chromatography. Setpoint changes and variations throughout the upstream process were addressed by a dynamic flow control. In addition, dynamic loading of the SMB was realized by a new developed sensor, based on continuous biolayer interferometry measurements, to detect mAb breakthrough within the flow-through. This novel approach offers several benefits such as a high specificity and low background signal, resulting in desirable (e.g., high) resin utilization while simultaneously reducing (and, in some instances, avoiding) product loss.

A robust continuous process was operated for several days, assisted by the overarching control strategy, with a high yield and purity of the mAb. The results of this study and the novel employed analytical approaches offer great potential for the establishment of adaptive continuous manufacturing of biopharmaceuticals.

### INTRODUCTION

Monoclonal antibodies (mAbs) are used for treatment of numerous severe diseases like cancer, infections autoimmune disorders and inflammatory diseases. Due to the high specificity, activity and fewer side effects compared to conventional drugs the market for mAbs rises continuously, representing over 60% of the global biopharmaceutical industry revenue.

In order to reduce manufacturing costs, which would allow broader patient access, continuous or semi-continuous biomanufacturing for process intensification would be desirable. For continuous upstream process (USP) cultivations, improvements would be desirable. Some USP cultivations undesirably require the continuous addition of unspent media, removal of the spent media containing the mAb by permeate withdrawal and the retaining of the cells in the bioreactor. Accordingly, USP implementation in the highly regulated biomanufacturing industry remains challenging due to the complexity of the process development and the required automated control strategy. Therefore, there is not yet a wide use in the industry for the production of mAb.

To avoid a bottleneck and fully exploit the possible benefits of such a continuous USP, an intensified downstream process (DSP) would be beneficial. Some DSP processes undesirably exhibit limited loading capacity and high cost. Mitigating such disadvantages by cycling several times can negatively result in reduced throughput, longer processing times and related potential impacts on the product quality.

The application of continuous simulated moving bed (SMB) chromatography as approach for DSP intensification offers an increased productivity with simultaneous reduced buffer consumption and manufacturing facility footprint, while maintaining product yield and purity at laboratory as well as pilot scales. The implementation of SMB technology allows the resin particles to remain in a packed bed while the “transport” of the resin is realized by means of switching column in- and outlets. By the usage of a multi column chromatography (MCC) approach product breakthrough of one column is loaded on another one, avoiding product loss while simultaneously improving resin utilization, exceeding the dynamic binding capacity, can be obtained. This allows for significant improvements, as large batch chromatography columns can sometimes be loaded to only 65% of their static binding capacity. Once fully loaded a column switch is applied and the column is washed, eluted and regenerated, before entering the next loading cycle. Using a countercurrent sequence, the efficiency of the process further improves due to enhancing the driving force for mass transfer throughout the entire course of the contact zone. As a result, the mass transfer can exceed the thermodynamic limitations of a batch or concurrent chromatography process.

It can be undesirable for continuous SMB processes to rely on predefined recipes with regard to loading volumes and flow rates and consequently for the column switch time. This requires several assumptions like a constant and known product concentration in the feed solution, that all columns have the same binding capacity and that the binding capacity remains constant throughout the process. Any deviations from these assumptions result in either inefficient processes with a low productivity or a low yield due to product loss. A dynamic loading could be obtained by a breakthrough detection of the mAb within the flow-through of the last column in the loading cycle, which then triggers the column switch. This offers several benefits like the ability to compensate for variable mAb concentrations in the feed solutions (e.g. by variations in the cell cultivation) and a reduction of product loss due to variations in the column capacity.

To perform (near) real-time process monitoring and control, several process analytical technology (PAT) tools can be used for fast mAb quantification, like IR or Raman spectroscopy. UV spectroscopy is an example of another technique that can be used for mAb breakthrough detection. One approach is the calculation of a difference signal of two detectors, situated at the column inlet and the column outlet using the UV absorption at 280 nm. Another approach is to employ partial least squares regression modeling on UV/Vis absorption spectra instead of single wavelength measurements. However, these methods offer several disadvantages like the low specificity of the UV signal (mAb and impurities contribute to the absorption at 280 nm) and the very high background signal due to the media components in the feed solution. This poses challenges to detect a very low mAb concentration within the flow-through in order to avoid product loss.

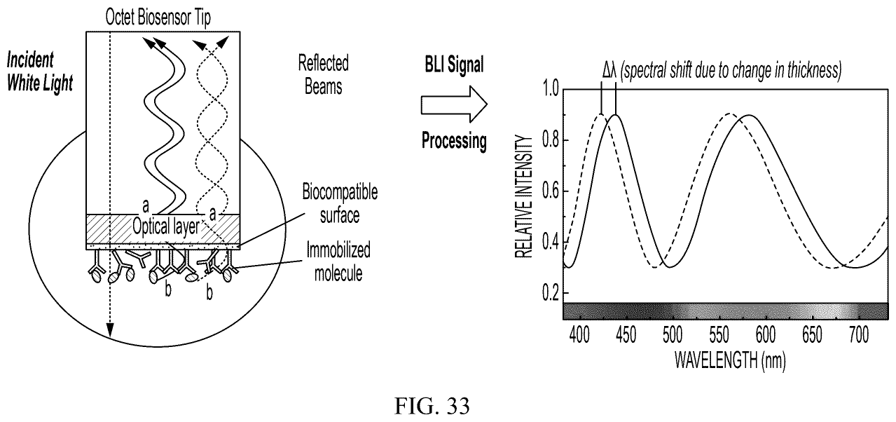

A promising method to overcome these limitations might be Bio-layer Interferometry (BLI). This label-free technology is based on the interference pattern obtained on combination of white light reflected from an internal reference surface and a bio-layer (FIG. 33). The high affinity and consequently binding of the analyte to the immobilized molecule at the biocompatible surface result in an increased thickness at the surface of the biosensor, which in turn is measured as concentration dependent signal by the change in the light interference (FIG. 33). For the quantification of mAbs, specific biosensors with immobilized protein A on their surface can be used.

The application for process control strategies, like the dynamic loading of a continuous SMB chromatography, would be benefitted by on- or at-line measurements.

In this study, a novel BLI-based prototype is presented, allowing for continuous at-line measurements of mAbs for breakthrough detection of a continuous SMB chromatography process. This approach was implemented in an overarching control strategy for a dynamic loading as well as dynamic flow control. A continuous mAb perfusion cultivation was combined with a continuous capture step and executed for over 3 days controlled completely autonomously by the established overarching control strategy.

Further, Surface plasmon resonance (SPR) technology may be used to perform one or more process control strategies disclosed herein, such as the dynamic loading and dynamic flow control of continuous SMB chromatography. Like BLI, SPR is a label-free binding technique that can measure binding kinetics of biomolecular interactions in real time. In SPR, a sensor chip is used to immobilize one or more binding partners onto its surface. SPR technology may enable continuous on- or at-line measurements of mAbs for breakthrough detection of a continuous SMB chromatography process to help avoid product loss.

SPR technology also offers benefits for mAb breakthrough detection. As with BLI, one of the benefits afforded by SPR technology is increased specificity, which results in lower background signal, which allows lower limits of detection for earlier detection of mAb breakthrough. This is particularly important for detecting a very low mAb concentration in order to avoid product loss.

### Material and Methods

### Perfusion Cultivation

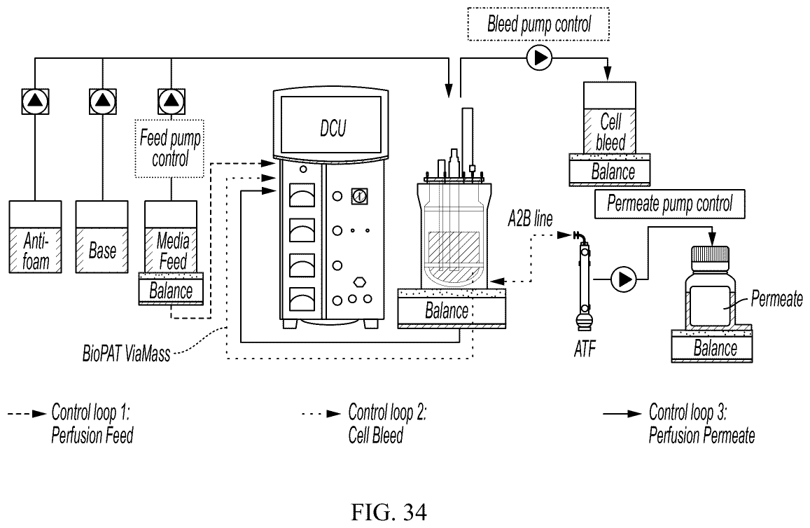

A 2 L Univessel® Glass bioreactor (Sartorius, Germany) was inoculated at 0.2.106 cells/mL from standard batch seed cultures of a Cellca CHO DG44 cell line (Sartorius, Germany) expressing a mAb of the type IgG1. The process was controlled at 36.8° C., pH 7.1 and DO 60%. After a three-day batch phase, pH was shifted to 6.95 and perfusion was started at 1 vessel volume per day (VVD) using a proprietary perfusion media formulation. An ATF2 device (Repligen, USA) was connected to the bioreactor bottom drain for cell retention. During the initial cell growth phase, the perfusion rate was increased as required to maintain a cell specific perfusion rate (CSPR) of 50 pL/cell/day. The process control strategy is illustrated in FIG. 34. In summary, perfusion rate was controlled using a gravimetric feed flow controller in combination with removal of cell-free permeate to maintain a constant working volume of 2 L. An automatic cell bleed was utilized to maintain the target viable cell concentration (VCC). This was achieved using a PID controller in BioPAT® MFCS (Sartorius, Germany) to control the bleed pump speed based on in-line biomass measurement (BioPAT® ViaMass, Sartorius, Germany).

### Continuous Capture

The cell free perfusion permeate was transferred to a 2 L intermediate surge tank and from there continuous processed by a BioSMB PD chromatography system (Sartorius, Germany). 5 mL MabSelect SuRe™ pre-packed HiTrap columns (2.5 cm column height, 1.6 cm column diameter, Cytiva, USA) were used for the affinity mAb capture. The initial recipe for all phases was: loading of the perfusion permeate, 3 column volumes (CV) interconnected wash, 8.5 CV wash, 4 CV elution, 5 column CV equilibration, 5 CV cleaning in place (CIP) and 5 CV equilibration. All used buffer chemicals for equilibration and wash (PBS, pH 7.4), elution (50 mM C₂H₃NaO₂, pH 3.0) as well as CIP (0.1 M NaOH) were purchased from Carl Roth (Germany). The flow rate was kept constant at 1.3 mL/min for all steps except the loading.

For the advanced control strategies the dynamic flow control as well as dynamic loading function of the BioSMB PD system were used with the respective OPC UA interface. Three phases with different flow rates (low=2.1 mL/min, medium=2.4 mL/min and high=2.7 mL/min) of the loading but based on the same recipe with equal number of transitions and relative duration of each step were used for the dynamic flow rate function to allow a change between phases during operation. Based on the dynamic loading function, loading of perfusion permeate was continued over the minimum loading volume of 52 CV until mAb was detected in the flow-through of the last column in the loading cycle.

### Continuous Biolayer Interferometry

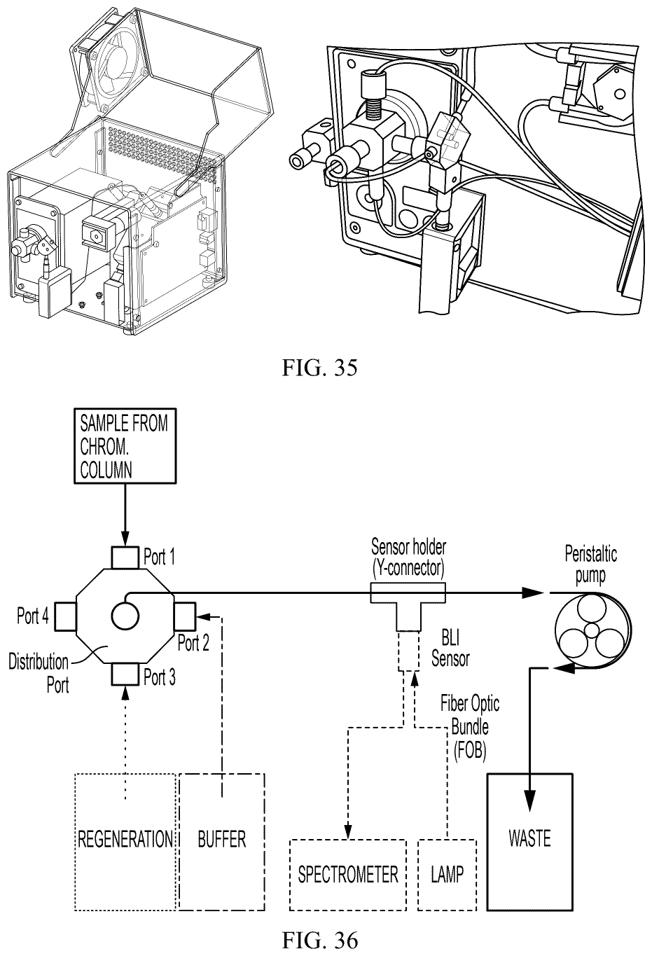

The mAb breakthrough in the flow-through of the SMB capture process was measured by a continuous BLI prototype (FIG. 35). For a specific mAb measurement a biosensor with immobilized protein A was used (FIGS. 35-36, Octet ProA biosensor, Sartorius, Germany).

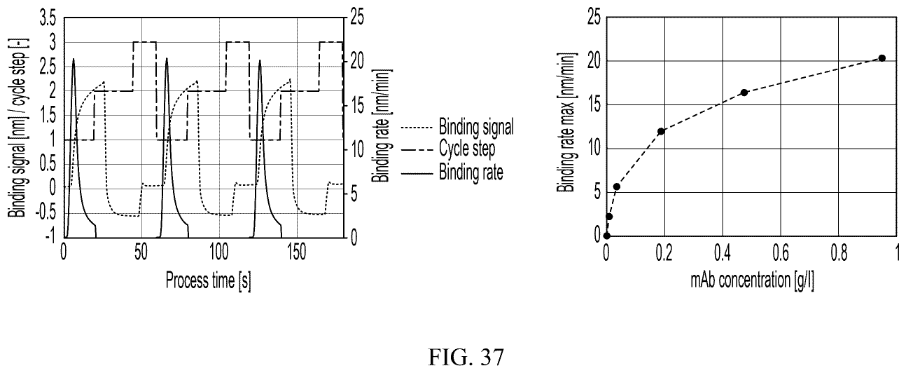

One measurement cycle consisted of the following steps: sample application (step number 1; flow-through of the last sequential connected column during the loading step), regeneration (step number 2; 10 mM glycine, adjusted with HCl to pH 2.0) and neutralization (step number 3; PBS, pH 7.4). A complete cycle lasted 60 seconds (FIG. 37 left). The different solutions required for each of the steps were provided by the use of a valve upstream and a pump downstream of the biosensor (FIG. 35 left, FIG. 36). For all steps the pump rate was 120 rpm resulting in a flow rate of 1.09 mL/min. The biosensor was illuminated by a lamp with white light and the interference of the reflected light from an internal coating within the biosensor and the end of the probe was detected by a spectrometer (FIG. 36). According to the principle of BLI immobilization of the mAb to the protein A biosensor resulted in an increase of the thickness at the end of the probe resulting in an increased wavelength shift and binding signal (FIG. 37 left). This in turn is proportional to the mAb concentration in the solution.

In order to obtain a concentration dependent signal with low lag time the binding rate of step number 1, corresponding to the first derivative of the binding signal was used. In FIG. 37 left an exemplary course of the binding signal and the binding rate is shown for the three steps for three consecutive cycles. From this it becomes apparent, that the maximum binding rate value is obtained more rapidly compared to the binding signal itself which reaches a maximum plateau only after a prolonged period of time (FIG. 37 left). The maximum obtained binding rate as a function of the mAb concentration for purified mAb samples diluted in PBS revealed an explicit, non-linear correlation (FIG. 37 right).

### Control Strategy Architecture

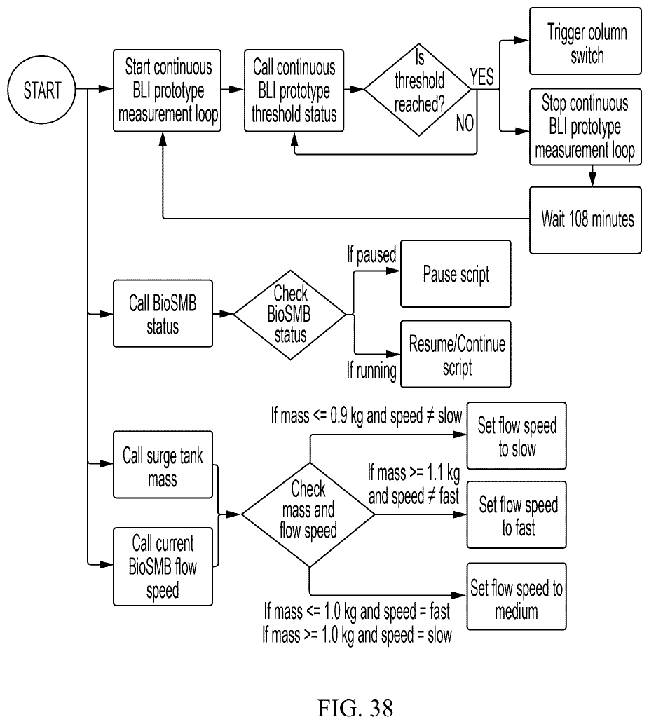

The process control component used to orchestrate the instruments embedded into the continuous bioprocess was implemented using the Node-RED integrated development environment (Version 1.3.4, OpenJS Foundation, CA, USA). All devices were part of the same network infrastructure. The communication nodes to interact with the laboratory equipment were “node-red-contrib-opcua” for the interaction with the OPC UA interfaces of the BioSMB and the permeate surge tank balance (Cubis II, Sartorius, Germany) implemented into BioPAT® MFCS, as well as the inbuilt TCP node for the interaction with the continuous BLI prototype (all Sartorius Stedim Biotech GmbH, Göttingen, Germany). Timer related events were implemented using the “node-red-contrib-looptimer-advanced” node. Furthermore, the inbuilt core nodes were used to establish the control script (FIG. 38). The software component was deployed on a Raspberry Pi 4 B 4 GB single-board computer running on Raspberry Pi OS (Debian 11; Kernel 5.15, Raspberry Pi Foundation, United Kingdom).

### Analytics

For the determination of mAb concentration size exclusion chromatography (SEC) was used as high performance liquid chromatography (HPLC) with a Dionex UltiMate 3000 HPLC System (ThermoFisher Scientific, Waltham, USA) and a Yarra 3 μm SEC 3000 column (Phenomenex, Torrance, USA) at a flow rate of 1 mL/min. A SEC buffer solution containing 0.1 M Na₂SO₄, 0.05 M NaH₂PO₄ and 0.05 M Na₂HPO₄ (all chemicals purchased from Carl Roth, Karlsruhe, Germany) with a final pH of 6.6 was used. The SEC method was previously verified with analytical protein A HPLC to ensure appropriate mAb determination (data not shown). If necessary, samples were diluted with SEC buffer prior to analysis and filtered through a Minisart RC4 0.2 μm syringe filter (Sartorius, Germany, Göttingen). The mAb concentration was determined based on the peak area (at 220 nm) of a calibration curve obtained using a known reference mAb material.

The HCP concentration was determined by a CHO HCP-ELISA (Cygnus Technologies, Southport, USA) according to the manufacturer's instructions. Samples were diluted, if necessary, in ELISA buffer (20 mM TRIS, 50 mM NaCl; all chemicals purchased from Karl Roth, Karlsruhe, Germany). Measurement was performed with an Infinite M Nano Plus plate reader (Tecan Trading AG, Switzerland) with a limit of detection of 1 ng/mL.

Concentration of DNA was measured by the Quant-iT™ PicoGreen™ dsDNA Assay Kit (ThermoFisher Scientific, Waltham, USA). Samples were diluted in TE buffer (10 mM TRIS, 1 mM EDTA, 0.1% SDS; all chemicals purchased from Karl Roth, Karlsruhe, Germany) if necessary. The measurement was performed by an Infinite M Nano Plus plate reader with a limit of detection of 31.25 ng/mL.

The parts per million (ppm) value of HCP and DNA (ppmᵢₘₚ) was calculated according to Eq. 1, where cᵢₘₚ is the concentration of the respective impurity and c_{mAb} is the concentration of the mAb.

ppmᵢₘₚ=(cᵢₘₚ/c_{mAb})×1 000 000 (Eq. 1)

### Results and Discussion

### Continuous USP Perfusion Cultivation

To allow for the continuous production of a mAb, a 2 L stirred tank bioreactor was operated in perfusion mode. By utilizing a membrane-based cell retention device, the product could be continuously withdrawn from the bioreactor and transferred to an intermediate surge tank before further processing using the continuous chromatography system. FIG. 34 shows a schematic overview of the perfusion setup and the main control loops which were implemented to enable a fully automated and robust process even at high cell concentrations. A gravimetric flow controller was used to maintain a steady addition of fresh perfusion media. The permeate controller was to be coupled to the bioreactor weight to maintain a constant working volume. As the working volume is not only affected by the addition of feed but also current bleeding rate as well as addition of corrective agents, e.g. for foam or pH control, this resulted in the permeate flow rate being more unpredictable and susceptible to fluctuations. In order to compensate for the resulting fluctuations in permeate flow rate and to enable a robust, integrated process, a dynamic flow rate regulation was implemented as part of the overarching DSP control strategy. Due to the obtained reliable cultivation conditions and optimal supply of nutrients this control strategy was highly advantageous.

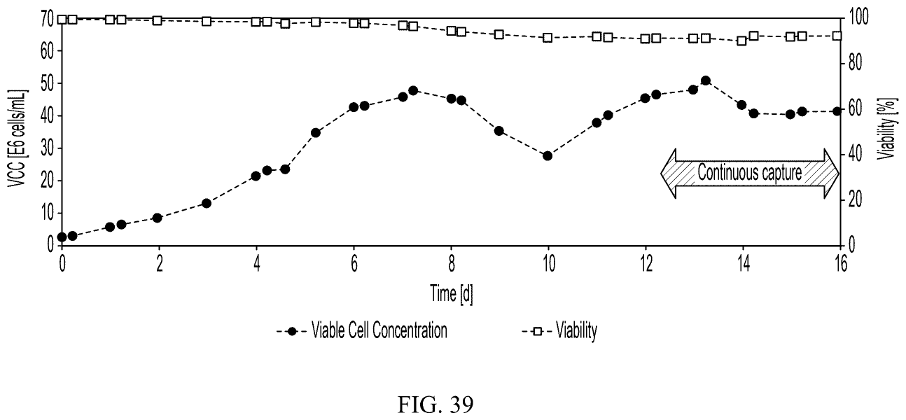

The cell growth and viability of the perfusion cell cultivation is shown in FIG. 39. As a result of the cultivation conditions by the applied control loops, a high viability and similar fast cell growth as previously reported for this cell line was observed during the initial growth phase. An automated cell bleed was started on day 5 just before the target VCC of 45.106 cells/mL was obtained, assisting with a smooth transition to the steady-state like phase of the process (FIG. 39). The target VCC could be well controlled for the remaining perfusion operation with only one noticeable deviation on day 10. Here the actual VCC was about 30% below the target VCC (FIG. 39), which is believed to have resulted from changes in the VCC-capacitance-correlation and thus deviations of on-line and off-line VCC measurements. While the process stabilized quickly after performing a recalibration, this deviation impacted the current bleed flow rate and thereby also the permeate flow rate due to the implemented control strategy described above. However, the continuous product capture from the perfusion permeate was implemented between cultivation days 12 to 16. During this period the average mAb concentration in the permeate stream was approximately 0.7 g/L.

### Adaptive Control Strategies for Dynamic Flow and Loading for Continuous Capture

For a direct continuous capture of the mAb from the perfusion cultivation a continuous affinity SMB chromatography was used. In order to establish a robust process and to be able to respond to fluctuations of the USP an overarching advanced control strategy was developed.

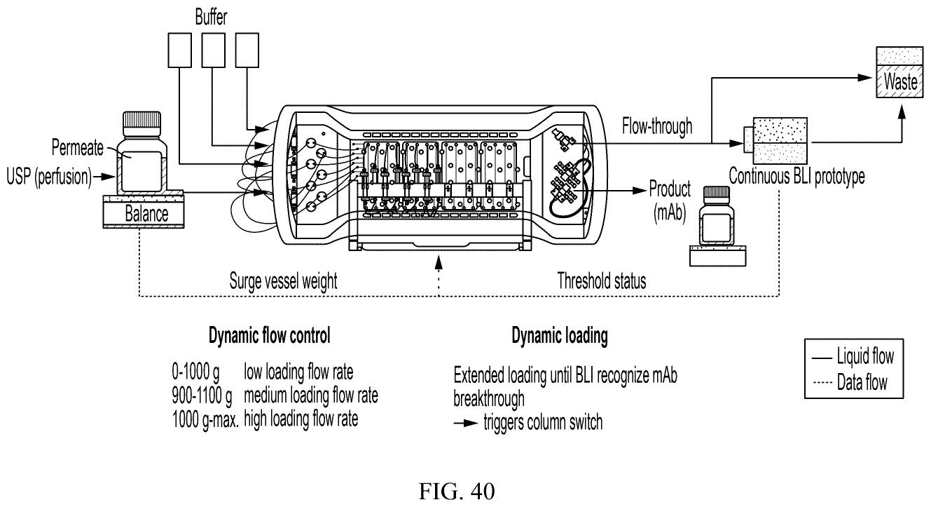

The cell free perfusion permeate was collected in a surge tank placed on a balance. The surge tank was directly connected to the load inlet of the SMB chromatography system. The balance signal was transmitted via an OPC UA interface. For the dynamic flow control, which may compensate for variations in perfusion flow rate from the USP, the loading flow rate of the SMB chromatography system was adjusted to maintain a constant weight of the surge tank of approximately 1 kg. The surge tank weight as well as the current loading flow rate were constantly retrieved by the overarching control strategy. Surge tank weights less than 0.9 kg resulted in a switch from the medium loading flow rate to the low flow rate, while weights above 1.1 kg triggered the high flow rate (FIGS. 38 and 40).

In order to compensate for titer variations within the USP and the different breakthrough times as a result of the different loading flow rates, a dynamic loading function was implemented in the overarching control strategy as well. Based on the initial chromatography recipe the loading as well as the sequential connection to transfer the flow-through of one column to another, was continued until a mAb breakthrough was detected by the continuous BLI prototype in the flow-through of the subsequent column (FIG. 40). After breakthrough detection, the continuous BLI measurement was in stand-by mode for 108 minutes. This corresponds to the minimum loading time of the SMB chromatography based on the highest expected titer throughout the USP to avoid unnecessary measurements.

In order to obtain a synchronization between the SMB chromatography system and the continuous BLI prototype the status of the chromatography system was continuously polled by the overarching control strategy software component. A pause/resume functionality of the overall control script and the associated waiting time until the measurement of the continuous BLI prototype was implemented to ensure a stable process even in the case of unexpected events, such as potential overpressure in the chromatography system (FIG. 38).

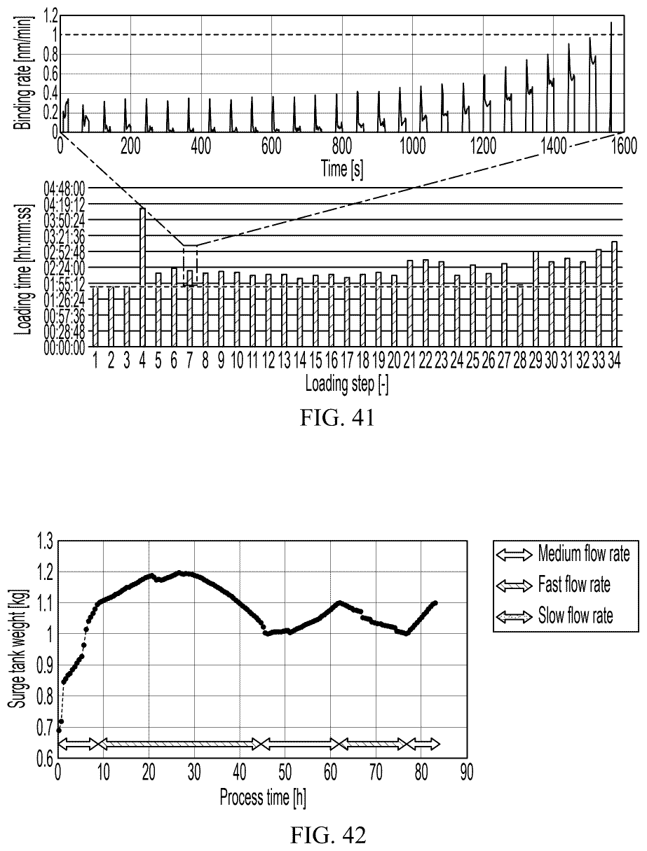

Throughout the continuous capture in total 34 loading steps were performed by the SMB chromatography system within approximately 3 days (FIG. 41). In order to start the process and for an initial loading of the columns, the first 3 loading steps were performed according to the predefined initial recipe without a dynamic control strategy. Accordingly, the loading times for loading steps 1 to 3 were exactly 108 minutes which was the loading time of the initial recipe and corresponds to the highest expected mAb titer of 1 g/L throughout the USP (FIG. 41).

Beginning with the second cycle, corresponding to the fourth loading step, the continuous BLI measurement was automatically started by the overarching control strategy after 108 min loading time. The flow-through of the last (in this setup the second) column in series during the interconnected loading step was monitored for mAb breakthrough. In FIG. 41 (top) the binding rate signal from the continuous BLI prototype is shown exemplary for the seventh loading step. Due to the cyclic measurement principle a binding rate signal was obtained every minute. The binding rate remained at a low baseline level below 0.4 nm up to 800 s measurement time indicating the absence of significant amounts of mAb in the flow-through (FIG. 41). From that time on the value continued to increase representing a gradual breakthrough of mAb from the column. At the 27th cycle of the continuous BLI measurement (starting after 1560 s), the predefined threshold of the binding rate of 1 nm/min was exceeded (FIG. 41), resulting in a stop of the measurement and a column switch of the SMB chromatography by the overarching control strategy (FIG. 38). The mAb concentration in the flow-through at the end of the loading step was determined exemplary for the 7th and 27th continuous BLI measurement to be below 0.02 g/L, indicating no significant product loss due to the dynamic loading.

Throughout the entire process mAb breakthrough was only observed after 108 min (FIG. 41, bottom), which confirmed the calculated shortest expected loading time. The loading time for the fourth loading step was significant longer compared to the other loading steps due to an underloading of the columns within the initial start cycle of the SMB chromatography, which was not controlled by the dynamic loading strategy. Later in the process, longer loading times occur due to a decreased mAb titer in the permeate (Table 1) and because the loading flow rate was partly reduced by the dynamic flow control in this time range (FIG. 42), resulting in a later mAb breakthrough (FIG. 41, bottom).

After the initial filling of the surge tank at the beginning of the process the first cycle, corresponding to the first three loading steps, were performed based on the predefined recipe with the medium loading flow rate (FIG. 42). For the next cycle, starting from 5.4 h process time, the dynamic flow control strategy has taken effect, resulting in a switch to the fast flow rate after the threshold of 1.1 kg was exceeded after approximately 9 h (FIG. 42). However, due to the slightly increased VCC at the beginning of the continuous capture integration (FIG. 39) and the consequent higher permeate flow rate, the level of the surge tank further increased up to 1.2 kg. However, after the VCC had readjusted back to the target value of approximately 45.106 cells/mL, and maintaining the cell specific perfusion rate, the fast loading flow rate was sufficient to reduce the surge tank weight back to the desired range. After approximately 45 h process time the weight dropped below 1 kg resulting in a switch to the medium flow rate and a subsequent increase of the surge tank level (FIG. 42). Throughout the following process time the surge tank weight varied between 1 and 1.1 kg which was within the desired range according to the control strategy (FIG. 42).

TABLE 1
Concentration of the mAb throughout the 3 process days for the
permeate as well as the different chromatography outlet pools.

| mAb concentration [g/L] | Day 1 | Day 2 | Day 3 |
|---|---|---|---|
| Permeate | 0.807 | 0.759 | 0.551 |
| Eluate | 14.480 | 16.385 | 14.364 |
| Flow-through | 0.013 | 0.013 | 0.013 |
| Wash | 0.080 | 0.164 | 0.013 |
| Waste | 0.013 | 0.014 | 0.013 |

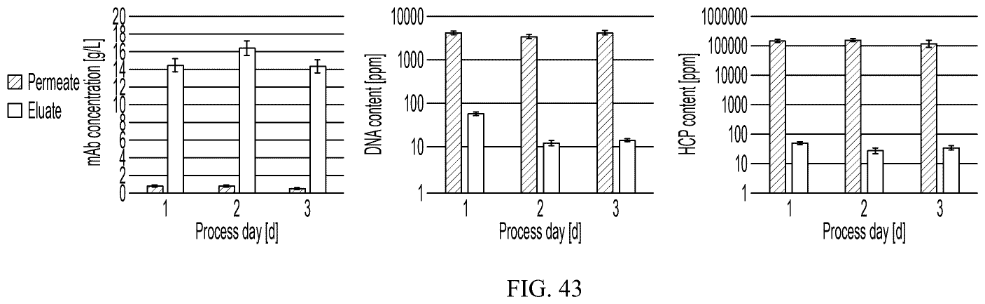

The different outlets of the chromatography system were pooled each for every process day and analyzed together with a permeate sample from the surge tank (Table 1, FIG. 43). Throughout the 3 process days the mAb concentration in the permeate, which was the feed for the continuous SMB chromatography, varied between 0.81 g/L to 0.55 g/L, probably due to small variations of the VCC and product sieving effects during the USP perfusion process. However, a non-decreasing, consistent mAb concentration in the eluate with a mean value of 15.1 g/L was obtained. This demonstrates an optimal resin utilization by the dynamic loading control strategy, despite variations of the product concentration within the USP.

Throughout the entire process only traces of mAb (0.013 g/L, representing the limit of detection of the SEC used for quantification) were detected into the flow-through, again highlighting no significant product loss by the control strategy. While also the pooled waste fractions exhibit no significant mAb concentration, small amounts of the product were detected in the wash fractions, probably due to a too short interconnected column wash step. However, for the complete process a high mAb yield of 96.9% was obtained related to the total mass of mAb recovered in all outlets.

Simultaneously, process related impurities were significantly removed throughout the process. DNA was removed to an average amount of 30.3 ppm, representing a log reduction of 2.1 (FIG. 43). Moreover, a log reduction of 3.6 was obtained for HCP resulting in an average amount of 37.5 ppm in the eluate (FIG. 43).

### Discussion and Conclusion

Due to the implemented USP control strategies for the perfusion feed, cell bleed and perfusion permeate, cultivation conditions with sufficient supply of nutrients were obtained resulting in a robust and well controlled process enabling the maintenance of high cell densities at simultaneous high viabilities.

The resulting variations in the mAb concentration as well as potential alterations or differences of the column binding capacity were addressed by the overarching control strategy, which allowed to carry on the benefits of such an intensified USP to the DSP. Due to the dynamic loading control, column loading was automatically adjusted to obtain good resin utilization without significant product loss. In addition, a robust continuous process was provided by the dynamic flow control which maintained the permeate surge tank within the desired range throughout the complete process. Resulting changes of the loading flow rate, which in turn changed the breakthrough time point during the loading step, were also addressed by the dynamic loading. This further highlights the interaction and mutual benefit of the two applied DSP control strategies.

The advantages become even more obvious when comparing the results of this study with a similar process without adaptive control. An over 100% increase of the mAb concentration in the eluate was obtained by good resin utilization due to the dynamic loading compared to the conservative predefined recipe, while the impurity level remains equal. This also results in improvements in productivity for the subsequent DSP unit operations.

The novel continuous BLI prototype used in this study was able to reliably detect the mAb breakthrough for every loading step. Only very low background signals were obtained, which probably represent small amounts of leached mAb during the loading. Throughout the entire process no adjustment of the threshold or exchange of the biosensor was needed despite over 1000 measurement cycles were performed.

In summary, the novel prototype for continuous BLI exhibits a variety of benefits. SPR technology may also be used to for the advanced control of bioprocessing unit operations, such as mAb capture. In addition, the use of different biosensors with other immobilized ligands at the surface, offers great potential for the monitoring and process control of several biopharmaceuticals such as recombinant proteins, viruses, exosomes and others. Moreover, quality attributes like HCP content, glycosylation patterns or binding kinetics could be investigated at-line using appropriate biosensors and system setups.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. (canceled)

2. A system, comprising:
a first instrument comprising a probe and an optical detector configured to detect a variation of an optical signal over a first period of time; and
a bioprocessing system,
wherein the system is configured to supply a fluid from the bioprocessing system to the first instrument,
wherein the first instrument is configured to determine a rate at which an analyte becomes immobilized on the probe while the fluid contacts and flows over the probe based on the variation of the optical signal over the first period of time,
wherein the optical signal comprises light reflected from an interface internal to the probe and light reflected from the end of the probe, and
wherein the system is configured to send instructions to the bioprocessing system based on the determination of the rate at which the analyte becomes immobilized on the probe.

3. A method, comprising:
contacting a probe with a fluid over a first period of time, wherein the fluid is supplied by a bioprocessing system, wherein the fluid is flowing over the probe, wherein an analyte is present in the fluid at a first concentration, and wherein at least a portion of the analyte becomes immobilized on the probe;
detecting a variation of an optical signal over the first period of time; and
determining a rate at which the analyte becomes immobilized on the probe based on the variation of the optical signal over the first period of time, wherein the optical signal comprises light reflected from an interface internal to the probe and light reflected from the end of the probe; and
based on the determination of the rate at which the analyte becomes immobilized on the probe, sending instructions to the bioprocessing system.

4. A first instrument, comprising:
a probe; and
an optical detector configured to detect a variation of an optical signal over a first period of time,
wherein the first instrument is configured to determine a first concentration of an analyte in a fluid contacting and flowing over the probe based on the variation of the optical signal over the first period of time, and
wherein the optical signal comprises light reflected from an interface internal to the probe and light reflected from the end of the probe.

5. (canceled)

6. The system as in claim 2, wherein the fluid is supplied to the first instrument as output from the bioprocessing system, and wherein the fluid is supplied to the first instrument in an automated manner.

7-8. (canceled)

9. The system as in claim 2, wherein the system comprises a second bioprocessing system, and wherein the second bioprocessing system is configured to supply a second fluid output from the second bioprocessing system to the first instrument.

10-14. (canceled)

15. The system as in claim 2, wherein the bioprocessing system comprises a chromatography system, a filtration system, and/or a bioreactor.

16. The system as in claim 15, wherein the system further comprises a second bioprocessing system, and wherein the second bioprocessing system comprises a bioreactor.

17-34. (canceled)

35. The method as in claim 3, wherein the bioprocessing system comprises a chromatography system, and wherein the method further comprises washing a column from which the fluid is supplied, eluting a column from which the fluid is supplied, and/or regenerating the column from which the fluid is supplied.

36-46. (canceled)

47. The method as in claim 3, wherein the fluid contacted with the probe is a first sample supplied by a source of samples, and further comprising:
closing a first valve to remove the source of samples from fluidic communication with the probe;
opening a second valve to place a source of a regeneration fluid in fluidic communication with the probe; and
contacting the probe with the regeneration fluid,
wherein opening the first valve supplies the sample directly to the probe and/or opening the second valve supplies the regeneration fluid directly to the probe.

48. The system as in claim 2, further comprising:
a plurality of valves positioned upstream of the probe, wherein:
the plurality of valves comprises a first valve positioned between a source of samples and the probe and a second valve positioned between a source of a regeneration fluid and the probe, and
opening the first valve supplies the sample directly to the probe and/or opening the second valve supplies the regeneration fluid directly to the probe.

49. The method as in claim 3, wherein the method is performed in a first instrument, wherein the fluid is a first sample, wherein the probe is a first probe, and further comprising:
in the first instrument, performing the steps of:
contacting the first probe with a first sequence of fluids, wherein the first sequence of fluids comprises the first sample and a regeneration fluid,
contacting a second probe with a second sequence of fluids, wherein the second sequence of fluids comprises a second sample and the regeneration fluid, and
subsequent to contacting the first probe with the regeneration fluid, contacting the first probe with a third sample.

50-77. (canceled)

78. The system as in claim 2, further comprising a valve positioned upstream of the probe, wherein the valve is configured to switch between a source of the fluid, a source of a regeneration fluid, and a source of a neutralization fluid.

79-84. (canceled)

85. The method as in claim 3, wherein the first instrument comprises a valve positioned upstream of the probe, wherein the valve is configured to switch between a source of the fluid, a source of a regeneration fluid, and a source of a neutralization fluid, and wherein the method further comprises switching the valve to remove the source of the regeneration fluid from fluidic communication with the probe and place the source of the neutralization fluid in fluidic communication with the probe.

86-106. (canceled)

107. The system as in claim 78, wherein the first instrument further comprises a source of a dilutant, and wherein the first instrument is configured to mix the dilutant with the plurality of samples upstream of the valve.

108-192. (canceled)

193. The system as in claim 2, wherein the first instrument comprises a plurality of light sources, each associated with a different probe.

194. The system as in claim 2, wherein the first instrument comprises one or more light sources, and wherein two or more probes are associated with a single light source.

195-230. (canceled)

231. The system as in claim 2, wherein the first instrument is interfaced with an additional instrument performing bioprocess, and wherein the first instrument monitors the bioprocess.

232-236. (canceled)

237. The system as in claim 2, wherein the instructions comprise instructions to modify one or more properties of a fluid in the bioprocessing system, to supply the fluid to a different location, to pause, and/or to take no action.

238-241. (canceled)

242. The system as in claim 15, wherein the chromatography system comprises a Multi Column Chromatography (MCC) system.

243. The method as in claim 3, further comprising determining a binding capacity of a column for the analyte.

244. The method as in claim 3, wherein the bioprocessing system comprises a column, and wherein the instructions comprise instructions as to whether to continue to supply fluid upstream of the column to the column.

245. The method as in claim 78, wherein the neutralization fluid is configured to remove any undesirable species deposited on the probe from the regeneration fluid.

246. The method as in claim 3, wherein the immobilization of the analyte on the probe causes an increase in thickness at the end of the probe, thereby causing an increased wavelength shift and an increased binding signal.

247. The method as in claim 3, further comprising determining the first concentration of the analyte, determining an association constant of the analyte, and/or the determining a dissociation constant of the analyte based on the rate at which the analyte becomes immobilized on the probe.

248. The method as in claim 78, wherein the regeneration fluid is configured to cause detachment of at least a portion of any analyte immobilized on the probe.

249. A method, comprising:
contacting a probe with a fluid over a first period of time, wherein the fluid is supplied by a first bioprocessing system, wherein the fluid is flowing over the probe, wherein an analyte is present in the fluid at a first concentration, and wherein at least a portion of the analyte becomes immobilized on the probe;
detecting a variation of an optical signal over the first period of time; and
determining the first concentration based on the variation of the optical signal over the first period of time wherein the optical signal comprises light reflected from an interface internal to the probe and light reflected from the end of the probe; and
based on the determination of the first concentration, sending instructions to a second bioprocessing system.
